(19) United States
(12) Patent Application Publication
(10) Pub. No.: US 2006/0009425 A1
(43) Pub. Date: Jan. 12, 2006

(54) Oral formulations of paricalcitol

(76) Inventors: Leticia Delgado-Herrera, Lake Forest, IL (US); Steve Chamberlin, Waukegan, IL (US); Dennis Stephens, Mt. Prospect, IL (US); Joel Melnick, Wilmette, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/137,611

(22) Filed: May 25, 2005

Related U.S. Application Data
(60) Provisional application No. 60/575,620, filed on May 28, 2004.
(60) Provisional application No. 60/621,700, filed on Oct. 25, 2004.

(51) Int. Cl. A61K 31/59

(52) U.S. Cl. 514/167

(57) ABSTRACT

The present invention relates to oral formulations comprising paricalcitol that are available in a variety of different dosage forms that are bioequivalent to one another.

### CROSS-REFERENCE TO RELATED APPLICATIONS

This application is related to the following U.S. Patent applications: Application No. 60/575,620, filed May 28, 2004 and Application No. 60/621,700, filed Oct. 25, 2004.

### FIELD OF THE INVENTION

This invention relates to pharmaceutical formulations. More particularly, the present invention relates to oral formulations comprising paricalcitol and a non-polar solvent. The oral formulations of the present invention are available in a variety of different dosage strengths that are bioequivalent, and provide equivalent clinical utility as an intravenous paricalcitol formulation. Additionally, the oral formulations of the present invention can be used to reduce the level of parathyroid hormone in patients suffering from chronic kidney disease with no significant difference in the incidences of hypercalcemia and hyperphosphatemia when compared to placebo. In this regard, the oral formulations of the present invention have been found to be equally effective and safe in reducing the levels of parathyroid hormone in chronic kidney disease patients regardless of whether said formulations are administered daily (abbreviated as “QD”) or three times a week (abbreviated as “TIW”) to chronic kidney disease patients in need of such treatment.

### BACKGROUND OF THE INVENTION

Vitamin D (also known as the vitamin D receptor activator (abbreviated as “VDR activator”)) is essential for life in higher animals as it is an important regulator of calcium and phosphorus. More specifically, vitamin D is required for the proper development and maintenance of bone. Typically, vitamin D acts on the intestine, bone, kidney and parathyroid glands to control serum calcium levels. The major circulating form of vitamin D is 25(OH)D₃, which is hydroxylated in the kidneys to the metabolically active form 1,25(OH)₂D₃. It is this metabolically active form of vitamin D that is necessary for the excretion of phosphate in animals.

Another important regulator of calcium and phosphorus in animals is the parathyroid hormone (“PTH”). PTH is secreted from the cells of the parathyroid gland and targets cells in the bone and kidney. PTH is released in response to low extracellular concentrations of free calcium. When serum calcium concentrations fall below the normal range, there is a steep increase in the secretion of PTH. Nonetheless, low levels of PTH are secreted when blood calcium levels are high.

As alluded to above, the main function of vitamin D is to increase calcium absorption from the intestine and promote normal bone formation and mineralization. This function is mediated by a receptor that is a transcription factor, and which is instrumental in turning on a number of genes that express the biologic activity of vitamin D hormone. The hydroxylation of 25(OH)D₃ in the kidneys is strongly stimulated by PTH and, independently of PTH, by hypophosphatemia.

Patients suffering from chronic kidney disease (abbreviated as “CKD”) slowly lose kidney function over a period of time. CKD is currently defined as kidney damage, confirmed by a kidney biopsy or characterized by markers of kidney damage, or a glomerular filtration rate (abbreviated as “GFR”)<60 mL/min/1.73 m² for three months. Kidney damage is defined as pathological abnormalities or makers of damage, including abnormalities in blood or urine tests or imaging studies. Markers of kidney damage include proteinuria, abnormalities on the urine dipstick or sediment examination, or abnormalities on imaging studies of the kidneys. GFR can be estimated from prediction equations based on serum creatinine and other variables, including age, sex, race, and body size.

Among individuals with CKD, the stage of the disease (see below in Table A which is taken from the National Kidney Foundation Kidney Disease Quality Initiative (K/DOQI), “Clinical Practice Guidelines for Bone Metabolism in Chronic Kidney Disease,” American Journal of Kidney Diseases, 42(4), Supp. 3, S1-S201 (October 2003)) is based on the level of GFR, irrespective of the cause of kidney disease.

TABLE A

| Stage | | GFR (mL/min/1.73 m²) |
|---|---|---|
| 1 | Kidney damage with normal or | ≧90 |
| | increased GFR | |
| 2 | Kidney damage with mild decrease | 60-89 |
| | in GFR | |
| 3 | Moderate decrease in GFR | 30-59 |
| 4 | Severe decrease in GFR | 15-29 |
| 5 | Kidney Failure (End-Stage Renal | <15 (or dialysis) |
| | Disease) | |

CKD patients become unable to make metabolically active vitamin D and become inefficient at excreting phosphate. As a result, their levels of metabolically active vitamin D drop, causing a drop in circulating blood calcium levels and an increase in circulating blood phosphate levels. In an attempt to compensate for the decrease in circulating blood calcium levels, the parathyroid gland secretes PTH to normalize the calcium and phosphate levels. Eventually, the secretion of PTH becomes excessive. This excessive secretion of PTH is referred to as secondary hyperparathyroidism (abbreviated as “2° HPT”). As will be discussed in more detail below, patients with PTH levels higher than those in the recommended range are at greater risk for bone disorders. The National Kidney Foundation Kidney Disease Outcomes Quality Initiative (K/DOQI) guideline (“Clinical Practice Guidelines for Bone Metabolism in Chronic Kidney Disease,” American Journal of Kidney Diseases, 42(4), Supp. 3, S1-S201 (October 2003)) recommends treatment when PTH levels are greater than 70 pg/mL to prevent or ameliorate bone disease.

Despite the increase in the levels of circulating PTH, the kidneys typically remain unable to produce any metabolically active vitamin D and more PTH is secreted. However, despite the continued increase in the levels of circulating PTH, the kidneys do not respond. After a while, the levels of circulating phosphate become so elevated that the phosphate combines with the circulating calcium to form calcium phosphate crystals in the soft tissue of the patient. The removal of this calcium from circulation causes the bones to release all available calcium. This release of calcium causes the bones to become soft and bendable. Eventually, by the time that these patients reach the end stage of the disease (known as “end stage renal disease” or “ESRD”), their kidneys function less than 10% of the baseline and are no longer remain able to function at the level necessary for day-to-day life. At this point, these patients need to undergo dialysis treatment or receive a kidney transplant.

As alluded to above, CKD is associated with a variety of bone disorders. The major disorders of bone can be classified into those associated with PTH levels (osteitis fibrosa cystica) and those with low or normal PTH levels (adynamic bone disease) (“Clinical Practice Guidelines for Bone Metabolism in Chronic Kidney Disease,” American Journal of Kidney Diseases, 42(4), Supp. 3, S1-S201 (October 2003)). The hallmark lesion of chronic kidney disease is osteitis fibrosa, due to 2° HPT. Id. Nonetheless, irrespective of the cause, bone disease can lead to pain and an increased incidence of fractures. Id. Abnormal calcium-phosphorus metabolism and hyperparathyroidism can also lead to calcification of blood vessels and potentially an increased risk of cardiovascular events. Id.

Unfortunately, the stage of chronic kidney disease at which bone disease begins to develop has not been well documented. Id. Moreover, a consensus has not been developed regarding the best screening measures for detecting early abnormalities of calcium-phosphorus metabolism and bone disease. Id. Bone disease associated with chronic kidney disease is composed of a number of abnormalities of bone mineralization. Id. The major disorders can be classified into those associated with high bone turnover and high PTH levels (including osteitis fibrosa, the hallmark lesion of 2° HPT, and mixed lesions) and low bone turnover and low or normal PTH levels (osteomalacia and adynamic bone disease). Id. Osteomalacia may be related to vitamin D deficiency, excess aluminum, or metabolic acidosis; whereas adynamic bone disease may be related to over-suppression of PTH with calcitriol. Id.

The pathophysiology of bone disease due to 2° HPT is related to abnormal mineral metabolism: (1) decreased kidney function leads to reduced phosphorus excretion and consequent phosphorus retention; (2) elevated serum phosphorus can directly suppress calcitriol (1,25-dihydroxyvitamin D₃) production; (3) reduced kidney mass leads to decreased calcitriol production; (4) decreased calcitriol production with consequent reduced calcium absorption from the gastrointestinal tract contributes to hypocalcemia, as does abnormal calcium-phosphorus balance leading to an elevated calcium-phosphorus product. Id. Hypocalcemia, reduced calcitriol synthesis, and elevated serum phosphorus levels stimulate the production of PTH and the proliferation of parathyroid cells, resulting in 2° HPT. Id. High PTH levels stimulate osteoblasts and result in high bone turnover. The hallmark lesion of 2° HPT is osteitis fibrosa cystica. Id. High bone turnover leads to irregularly woven abnormal osteoid, fibrosis, and cyst formation, which result in decreased cortical bone and bone strength and an increased risk of fracture. Id. Low turnover bone disease has two subgroups, osteomalacia and adynamic bone disease. Both lesions are characterized by a decrease in bone turnover or remodeling, with a reduced number of osteoclasts and osteoblasts, and decreased osteoblastic activity. Id. In osteomalacia there is an accumulation of unmineralized bone matrix, or increased osteoid volume, which may be caused by vitamin D deficiency or excess aluminum. Id. Adynamic bone disease is characterized by reduced bone volume and mineralization and may be due to excess aluminum or oversuppression of PTH production with calcitriol. Id. Bone biopsy following double-tetracycline labeling is the gold standard for the diagnosis of bone disease in chronic kidney disease and is the only means of definitively differentiating them. Five bone lesions associated with chronic kidney disease have been classified based on bone formation rate, osteoid area, and fibrosis on bone biopsy of patients with kidney failure (See Table B, below).

TABLE B

| | Bone Formation | | |
|---|---|---|---|
| | Rate (μm²/mm² | Osteoid | Fibrosis |
| Lesions | Tissue area/day) | Area (%) | (%) |
| Aplastic | <108 | <15% | <0.5% |
| (adynamic): | | | |
| Osteomalacia: | <108 | <15% | <0.5% |
| Mild: | <108 | <15% | <0.5% |
| Osteitis Fibrosa: | <108 | <15% | <0.5% |
| Mixed: | <108 | <15% | <0.5% |

Several bone markers have been identified as having been correlated with bone disorders in patients suffering from CKD. Two extensively studies markers include PTH and bone alkaline phosphatase (“b-Alk Phos”)). PTH secretion is directly correlated with bone turnover, but PTH levels are not reliably correlated with bone turnover among dialysis patients, especially in the middle ranges. PTH levels <65 pg/mL were found to be predictive of normal bone or low turnover lesions, and PTH levels >450 pg/mL were predictive of high turnover lesions, but levels in between did not have good predictive value. Overall bone turnover could not be predicted in 30% of HD and 50% of PD patients. Id. In another study, low turnover lesions were noted in the majority of patients with PTH levels <100 pg/mL and high turnover lesions in the majority of patients with PTH levels >200 to 300 pg/mL. Id. High b-Alk Phos levels have been associated with high bone turnover and low levels with adynamic bone disease in dialysis patients. Id. In one study, the combination of high b-Alk Phos levels with high PTH levels increased the sensitivity of diagnosis of high turnover lesions; conversely, low levels of both of these markers result in increased sensitivity for diagnosis of low turnover lesions. Id. However, specific cut-off levels for b-Alk Phos have varied in the few studies examining the relationship to bone histology. Id.

Other markers of bone disease that have been investigated include osteocalcin, β2 microglobulin, procollagen type I carboxy-terminal propeptides (PICP), and type I collagen cross linked telopeptides (ICTP), urinary pyridinoline, deoxypyridinoline and others. PICP has been correlated with bone formation, and ICTP and osteocalcin been correlated with bone resorption.

As mentioned above, patients suffering from CKD typically also suffer from 2° HPT. Vitamin D and vitamin D analogs, such as doxercalciferol and alfacalcidol, and Vitamin D receptor activators such as calcitriol, maxacalcitol and Falecalcitriol and selective vitamin D receptor activators, such as paricalcitol, have been used to suppress excess PTH levels in patients suffering from CKD. These vitamin D, vitamin D analogs, Vitamin D receptor activators and selective Vitamin D receptor activators are traditionally administered to these patients intravenously, although a few oral formulations are commercially available. Unfortunately, one of the side effects associated with the administration of Vitamin D, Vitamin D analogs and some selective Vitamin D receptor activators, such as maxacalcitol to CKD patients is hypercalcemia and hyperphosphatemia. Interestingly, studies have shown that the incidences of hypercalcemia and hyperphosphatemia are greatly reduced when a vitamin D or vitamin D analog is administered to CKD patients intravenously instead of in an oral formulation. For example, Dennis L. Andress in a paper entitled “Intravenous Versus Oral Vitamin D Therapy in Dialysis Patients: What is the Question,” American Journal of Kidney Diseases, 38(5), Supp. 5: S41-S44 (November 2001), reports that the use of intermittent (pulse) oral calcitriol (tradename Rocaltrol®) therapy in dialysis patients has been shown to have a greater incidence of hypercalcemia and hyperphosphatemia in controlled studies that compared oral with IV calcitriol therapy. In fact, Andress goes on to recommend that “[Because oral calcitriol is less effective than parenteral calcitriol in long-term studies and its use is completely dependent on full patient compliance, we recommend that oral calcitriol should not be used for intermittent vitamin D therapy” (page S43, emphasis added).

High incidences of hypercalcemia and hyperphosphatemia in dialysis patients have also been reported with the use of oral doxercalciferol (tradename Hectorol®), which is a synthetic vitamin D analog when compared with IV doxercalciferol therapy.

There is a need in the art for an oral formulation of a vitamin D analog that can be used to treat 2° HPT and which does not cause high incidences of hypercalcemia and hyperphosphatemia. Additionally, there is also a need for an oral formulation of a vitamin D analog that exhibits a pharmacokinetic and therapeutic (i.e., safety and efficacy) profile that is comparable to the intravenous administration of said vitamin D analog.

Moreover, given the nature of the continuum of CKD progression, a clinician may prescribe very low doses (i.e., 1 mcg three times a week) or high doses (e.g., 50 mcg three times per week) to a patient in need of treatment. In order to increase flexibility of dosing as well as ease of patient use, there is a need in the art to have available a variety of different capsule strengths of a vitamin D analog. Such a variety of capsule strengths would minimize the number of pills that a patient would have to ingest to obtain the desired dose. This increases the convenience to the patient and facilitates improved patient compliance.

Furthermore, a clinician must be assured that if he/she prescribes using different capsule strengths that the doses are bioequivalent. What this means is that a clinician must be assured that if a dose of 8 mcg is prescribed, for example, that the bioavailable fraction of drug that the patient receives and the associated kinetic profile of the dose is the same whether the patient takes one 8 mcg capsule or eight 1 mcg capsules. Therefore, there also a need in the art for oral formulations of varying dosage strengths of a vitamin D analog that are bioequivalent to one another.

### SUMMARY OF THE INVENTION

In one embodiment, the present invention relates to any member of a family of oral formulations that comprise a therapeutically effective amount of paricalcitol dissolved in an amount of a non-polar solvent. Each of said family members comprises a ratio of non-polar solvent to paricalcitol. This ratio of non-polar solvent to paricalcitol does not vary by more than a factor of about 4, preferably not more than by a factor of about 3.5, more preferably not more than by a factor of about 3.0, and most preferably, not more than by a factor of about 2.0, from a ratio of non-polar solvent to paricalcitol in a selected reference oral formulation that is also a member of the family. Additionally, each family member, when dosed at the same total weight of paricalcitol, is bioequivalent to the selected reference oral formulation and to one another, and provides equivalent clinical utility to an intravenous formulation,

In another embodiment, the present invention relates to any member of a family of oral formulations that comprises: (a) about 0.25 mcg of paricalcitol dissolved in an amount of a non-polar solvent; (b) about 0.50 mcg of paricalcitol dissolved in an amount of a non-polar solvent; (c) about 0.75 mcg of paricalcitol dissolved in an amount of a non-polar solvent; (d) about 1.0 mcg of paricalcitol dissolved in an amount of a non-polar solvent; (e) about 2.0 mcg of paricalcitol dissolved in an amount of a non-polar solvent; (f) about 3.0 mcg of paricalcitol dissolved in an amount of a non-polar solvent; (g) about 4.0 mcg of paricalcitol dissolved in an amount of a non-polar solvent; (h) about 8.0 mcg of paricalcitol dissolved in an amount of a non-polar solvent; (i) about 16.0 mcg of paricalcitol dissolved in an amount of a non-polar solvent; or (j) about 32.0 mcg of paricalcitol dissolved in an amount of a non-polar solvent. Each of the above family members comprises a ratio of non-polar solvent to paricalcitol. This ratio of non-polar solvent to paricalcitol does not vary by more than a factor of about 4, preferably not more than a factor of about 3.5, more preferably not more than by a factor of about 3.0, and most preferably, not more than by a factor of about 2.0, from a ratio of non-polar solvent to paricalcitol in a selected reference oral formulation that is also a member of the family. Additionally, each family member, when dosed at the same total weight of paricalcitol, is bioequivalent to the selected reference formulation and to one another, and provide equivalent clinical utility to an intravenous formulation.

In yet another embodiment, the present invention relates to any member of a family of oral formulations that comprises: (a) about 0.25 mcg of paricalcitol dissolved in an amount of a non-polar solvent; (b) about 0.50 mcg of paricalcitol dissolved in an amount of a non-polar solvent; (c) about 0.75 mcg of paricalcitol dissolved in an amount of a non-polar solvent; (d) about 1.0 mcg of paricalcitol dissolved in an amount of a non-polar solvent; (e) about 2.0 mcg of paricalcitol dissolved in an amount of a non-polar solvent; (f) about 3.0 mcg of paricalcitol dissolved in an amount of a non-polar solvent; (g) about 4.0 mcg of paricalcitol dissolved in an amount of a non-polar solvent; (h) about 8.0 mcg of paricalcitol dissolved in an amount of a non-polar solvent; (i) about 16.0 mcg of paricalcitol dissolved in an amount of a non-polar solvent; or (O) about 32.0 mcg of paricalcitol dissolved an amount of a non-polar solvent, provided that the family members containing 2.0 mcg and 4.0 mcg of paricalcitol do not each contain 140.56 mg of a non-polar solvent. Each of the above family members comprises a ratio of non-polar solvent to paricalcitol. This ratio of non-polar solvent to paricalcitol does not vary by more than a factor of about 4, preferably not more than by a factor of 3.5, more preferably not more than by a factor of about 3.0, and most preferably, not more than by a factor of about 2.0, from a ratio of non-polar solvent to paricalcitol in a selected reference oral formulation that is also a member of the family. Additionally, each family member, when dosed at the same total weight of paricalcitol, is bioequivalent to the selected reference formulation and to one another, and provide equivalent clinical utility to an intravenous formulation.

In yet a further embodiment, the present invention relates to a family of oral formulations that are made pursuant to a method. One step in said method involves providing a first oral formulation comprising paricalcitol and a non-polar solvent. This first oral formulation contains a first ratio of non-polar solvent to paricalcitol. A second step in said method involves preparing any number of additional oral formulations comprising paricalcitol and a non-polar solvent. Each of these additional oral formulations comprises a second ratio of non-polar solvent to paricalcitol. This second ratio of non-polar solvent to paricalcitol in each additional oral formulation does not vary by more than a factor of about 4, preferably not more than by a factor of 3.5, more preferably not more than by a factor of about 3.0, and most preferably, not more than by a factor of about 2.0, from the first ratio. Additionally, each of the first and additional oral formulations of said family, when dosed at the same total weight of paricalcitol, prepared pursuant to the steps of this method are bioequivalent to each other.

In yet still a further embodiment, the present invention relates to a method of making a family of oral formulations that are bioequivalent to one another. One step in said method involves providing a first oral formulation comprising paricalcitol and a non-polar solvent. This first oral formulation contains a first ratio of non-polar solvent to paricalcitol. A second step in said method involves preparing any number of additional oral formulations comprising paricalcitol and a non-polar solvent. Each of these additional oral formulations comprises a second ratio of non-polar solvent to paricalcitol. This second ratio of non-polar solvent to paricalcitol in each additional oral formulation does not vary by more than a factor of about 4, preferably not more than a factor of about 3.5, more preferably not more than by a factor of about 3.0, and most preferably, not more than by a factor of about 2.0, from the first ratio. Additionally, each of the first and additional oral formulations of said family, when dosed at the same total weight of paricalcitol, prepared pursuant to the steps of this method are bioequivalent to each other.

In yet still a further embodiment, the present invention relates to another method for making a family of oral formulations that are bioequivalent. One step of the method involves providing a first oral formulation comprising paricalcitol and a non-polar solvent. This first oral formulation contains a first ratio of non-polar solvent to paricalcitol. A second step in said method involves preparing a second oral formulation comprising paricalcitol and a non-polar solvent. This second oral formulation contains a second ratio of non-polar solvent to paricalcitol. Additionally, the second ratio of non-polar solvent to paricalcitol does not vary by more than a factor of about 4, preferably not more than a factor of about 3.5, more preferably not more than by a factor of about 3.0, and most preferably, not more than by a factor of about 2.0, from the first ratio. Additionally, each of the first and second oral formulations of said family, when dosed at the same total weight of paricalcitol and prepared pursuant to the steps of this method, is bioequivalent to each other.

Another step in said method involves preparing a third oral formulation comprising paricalcitol and a non-polar solvent. This third oral formulation contains a third ratio of non-polar solvent to paricalcitol. Additionally, the third ratio of non-polar solvent to paricalcitol in the third oral formulation does not vary by more than a factor of about 4, preferably not more than a factor of 3.5, more preferably not more than by a factor of about 3.0, and most preferably, not more than by a factor of about 2.0, from the first ratio. Additionally, each of the first, second and third oral formulations of said family prepared pursuant to the steps of this method are bioequivalent to each other, when dosed at the same total weight of paricalcitol.

In yet still a further embodiment, the present invention relates to a method of suppressing parathyroid hormone in patients suffering from chronic kidney disease and in need of treatment. This method involves the step of orally administering any member of the family of oral formulations described herein to a patient. The patient receiving said oral formulation can be a mammal, such as a human being, that is suffering from chronic kidney disease, such as pre-end stage or end-stage renal disease. Pursuant to this method, any member of the family of oral formulations described herein can be administered to a patient either daily or three times a week, depending upon the patient.

### DETAILED DESCRIPTION OF THE INVENTION

As mentioned previously, the present invention relates to oral formulations of paricalcitol and to members of a family of oral formulations of paricalcitol. As used herein, the term “paricalcitol” refers to a synthetic vitamin D analog or selective Vitamin D receptor activator having the following structure:

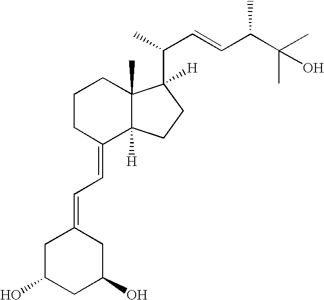

Paricalcitol is also known as 19-nor-1α,3β,25-trihydroxy-9,10-secoergosta-5(Z); 7(E),22(E)-triene, 1α, 25 dihyroxy 19 nor ergocalciferol, 19-nor-1α, 25-dihydroxyvitamin D₂ and 1,α, 25-dihydroxyl-19 nor-vitamin D₂.

Paricalcitol injection is available commercially as Zemplar® from Abbott Laboratories, Abbott Park, Ill. Paricalcitol is a third generation Vitamin D analog commercially available having a structural modification on the side chain and A ring. Methods for the synthesis of paricalcitol are described in U.S. Pat. Nos. 5,246,925, 5,237,110, 5,342,975 and 5,587,497, each herein incorporated by reference.

U.S. Pat. No. 6,136,799, incorporated herein by reference, describes a sterilized, self-preserved, aqueous pharmaceutical composition for parenteral administration. This composition consists essentially of a therapeutically effective amount of a vitamin D compound, such as paricalcitol, about 50% (v/v) of an organic solvent and about 50% (v/v) water. The organic solvent is a low molecular weight alcohol in the range of about 15% to 30% (v/v) and glycol derivatives in the range of about 20% to about 35% (v/v).

A paricalcitol (Zemplar®) injection such as that described in U.S. Pat. No. 6,136,799 has been approved by the FDA and is marketed for the prevention and treatment of 2° HPT associated with chronic renal failure (CKD Stage 5 or end-stage renal disease (ESRD), GFR <15 mL/min). This intravenous formulation contains 2-10 micrograms/milliliter of paricalcitol, 30% (v/v) propylene glycol, 20% (v/v) ethanol and approximately 50% (v/v) water. Well-controlled studies indicate that paricalcitol injection suppresses elevated levels of PTH with minimal effect on serum calcium and phosphorus levels. Since its approval by the FDA in April of 1998, it is estimated that approximately 200,000 patients have received at least 1 dose of paricalcitol injection. Clinically, the safety and efficacy of paricalcitol injection are well established.

I. Definitions

As used herein, the term “AUC” refers to the area under the plasma concentration-time curve and is calculated by the trapezoidal rule. The term “AUC₀₋ₜ” means the area under the plasma concentration curve from time 0 to the last measurable concentration in units of ng·h/mL as determined using the trapezoidal rule. The term “AUC_{0-∞}” means the area under the plasma concentration curve from time 0 to infinite time. AUC(_{0-∞}) is calculated as AUC(₀₋ₜ)+LMT/(−β), where “LMT” is the last measurable plasma concentration and β is the terminal phase elimination rate constant. The term AUC_{0-∞} is also referred to as overall exposure.

As used herein, one formulation (a first formulation) is considered to be “bioequivalent” to another formulation (a second formulation) if there is no significant difference in the rate (Cₘₐₓ) at and extent (AUC₀₋ₜ and AUC_{0-inf}) to which the active ingredient or active moiety in these formulations becomes available at the site of drug action when administered at the same molar dose under similar conditions in an appropriately designed study. This definition is based on “bioequivalence” as defined by the U.S. Food and Drug Administration (Code of Federal Regulations (21 C.F.R. 320.1), incorporated by reference.

As used herein, the term “Cₘₐₓ” refers to the maximum observed plasma concentration.

As used herein, the phrase “equivalent clinical utility” or “clinically equivalent utility” refers to two formulations having similar efficacy and safety. For example, 95% confidence intervals were calculated for the difference in proportions in clinically meaningful efficacy (30% reductions in PTH) and clinically meaningful safety (hypercalcemia) between patients receiving an intravenous formulation and those receiving oral paricalcitol formulations according to the invention would capture zero. A 95% confidence interval for efficacy defined by at least two consecutive 30% reductions in iPTH is given by −26.6% to 5.7% and a 95% confidence interval for safety defining hypercalcemia as two consecutive calcium values greater than 11.0 mg/dL is given by −20.7% to 5.6%, these confidence intervals capture zero which suggests difference between treatment modalities and in addition suggests the true difference between groups in efficacy is less than 27% and less than 21% in regards to safety.

As used herein, the terms “end stage chronic kidney disease” or “end stage renal disease” (ESRD) refer to chronic kidney disease (CKD) stage 5, GFR <15 mL/min.

As used herein, the term “fill” refers to a drug substance (i.e., paricalcitol), non-polar solvent, and other excipients, antioxidants, low molecular weight alcohol, etc., that do not comprise a capsule shell.

As used herein, the term “hypercalcemia” refers to a condition characterized by high levels of calcium in the blood. According to the most current National Kidney Foundation Kidney Disease Quality Initiative (K/DOQI), “Clinical Practice Guidelines for Bone Metabolism in Chronic Kidney Disease,” American Journal of Kidney Diseases, 42(4), Supp. 3, S1-S201 (October 2003), herein incorporated by reference, hypercalcemia is diagnosed if blood serum calcium levels are above 10.2 milligrams per deciliter of blood.

As used herein, the term “hyperphosphatemia” refers to a condition characterized by high levels of phosphate in the blood. According to the most current National Kidney Foundation Kidney Disease Quality Initiative (K/DOQI), “Clinical Practice Guidelines for Bone Metabolism in Chronic Kidney Disease,” American Journal of Kidney Diseases, 42(4), Supp. 3, S1-S201 (October 2003), herein incorporated by reference, hyperphosphatemia is diagnosed if blood phosphate levels are above 5.5 milligrams per deciliter of blood.

As used herein, the term “low molecular weight alcohol” refers to an aliphatic alcohol of from 1 to 5 carbons, i.e., ethanol, propanol, butanol, etc. Ethanol is listed on the United States Food and Drug Administration's (FDA) list of compounds, which are generally recognized as safe (GRAS), and is intended for administration to humans.

As used herein, the term “non-polar solvent” refers to solvents selected from the group consisting of: short chain aliphatic or aromatic hydrocarbons, alkyl-substituted solvents, medium chain triglycerides or mixtures thereof. The non-polar solvent selected for use in the present invention does not react detrimentally with or cause degradation of the paricalcitol. The hydrocarbons of said non-polar solvents contain between 2 to 14 carbon atoms per carbon chain and may contain multiple carbon chains. Preferably, the hydrocarbons are medium chain triglycerides containing between 6 and 12 carbon atoms per carbon chain. Examples of non-polar solvents that can be used in the present invention, include, but are not limited to, caprylic/capric triglyceride (i.e., NEOBEE® M-5, Stepan Company, Northfield, Ill.), canola oil, corn oil, cottonseed oil, ethyl oleate, isopropyl myristate, isopropyl palmitate, light mineral oil, mineral oil, peanut oil or soybean oil.

As used herein, the terms “pre-end stage chronic kidney disease” or “pre-end stage renal disease” (Pre-ESRD) refer to chronic kidney disease (CKD) stages 1-4.

The term “proportionally similar” is defined in the FDA Guidance for Industry document entitled “Bioavailability and Bioequivalence Studies for Orally Administered Drug Products—General Considerations” (March 2003) as follows:
1. All active and inactive ingredients are in exactly the same proportion between different strengths (e.g., a tablet of 50-mg strength has all the inactive ingredients, exactly half that of a tablet of 100-mg strength, and twice that of a tablet of 25-mg strength); or
2. Active and inactive ingredients are not in exactly the same proportion between different strengths as stated above, but the ratios of inactive ingredients to total weight of the dosage form are within the limits defined by the SUPAC-IR and SUPAC-MR guidances up to and including Level II; or
3. For high potency drug substances, where the amount of the active drug substance in the dosage form is relatively low, the total weight of the dosage form remains nearly the same for all strengths (within ±10% of the total weight of the strength on which a biostudy was performed), the same inactive ingredients are used for all strengths, and the change in any strength is obtained by altering the amount of the active ingredients and one or more of the inactive ingredients. The changes in the inactive ingredients are within the limits defined by the SUPAC-IR and SUPAC-MR guidances up to and including Level II.

As used herein, the term “statistically significant,” when used in connection with a statistical test, refers to when the resulting p-value is less than or equal to 0.05, unless otherwise noted.

As used herein, the term “Tₘₐₓ” refers to the time to maximum observed plasma concentration (i.e., the time at which Cₘₐₓ occurred).

As used herein “T_{1/2}” means the terminal phase elimination half-life, in units of hours, determined by simple linear regression of natural log (ln) concentration versus time data points in the “terminal phase” of the concentration time curve. T_{1/2} is calculated as ln(2)/(β). β is the terminal phase elimination rate constant.

The term “therapeutic equivalence” or “therapeutically equivalent” is defined in the FDA Guidance for Industry document entitled “Bioavailability and Bioequivalence Studies for Orally Administered Drug Products—General Considerations” (March 2003) as follows: (1) Approved as both safe and effective; (2) Pharmaceutical equivalents, containing identical amounts of the same active ingredient in the same dosage form and route of administration, and meet compendial standards of strength, quality, purity, and identity; (3) Bioequivalent; (a) do not present a known or potential problem, and meet an acceptable in vitro standard, or (b) if they do present a potential problem, shown to meet an appropriate bioequivalence standard; (4) Adequately labeled; and (5) Manufactured in compliance with the FDA's Good Manufacturing Practices regulations.

All weights referred to are ±10%.

II. Oral Formulations of Paricalcitol and Dosage Forms

As mentioned briefly above, the oral formulations of the present invention comprise an amount of paricalcitol that provides equal equivalent clinical utility as an intravenous paricalcitol formulation to treat a subject in need of treatment, such as, but not limited to, a patient suffering pre-end stage or end-stage renal disease and an amount of a non-polar solvent. In the formulation, the non-polar solvent functions as an excipient. As will be discussed in more detail below, the oral formulations of the present invention can include other ingredients, including additional excipients which can be varied in a manner to make the formulation amenable to manufacture, such as, but not limited to, antioxidants and at least one low molecular weight alcohol.

The oral formulations of the present invention can be prepared in a variety of dosage forms, including tablets, hard capsules and gelatin capsules, among others, and different dosage strengths (e.g., where the concentration of paricalcitol in said formulations is about 0.25 mcg, about 0.50 mcg, about 1.0 mcg, about 2.0 mcg, about 4.0 mcg, about 8.0 mcg, about 16.0 mcg, about 32.0 mcg, etc.) that are bioequivalent, when dosed at the same total weight of paricalcitol, to each other, despite not being compositionally proportional (i.e., not being proportionally similar).

It is well known in the art for solid dosage forms to vary all active and inactive ingredients proportionately (i.e., to make them proportionally similar) in order to prepare bioequivalent products having different dose strengths. Therefore, the discovery of bioequivalent oral formulations of paricalcitol that are not proportionally similar was surprising. Specifically, as shown in Example 1, the inventors of the present invention discovered that when paricalcitol is dissolved in a non-polar solvent, that the ratio of non-polar solvent to paricalcitol is critical in preparing varying dosage strengths that are bioequivalent to one another. In fact, the inventors determined that, once a desired ratio of non-polar solvent to paricalcitol has been determined or designed for a specific dosage strength of paricalcitol of interest (which is referred to as the “selected reference formulation”), other oral formulations having different dosage strengths can be prepared, provided that the non-polar solvent to drug ratio in each of said formulations does not vary by more than a factor of about 4, preferably not more than about 3.5, more preferably not more than a factor of about three (3.0), and most preferably, not more than a factor of two (2.0), to the ratio of non-polar solvent to paricalcitol of the selected reference formulation. Any oral formulation of differing dosage strength containing a non-polar solvent to drug ratio that does not vary by more than the factor of about 4 when compared to the non-polar solvent to drug ratio of the selected reference formulation, will be bioequivalent to the selected reference formulation and other family members, when dosed at the same total weight of paricalcitol (i.e., containing the same total weight of paricalcitol). The oral formulations of the present invention having the above-described non-polar solvent to paricalcitol ratios will have the same kinetic profile and will result in a patient receiving the same bioavailable fraction of paricalcitol, regardless of the dosage strength ingested (i.e., see Example 1).

As can be appreciated by those skilled in the art, the ability to deliver the oral formulations of the present invention to subjects in need of treatment (i.e, pre-end stage and end-stage CKD patients) in a variety of different dosage strengths (for example, 0.25 mcg, 0.50 mcg, 1.0 mcg, 2.0 mcg, 4.0 mcg, 16.0 mcg or 32.0 mcg) without impacting the pharmacokinetic profile or the bioequivalency is very important. More specifically, the ability to treat a patient with a variety of dosage forms of paricalcitol that are bioequivalent allows a physician to treat patients in an appropriate manner. For example, a physician can use the oral formulations of the present invention in various dosage forms to minimize the cycling between over-suppression of iPTH by administering more drug than is needed by a patient at a particular time in his treatment regimen as iPTH levels fall and under-suppression by administering insufficient levels of drug than needed as iPTH levels rebound.

As mentioned above, any ratio of non-polar solvent (in milligrams) to paricalcitol (in micrograms) that is desired can be used in the selected reference formulation, the design or determination of which is well within the skill of one of ordinary skill in the art. Factors that can be considered in designing or determining the amount of non-polar solvent to paricalcitol ratio include solubility of the paricalcitol and convenience of the size of the resulting dosage form for patients.

Key to the present invention is the discovery that, once the ratio of solvent to paricalcitol for the selected reference formulation has been designed/determined, additional formulations of varying dosage strengths can be prepared straightforwardly, provided that said ratio does not vary by more than a factor of about 4 from the ratio of the selected reference formulation. Thus, the non-polar solvent to drug ratio need not be identical for all varying dosage strengths. In other words, according to the invention, as long as the non-polar solvent to drug ratio in a given formulation does not vary by more than a factor of about 4 from that in the selected reference formulation, one can obtain a family of bioequivalent dosage forms even though the non-polar solvent to drug ratio in specific family members may vary by more than a factor of about 4 from each other. Thus, only the amounts of non-polar solvent and drug relative to each other matter in developing bioequivalent dosage forms. The present invention therefore contrasts with the March 2003 FDA Guidance which teaches that all ingredients, including excipients, must be varied proportionally to achieve bioequivalence between dosage forms.

By way of illustration, and not of limitation, the following example of how oral formulations of varying dosage strengths that are bioequivalent to one another, when dosed at the same total weight of paricalcitol, can be prepared as described herein shall now be given. An oral formulation containing about 2.0 mcg of paricalcitol can be dissolved in about 140.56 mg of a non-polar solvent using routine techniques in the art. For purposes of comparison and elucidating the invention, this formulation is deemed to be the “selected reference formulation”. In this selected reference formulation, the ratio of at least one non-polar solvent to paricalcitol is about 140.56:2.0 or about 70.28:1.0. By utilizing this ratio, other oral formulations of varying dosage strengths can be made that are bioequivalent to this selected reference formulation. Other excipients, such as encapsulation agents, play little, if any, role in the overall bioavailability and can be scaled in a disproportionate manner to accommodate, for example, manufacturing considerations.

For example, an oral formulation of about 1.0 mcg of paricalcitol can be dissolved in about 70.28 mg of non-polar solvent using routine techniques known in the art. Because the non-polar solvent to drug ratio is less than a factor of about 4 when compared to the selected reference formulation, this 1.0 mcg formulation is bioequivalent to the selected reference formulation.

By way of another example, an oral formulation containing about 0.50 mcg paricalcitol can be dissolved in about 35.14 mg of non-polar solvent using routine techniques known in the art. In this example, it would be extremely difficult following conventional guidances to proportionally scale certain excipients, such as, but not limited to, gelatin, in a manner to yield a gelatin capsule that would be acceptable. However, the present invention allows one to readily manufacture a dosage form such as a capsule by choosing an amount of encapsulation agent that would yield a capsule that is pharmaceutically acceptable. In this example, because the non-polar solvent to drug ratio differs by less than a factor of about 4 when compared to the selected reference formulation (35.14/0.5 equals a ratio of 70.28:1.0), this 0.5 mcg formulation is bioequivalent to the selected reference formulation.

By way of another example, an oral formulation containing about 0.25 mcg paricalcitol can be dissolved in about 17.57 mg of non-polar solvent using routine techniques known in the art. Because the non-polar solvent to drug ratio is less than a factor of about 4 when compared to the selected reference formulation (17.57/0.25 equals a ratio of 70.28:1.0), this 0.25 mcg capsule formulation is bioequivalent to the selected reference formulation. Each of these formulations, containing, respectively, 1.0 mcg, 0.50 mcg and 0.25 mcg paricalcitol, is bioequivalent to one another. Thus, if, for example, a dose of 2.0 mcg paricalcitol is prescribed to a patient, the bioavailable fraction of the drug that the patient receives and its associated kinetic profile will be the same whether the patient takes one (1) 2.0 mcg capsules (i.e., the selected reference formulation), two (2) of the 1.0 mcg capsules described herein, four (4) of the 0.50 mcg capsules described herein or eight (8) of the 0.25 mcg capsules described herein.

By way of further illustration, and not of limitation, the following additional examples of how oral formulations of varying dosage strengths that are bioequivalent to one another can be prepared as described herein shall now be given.

An oral formulation containing about 4.0 mcg of paricalcitol can dissolved in about 140.56 mg of a non-polar solvent using routine techniques in the art. For purposes of comparison and elucidating the invention, this formulation is deemed to be the “selected reference formulation”. In this selected reference formulation, the ratio of at least one non-polar solvent to paricalcitol is about 140.56:4.0 or about 35.14:1.0. By utilizing this ratio, other oral formulations of varying dosage strengths can be made that are bioequivalent to this selected reference formulation. For example, an oral formulation of about 1.0 mcg of paricalcitol can be dissolved in about 35.14 mg of non-polar solvent using routine techniques known in the art. Because the non-polar solvent to drug ratio is less than a factor of about 4 when compared to the selected reference formulation, this 1.0 mcg dosage form is considered to be bioequivalent to the selected reference formulation.

By way of another example, an oral formulation containing about 0.50 mcg paricalcitol can be dissolved in about 11.71 mg of non-polar solvent using routine techniques known in the art. Because the non-polar solvent to drug ratio is less than a factor of about 4 when compared to the selected reference formulation (11.71/0.50 equals a ratio of 23.43:1.0), this 0.5 mcg dosage form is bioequivalent to the selected reference formulation, i.e., the 4 mcg formulation described above

By way of another example, an oral formulation containing about 0.25 mcg paricalcitol can be dissolved in about 8.78 mg of non-polar solvent using routine techniques known in the art. Because the non-polar solvent to drug ratio is less than a factor of about 4 when compared to the selected reference formulation (8.78/0.25 equals a ratio of 35.12:1.0), this 0.25 mcg dosage form is bioequivalent to the selected reference formulation. Each of these formulations, containing, respectively, 4.0 mcg, 1.0 mcg, 0.50 mcg and 0.25 mcg paricalcitol, are bioequivalent to one another, even though the non-polar solvent to drug ratio is not the same for all family members. That is, the non-polar solvent: drug ratios for each of these family members is: for 4 mcg formulation=35.14: 1.0; for 1 mcg formulation=35.14: 1.0; for 0.5 mcg formulation=23.43: 1.0; and for 0.25 mcg formulation=35.12:1.0.

By way of yet further illustration, and not of limitation, the following additional examples of how oral formulations of varying dosage strengths that are bioequivalent to one another can be prepared as described herein shall now be given. An oral formulation containing about 4.0 mcg of paricalcitol can be dissolved in about 140.56 mg of a non-polar solvent using routine techniques in the art. For purposes of comparison and elucidating the invention, this formulation is deemed to be the “selected reference formulation”. In this selected reference formulation, the ratio of at least one non-polar solvent to paricalcitol is about 140.56:4.0 or about 35.14:1.0. By utilizing this ratio, other oral formulations of varying dosage strengths can be made that are bioequivalent to this selected reference formulation.

For example, an oral formulation of about 16.0 mcg of paricalcitol can be dissolved in about 562.24 mg of non-polar solvent using routine techniques known in the art. Because the non-polar solvent to drug ratio is less than a factor of about 4 when compared to the selected reference formulation, this 16.0 mcg formulation is bioequivalent to the selected reference formulation.

By way of another example, an oral formulation containing about 32.0 mcg paricalcitol can be dissolved in about 1124.48 mg of non-polar solvent using routine techniques known in the art. Because the non-polar solvent to drug ratio is less than a factor of about 4 when compared to the selected reference formulation, this 32.0 mcg formulation is bioequivalent to the selected reference formulation. Each of these formulations, containing, respectively, 4.0 mcg, 16.0 mcg and 32.0 mcg of paricalcitol, are bioequivalent to one another.

The oral formulations of the present invention are not limited to any single type of dosage form having any particular mechanism of drug release. Thus, for example, tablets, and hard capsules and soft gelatin capsules are within the scope of the invention. The above-described beneficial bioequivalency can be obtained with any of the oral release dosage forms in use today. These dosage forms and the techniques for making them are well-known to those skilled in the art.

An example of three (3) commonly used oral polymeric controlled release dosage forms, includes matrix systems, osmotic pumps, and membrane controlled technology (also referred to as reservoir systems). Each of these systems is described in greater detail below. A detailed discussion of such dosage forms may also be found in: (i) Handbook of Pharmaceutical Controlled Release Technology, ed. D. L. Wise, Marcel Dekker, Inc. New York, N.Y. (2000), and (ii) Treatise on Controlled Drug Delivery, Fundamentals, Optimization, and Applications, ed. A. Kydonieus, Marcel Dekker, Inc. New York, N.Y. (1992), the contents of each which is hereby incorporated by reference.

A) Matrix Systems

Matrix systems are well known in the art. In a matrix system, the paricalcitol and non-polar solvent are homogenously dispersed in a polymer in association, and optionally, with additional excipients, alcohols, etc. This admixture is typically compressed under pressure to produce a tablet. Paricalcitol is released from this tablet by diffusion and erosion. Matrix systems are described in detail by Wise and Kydonieus, supra.

The matrix formulations of this invention comprise paricalcitol, a non-polar solvent and a pharmaceutically acceptable polymer. The pharmaceutically acceptable polymer is a water-soluble hydrophilic polymer, or a water insoluble hydrophobic polymer (or nonpolymeric). Examples of suitable water soluble polymers include polyvinylpyrrolidine, hydroxypropylcellulose, hydroxypropylmethyl cellulose, methyl cellulose, vinyl acetate copolymers, polysaccharides (such as alignate, xanthum gum, etc.), polyethylene oxide, methacrylic acid copolymers, maleic anhydride/methyl vinyl ether copolymers and derivatives and mixtures thereof. Examples of suitable water insoluble polymers include acrylates, cellulose derivatives such ethylcellulose or cellulose acetate, polyethylene, methacrylates, acrylic acid copolymers and high molecular weight polyvinylalcohols. Examples of suitable waxes include fatty acids and glycerides.

Preferably, the polymer is selected from hydroxypropyl cellulose, hydroxypropylmethyl cellulose, and methyl cellulose. More preferably, the polymer is hydroxypropylmethyl cellulose. Most preferably, the polymer is a high viscosity hydroxypropyl-methyl cellulose with viscosity ranging from about 4,000 cps to about 100,000 cps. The most preferred high viscosity polymer is a hydroxypropylmethyl cellulose with a viscosity of about 15,000 cps, commercially available under the tradename, Methocel, from The Dow Chemical Company.

The formulation of the present invention can also include additional pharmaceutically acceptable excipients. As is well known to those skilled in the art, pharmaceutical excipients are routinely incorporated into solid dosage forms. This is done to ease the manufacturing process as well as to improve the performance of the dosage form. Common excipients include diluents or bulking agents, lubricants, binders, antioxidants, etc. Examples of antioxidants that can be used include, but are not limited to, butylated hydroxytoluene, alpha tocopherol, ascorbic acid, ascorbyl palmitate, butylated hydroxyanisole, propyl gallate, sodium ascorbate, or sodium metabisulfite.

Diluents or fillers can be added in order to increase the mass of an individual dose to a size suitable for tablet compression. Suitable diluents include powdered sugar, calcium phosphate, calcium sulfate, microcrystalline cellulose, lactose, mannitol, kaolin, sodium chloride, dry starch, sorbitol, etc.

Lubricants can be incorporated into a formulation for a variety of reasons. They reduce friction between the granulation and die wall during compression and ejection. This prevents the granulate from sticking to the tablet punches, facilitates its ejection from the tablet punches, etc. Examples of suitable lubricants include talc, stearic acid, vegetable oil, calcium stearate, zinc stearate, magnesium stearate, etc.

Glidants can also be incorporated into the formulation. A glidant improves the flow characteristics of the granulation. Examples of suitable glidant's include talc, silicon dioxide, and cornstarch.

Binders may be incorporated into the formulation. Binders are typically utilized if the manufacture of the dosage form uses a granulation step. Examples of suitable binders include pyrrolidone, polyvinylpyrrolidone, xanthan gum, cellulose gums such as carboxymethylcellulose, methyl cellulose, hydroxypropylmethylcellulose, hydroxycellulose, gelatin, starch, and pregelatinized starch.

Other excipients that may be incorporated into the formulation include preservatives, antioxidants, or any other excipient commonly used in the pharmaceutical industry, etc. The amount of excipients used in the formulation will correspond to that typically used in a matrix system and do not need to be confined to the ratios described earlier with respect to the non-polar solvent to drug (paricalcitol) ratio.

Additionally, at least one low molecular weight alcohol can also be used in the formulation.

The matrix formulations are generally prepared using standard techniques well known in the art. Typically, they are prepared by dry blending the polymer, a non-polar solvent, paricalcitol, and other excipients, fillers and antioxidants followed by granulating the mixture using an alcohol until proper granulation is obtained. The granulation is done by methods known in the art. The wet granules are dried in a fluid bed dryer, sifted and ground to appropriate size. Lubricating agents are mixed with the dried granulation to obtain the final formulation.

For example, the formulations of the invention can be administered orally in the form of a solution or syrup, as tablets or pills, or can be loosely filled into capsules (hard or soft). Tablets can be prepared by techniques known in the art and contain a therapeutically useful amount of the paricalcitol and at least one non-polar solvent as is necessary to form the tablet by such techniques. Tablets and pills can additionally be prepared with enteric coatings and other release-controlling coatings for the purpose of acid protection, easing swallow ability, etc. The coating may be colored with a pharmaceutically accepted dye. The amount of dye and other excipients in the coating liquid may vary and will not impact the performance of the extended release tablets. The coating liquid generally comprises film forming polymers such as hydroxypropyl cellulose, hydroxypropylmethyl cellulose, cellulose esters or ethers (such as cellulose acetate or ethylcellulose), an acrylic polymer or a mixture of polymers. The coating solution is generally an aqueous solution or an organic solvent further comprising propylene glycol, sorbitan monoleate, sorbic acid, fillers such as titanium dioxide and a pharmaceutically acceptable dye.

An example of a soft capsule that can be used is a soft elastic gelatin capsule. The composition of a soft elastic gelatin capsule typically comprises from about 30% to about 50% by weight of gelatin NF, from about 10% to about 40% by weight of a plasticizer or a blend of plasticizers and from about 25% to about 40% by weight of water. Plasticizers useful in the preparation of soft elastic gelatin capsules are glycerin, sorbitol or sorbitol derivatives (i.e, sorbitol-special and the like) or propylene glycol and the like; or combinations thereof. The soft elastic gelatin capsule material can also comprise additives such as preservatives, opacifiers, pigments, dyes or flavors and the like.

Various methods can be used for manufacturing and filling the soft elastic gelatin capsules, for example, a seamless capsule method, a rotary method (developed by Scherer) or a method using a Liner machine or an Accogel machine and the like. Methods for manufacturing soft gelatin capsules are described in U.S. Pat. Nos. 4,744,988 and 5,985,321, herein incorporated by reference. Various manufacturing machines can be used for manufacturing capsules. Typically, the soft elastic gelatin capsule is prepared by (1) preparing the gel mass, (2) encapsulating the fill material (forming, filling and sealing the capsule) and (3) softgel drying. During gel mass preparation, the ingredients comprising the gel mass (typically, gelatin, water and plasticizer) are mixed to form a uniform fluff. After blending, the fluff gel mass is melted, preferably, under vacuum, and the melted gel mass is transferred to heated receivers. Colorants or other additives can be added to the melted gel mass, which is then blended until uniform.

In one method, a rotary die encapsulation apparatus is then used to encapsulate the liquid capsule fill. In general, in this method two gel ribbons are fed between two rotating dies. The dies contain paired pockets, which form the shape of the softgel and provide the sealing mechanism. At the moment the two die half pockets line up, the fill material is injected through an encapsulation wedge in between the gel ribbons. The softgel is formed and sealed as a result of pressure between the dies and heat applied by the encapsulation wedge. Finally, the filled softgels are dried. In one method, the filled softgels are first placed in a rotary drier in a low humidity, forced air environment. A final step in the drying process involves discharging the filled softgels from the rotary drier and placing them in a monolayer on shallow drying trays, over which is circulated low humidity air of less than 50% relative humidity. The drying process is stopped by transferring the softgels into deep holding trays.

A variety of hard gelatin capsules are known in the art. For example, hard gelatin capsules can be purchased from Capsugel, Greenwood, S.C. and other suppliers. Capsules are filled manually or by capsule filling machine. The target filling volume/weight depends on the potency of the filling solution in combination with the desired dosage strength.

A particularly preferred matrix system for oral formulation of the present invention comprises a mixture of from about 0.25 to about 32.0 mcg paricalcitol, from about 1.0 to about 3500.0 mg of a non-polar solvent and optionally, at least one antioxidant. This mixture can then be encapsulated in an amount of a suitable matrix that provides a pharmaceutically acceptable oral dosage form. Such a suitable matrix includes, but is not limited to, soft gelatin, hard gelatin, hydroxylpropyl methyl cellulose, and polymethacrylates. If a soft gelatin capsule is used, this capsule can have a fill weight of from about 17.0 mg to about 2250 mg.

B) Osmotic Pumps

In an osmotic pump system, a tablet core is encased by a semipermeable membrane having at least one orifice. The semipermeable membrane is permeable to water, but impermeable to the drug. When the system is exposed to body fluids, water will penetrate through the semipermeable membrane into the tablet core containing osmotic excipients and the active drug. Osmotic pressure increases within the dosage form and drug is released through the orifice in an attempt to equalize pressure.

In more complex pumps, the tablet core contains two internal compartments. The first compartment contains the drug. The second compartment contains a polymer which swells on contact with fluid. After ingestion, this polymer swells into the drug containing compartment at a predetermined rate and forces drug from the dosage form at that rate. Such dosage forms are often used when are zero order release profile is desired.

Osmotic pumps are well known in the art and have been described in the literature. U.S. Pat. Nos. 4,088,864, 4,200,098 and 5,573,776, all of which are hereby incorporated by reference, describe osmotic pumps and methods for their manufacture. One skilled in the art, taking into account this application and the teachings and those of the U.S. Pat. Nos. 4,088,864, 4,200,098 and 5,573,776 could produce an osmotic pump matching the pharmacokinetic profile described above.

As a general guideline, the osmotic pumps of this invention are typically formed by compressing a tablet of an osmotically active drug (or an osmotically inactive drug in combination with an osmotically active agent or osmagent) and then coating the tablet with a semipermeable membrane which is permeable to an exterior aqueous-based fluid but impermeable to the passage of drug and/or osmagent. One or more delivery orifices may be drilled through the semipermeable membrane wall. Alternatively, orifice(s) through the wall may be formed in situ by incorporating leachable pore forming materials in the wall. In operation, the exterior aqueous based fluid is imbibed through the semipermeable membrane wall and contacts the drug and/or salt to form a solution or suspension of the drug. The drug solution or suspension is then pumped out through the orifice as fresh fluid is imbibed through the semipermeable membrane.

In a further embodiment, the tablet contains two distinct compartments. The first compartment contains the drug as described above. The second compartment contains an expandable driving member consisting of a layer of a swellable hydrophilic polymer, which operates to diminish the volume occupied by the drug, thereby delivering the drug from the device at a controlled rate over an extended period of time.

Typical materials for the semipermeable membrane include semipermeable polymers known to the art as osmosis and reverse osmosis membranes, such as cellulose acylate, cellulose diacylate, cellulose triacylate, cellulose acetate, cellulose diacetate, cellulose triacetate, agar acetate, amylose triacetate, beta glucan acetate, acetaldehyde dimethyl acetate, cellulose acetate ethyl carbamate, polyamides, polyurethanes, sulfonated polystyrenes, cellulose acetate phthalate, cellulose acetate methyl carbamate, cellulose acetate succinate, cellulose acetate dimethyl aminoacetate, cellulose acetate ethyl carbamate, cellulose acetate chloroacetate, cellulose dipalmitate, cellulose dioctanoate, cellulose dicaprylate, cellulose dipentanlate, cellulose acetate valerate, cellulose acetate succinate, cellulose propionate succinate, methyl cellulose, cellulose acetate p-toluene sulfonate, cellulose acetate butyrate, cross-linked selectively semipermeable polymers formed by the coprecipitation of a polyanion and a polycation as disclosed in U.S. Pat. Nos. 3,173,876, 3,276,586, 3,541,005, 3,541,006, and 3,546,142, semipermeable polymers as disclosed by Loeb and Sourirajan in U.S. Pat. No. 3,133,132, lightly cross-linked polystyrene derivatives, cross-linked poly(sodium styrene sulfonate), poly(vinylbenzyltrimethyl ammonium chloride), cellulose acetate having a degree of substitution up to 1 and an acetyl content up to 50%, cellulose diacetate having a degree of substitution of 1 to 2 and an acetyl content of 21 to 35%, cellulose triacetate having a degree of substitution of 2 to 3 and an acetyl content of 35 to 44.8%, as disclosed in U.S. Pat. No. 4,160,020.

The osmotic agent present in the pump, which may be used when the drug itself is not osmotically active, are osmotically effective compounds soluble in the fluid that enters the device, and exhibits an osmotic pressure gradient across the semipermeable wall against the exterior fluid. Osmotically effective osmagents useful for the present purpose include magnesium sulfate, calcium sulfate, magnesium chloride, sodium chloride, lithium chloride, potassium sulfate, sodium carbonate, sodium sulfite, lithium sulfate, potassium chloride, sodium sulfate, d-mannitol, urea, sorbitol, inositol, raffinose, sucrose, glucose, hydrophilic polymers such as cellulose polymers, mixtures thereof, and the like. The osmagent is usually present in an excess amount, and it can be in any physical form, such as particle, powder, granule, and the like. The osmotic pressure in atmospheres of the osmagents suitable for the invention will be greater than zero and generally up to about 500 atm, or higher.

The expandable driving member is typically a swellable, hydrophilic polymer which interacts with water and aqueous biological fluids and swells or expands to an equilibrium state. The polymers exhibit the ability to swell in water and retain a significant portion of the imbibed water within the polymer structure. The polymers swell or expand to a very high degree, usually exhibiting a 2 to 50 fold volume increase. The polymers can be noncross-linked or cross-linked. The swellable, hydrophilic polymers are in one embodiment lightly cross-linked, such cross-links being formed by covalent ionic bonds or hydrogen bonds. The polymers can be of plant, animal or synthetic origin. Hydrophilic polymers suitable for the present purpose include poly(hydroxy alkyl methacrylate) having a molecular weight of from 30,000 to 5,000,000; kappa carrageenan, polyvinylpyrrolidone having molecular weight of from 10,000 to 360,000; anionic and cationic hydrogels; polyelectrolyte complexes; poly(vinyl alcohol) having a low acetate residual, cross-linked with glyoxal, formaldehyde, or glutaraldehyde and having a degree of polymerization from 200 to 30,000; a mixture of methyl cellulose; cross-linked agar and carboxymethyl cellulose; a water insoluble, water swellable copolymer produced by forming a dispersion of finely divided copolymer of maleic anhydride with styrene, ethylene, propylene, butylene or isobutylene cross-linked with from 0.001 to about 0.5 moles of saturated cross-linking agent per mole of maleic anhydride in copolymer; water swellable polymers of N-vinyl lactams, and the like.

The expression “orifice” as used herein comprises means and methods suitable for releasing the drug from the system. The expression includes one or more apertures or orifices which have been bored through the semipermeable membrane by mechanical procedures. Alternatively it may be formed by incorporating an erodible element, such as a gelatin plug, in the semipermeable membrane. In cases where the semipermeable membrane is sufficiently permeable to the passage of drug, the pores in the membrane may be sufficient to release the agent/drug in therapeutically effective amounts. In such cases, the expression “passageway” refers to the pores within the membrane wall even though no bore or other orifice has been drilled there through. A detailed description of osmotic passageways and the maximum and minimum dimensions for a passageway are disclosed in U.S. Pat. Nos. 3,845,770 and 3,916,899, the disclosures of which are incorporated herein by reference.

The osmotic pumps of this invention are manufactured by standard techniques. For example, in one embodiment, the drug and other ingredients that may be housed in one area of the compartment adjacent to the passageway, are pressed into a solid possessing dimension that corresponds to the internal dimensions of the area of the compartment the agent will occupy, or the agent and other ingredients and a solvent are mixed into a solid or semisolid form by conventional methods such as ballmilling, calendaring, stirring or rollmilling, and then pressed into a preselected shape. Next, a layer of a hydrophilic polymer is placed in contact with the layer of agent in a like manner, and the two layers surrounded with a semipermeable wall. The layering of agent formulation and hydrophilic polymer can be fabricated by conventional two-layer press techniques. The wall can be applied by molding, spraying or dipping the pressed shapes into a wall forming material. Another and presently preferred technique that can be use for applying the wall is the air suspension procedure. This procedure consists of suspending and tumbling the pressed agent and dry hydrophilic polymer in a current of air and a wall forming composition until the wall is applied to the agent-hydrophilic polymer composite. The air suspension procedure is described in U.S. Pat. No. 2,799,241; J. Am. Pharm. Assoc., (48):451-459, (1979). Other standard manufacturing procedures are described in Modern Plastics Encyclopedia, 46:62-70 (1969); and in Pharmaceutical Sciences, by Remington, Fourteenth Edition, pp. 1626-1678 (1970), published by Mack Publishing Company, Easton, Pa.

C) Reservoir Polymeric Systems

Reservoir systems are well known in the art. This technology is also commonly referred to as microencapsulation, bead technology or coated tablets. Small particles of the drug are encapsulated with pharmaceutically acceptable polymer. This polymer, and its relative quantity, offers a predetermined resistance to drug diffusion from the reservoir to the gastrointestinal tract. Thus drug is gradually released from the beads into the gastrointestinal tract and provides the desired release of paricalcitol.

These dosage forms are well known in the art. Specifically, U.S. Pat. Nos. 5,286,497 and 5,737,320, both of which are hereby incorporated by reference, describe such formulations and their methods of production. U.S. Pat. Nos. 5,354,556, 4,952,402, and 4,940,588, all of which are hereby incorporated by reference, specifically discuss using such technology to produce sustained release dosage forms of paricalcitol. One skilled in the art, taking into account the teaching of this application and those of the U.S. Pat. Nos. 5,286,497, 5,737,320, 5,354,556, 5,952,402 and could produce a bead or pellet based dosage form matching the pharmacokinetic profile described herein.

As a general guideline however, a pellet is formed with a core containing paricalcitol and a non-polar solvent. This core is then coated with one, or more, pharmaceutically acceptable polymers. Often, the coating polymer is an admixture of a major proportion of a pharmaceutically acceptable water insoluble polymer and a minor proportion of a pharmaceutically acceptable water soluble polymer. The central core may be prepared by a number of techniques known in the art. Typically the paricalcitol is bound to an inert carrier with a conventional binding agent. The inert carrier is typically a starch or sugar sphere. Before the paricalcitol is bound to the inert carrier, it may be dissolved in a volatile polar solvent. Optionally, additional excipients, antioxidants and at least one alcohol can also be added. These excipients and alcohols are identical to those described above for the matrix systems. The quantity of these excipients and alcohols can vary widely, but will be used in conventional amounts. The central core is then produced by utilizing a binding agent to attach the paricalcitol/non-polar solvent blend to the solid carrier. This can be accomplished by means known in the art for producing pharmaceutical beads. Suitable means include utilization of a conventional coating pan, an automatic coating machine, or a roto granulator. The production of these central cores is described in more detail in Pharmaceutical Pelletization Technology, ed. I. Ghebre-Sellassie, Marcel Dekker, Inc. New York, N.Y. (1989) which is hereby incorporated by reference.

The second major component of the beads is the polymeric coating. As noted above, the polymeric coating is responsible for giving the beads their sustained release characteristics. The polymeric coating may be applied to the central core using methods and techniques known in the art. Examples of suitable coating devices include fluid bed coaters, pan coaters, etc. The application techniques are described in more detail in: 1) Aqueous Polymeric Coatings for Pharmaceutical Dosage Forms, ed. J. W. McGinity, Marcel Dekker, Inc. New York, N.Y. (1997); and 2) Pharmaceutical Dosage Forms: Tablets, Vol. 3. ed. H. A. Lieberman, L. Lachman and J. B. Schwartz, Marcel Dekker, Inc. New York, N.Y. pp. 77-287, (1990), the contents of each which are hereby incorporated by reference.

Examples of suitable polymers include ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), poly(ethylene), poly(ethylene) low density, poly(ethylene) high density, poly(propylene), poly(ethylene oxide), poly(ethylene terephthalate), poly(vinyl isobutyl ether), poly(vinyl acetate), poly(vinyl chloride) or polyurethane or mixtures thereof.

Once the beads have been prepared, they may be filled into capsules as is known in the art. Alternately, they may be pressed into tablets using techniques conventional in the art.

III. Pharmacokinetic Profile, Dosing, Bone Markers and Urinary Calcium Pharmacokinetic Profile

The oral formulations of the present invention have overall exposure very similar to that of intravenously administered paricalcitol. More particularly, the oral formulations of the present invention have a profile that provides equivalent clinical utility to intravenously administered paricalcitol. More specifically, as shown in Example 3, the oral formulations of the present invention produce mean AUC_{0-∞} value that is 80% of the mean AUC_{0-∞} value generated by intravenous administration of paricalcitol to end-stage CKD patients and produce equivalent clinical responses.

Because the oral formulations of the present invention have overall exposure very similar to that of intravenously administered paricalcitol, the oral formulations of the present invention exhibit equivalent clinical utility in terms of safety and efficacy to that exhibited by intravenously administered paricalcitol when administered to end-stage CKD patients. For example, 95% confidence intervals were calculated for the difference in proportions in clinically meaningful efficacy (30% reductions in PTH) and clinically meaningful safety (hypercalcemia) between patients receiving an intravenous formulation and those receiving oral paricalcitol formulations would capture zero. Therefore, the oral formulations of the present invention possess a number of benefits not exhibited by other orally administered and commercially available vitamin D compounds known in the art, such as Rocaltrol® (calcitriol) and Hectorol® (doxercalciferol). More specifically, the inventors of the present invention have unexpectedly discovered that the safety profile (i.e., the number of incidences of hypercalcemia and hyperphosphatemia) exhibited by end-stage CKD patients receiving the oral formulations of the present invention is not statistically significantly different than those same patients receiving intravenously administered paricalcitol. The oral formulations of the present invention are effective, in a variety of different dose strengths, in suppressing elevated levels of intact parathyroid hormone in pre-end stage and end stage CKD patients.

Despite the fact that the oral formulations of the present invention exhibit mean AUC_{0-∞} values similar to the mean AUC_{0-∞} values of intravenously administered paricalcitol in end-stage CKD patients, the oral formulations of the present invention do exhibit a statistically significant different Cₘₐₓ values when compared to intravenously administered paricalcitol in end-stage CKD patients. Although the Cₘₐₓ for the oral formulations of the present invention and intravenously administered paricalcitol are statistically significantly different, the inventors of the present invention do not believe that this difference in Cₘₐₓ affects the equivalence in clinical utility of the oral formulations of the present invention when compared to intravenously administered paricalcitol since the oral formulations of the present invention and intravenously administered paricalcitol exhibit a similar therapeutic profile in terms of safety and efficacy when administered to end-stage CKD patients. Therefore, the data (as shown in Example 3) suggests that Cₘₐₓ is not contributing to the therapeutic profile of either the oral formulations of the present invention or the intravenously administered paricalcitol when administered to end-stage CKD patients.

Dosing

For end-stage CKD patients, the standard dosing of vitamin D compounds for the treatment of 2° HPT is every other day, three times a week. This dosing regimen produces higher blood concentration and enhances PTH suppression, while minimizing the effect on calcium and phosphorus load.

The inventors of the present invention have found that, in pre-end stage renal disease patients, the oral formulations of the present invention can be dosed daily in a variety of different dosing strengths. Daily dosage of the oral formulations of the present invention was found to be equally effective and safe in preventing and treating 2° HPT (by reducing the levels of parathyroid hormone levels) in pre-end stage and end stage CKD patients as TIW dosing. The ability to dose the oral formulations of the present invention daily or three times a week provides greater convenience to the patient. If the oral formulations of the present invention are dosed on a daily basis, the strength of the dose can be 0.25 mcg, 0.5 mcg, 1.0 mcg, 2.0 mcg, 3.0 mcg or 4.0 mcg, for example. If the oral formulations of the present invention are dosed three times a week, the strength of the dose can be 2.0 mcg, 3.0 mcg, 4.0 mcg, 8.0 mcg, 16.0 mcg, or 32.0 mcg, for example. Using these dosage strengths, the average weekly dose for both daily and three times a week dosing regimens are equivalent from a safety and efficacy perspective (that is, have clinically equivalent utility). Although both dosing regimens are equivalent, daily dosing is recommended to enhance patient compliance.

Bone Markers and Bone Formation

The inventors of the present invention have discovered that CKD Stage 3 and 4 subjects receiving the oral formulations of the present invention demonstrate bone formation and that the quality of said bone formation and correction of high-turnover bone diseases is associated with 2° HPT. Serum bone-specific alkaline phosphate and serum osteocalcin are considered more sensitive bone markers to evaluate degree of bone remodeling than urinary bone marker. The statistically significant difference observed in serum bone alkaline phosphate, serum osteocalcin and urinary pyridinoline using ANOVA and ANCOVA with treatment as a factor suggest correction of high-turnover bone disease associated with 2° HPT. As shown in Examples 5-7 and Tables C (Example 5), Table D (Example 6) and Table E (Example 7), certain biochemical bone activity marker variables were analyzed in CKD Stage 3 and 4 subjects. These subjects received treatment with the oral formulations of the present invention or a placebo. The bone markers examined in these subjects were serum osteocalcin, serum bone-specific alkaline phosphatase, urinary pyridinoline, and deoxypyridinoline. If more than 1 biochemical bone activity marker measurement existed for a subject on a particular day, the higher measurement was considered to be that subject's biochemical bone activity marker measurement for that day. The baseline for biochemical bone activity markers was defined as the last biochemical bone activity marker measurement collected on or before the date the first dose of study drug was taken. The Final Visit measurement was defined as the last biochemical bone activity marker measurement following the first dose of study drug. Subjects who did not have a baseline and a Final Visit measurement were not included in Final Visit analyses.

The Week 11 Visit measurement was defined as the biochemical bone activity marker measurement on the day closest to the Week 11 scheduled visit, for which the possible measurements to choose from were those collected within 64 and 77 days following the first dose of study drug. Subjects who did not have both a baseline and a Week 11 measurement were not included in Week 11 analyses.

Changes from baseline to Week 11 Visit and to Final Visit in biochemical bone activity markers were compared between oral paricalcitol and placebo using an ANOVA with treatment as the factor on the combined Phase 3 all treated subject population (Table F). Also, the changes from baseline to Week 11 Visit and to Final Visit were compared between oral paricalcitol and placebo using an ANCOVA with baseline as the second factor on the combined Phase 3 all treated subject population (Table G).

Statistically significant differences were observed between the oral paricalcitol and placebo treatment groups in mean change from baseline to Week 11 in the biochemical bone activity markers of serum bone-specific alkaline phosphatase and urinary deoxypyridinoline using ANOVA with treatment as the factor. The oral paricalcitol group had a greater mean decrease (−5.024 mcg/L) from baseline in serum bone-specific alkaline phosphatase compared to a small decrease observed in the placebo group (−1.749 mcg/L). Additionally, the oral paricalcitol group had a mean decrease in urinary deoxypyridinoline (−0.0155 nmol/mg Creat), while the placebo group experienced a mean increase (0.0024 nmol/mg Creat). No statistically significant differences were observed between the oral paricalcitol and placebo treatment groups in mean change from baseline to Week 11 in serum osteocalcin and urinary pyridinoline using ANOVA with treatment as the factor. Results were similar using ANCOVA with treatment as the factor and baseline value as the covariate.

The differences between the treatment groups in mean change from baseline to Final Visit in the biochemical bone activity markers of serum bone-specific alkaline phosphatase, serum osteocalcin, and urinary pyridinoline were statistically significant using ANOVA with treatment as the factor. The oral paricalcitol experienced mean decreases in these biochemical bone activity markers (−7.89 mcg/L, −21.64 ng/mL, and −3.61 nmol/mmol Creat, respectively), while the placebo group experienced a small mean decrease in serum bone-specific alkaline phosphatase (−1.444 mcg/L) and mean increases in serum osteocalcin (10.74 ng/mL) and urinary pyridinoline (3.77 nmol/mmol Creat). No statistically significant difference was observed between the treatment groups in mean change from baseline to Final Visit in urinary deoxypyridinoline using ANOVA with treatment as the factor. Results were similar using ANCOVA with treatment as the factor and baseline value as the covariate.

Serum bone-specific alkaline phosphatase and osteocalcin are currently considered more sensitive and specific bone markers to evaluate the degree of bone remodeling in the setting of CKD than urine bone markers. The favorable result observed using the oral paricalcitol formulations of the present invention suggest correction of high-turnover bone disease associated with 20 HPT.

The mean changes from baseline to Week 11 and Final Visit in biochemical bone activity marker variables for the combined data are summarized by treatment group in Table F.

TABLE C
Mean Change from Baseline to Week 11 and Final Visit in Biochemical
Bone Activity Marker Variables in Example 5 (TIW Treated Subjects)

| | | |
|---|---|---|
| | | ANOVA |
| Oral Paricalcitol | Placebo | P-Value^{a} |

Serum Bone-Specific Alkaline Phosphatase (mcg/L)

| | | | |
|---|---|---|---|
| Number of Subjects | 31 | 30 | |
| Mean Baseline Value | 15.971 | 21.450 | |
| Change from Baseline (SE) to Week 11 | −4.673 (1.2590) | −1.462 (1.2798) | 0.079 |
| Number of Subjects | 36 | 35 | |
| Mean Baseline Value | 16.317 | 22.014 | |
| Change from Baseline (SE) to Final | −7.922 (1.3520) | −2.278 (1.3712) | 0.005 |

Serum Osteocalcin (ng/mL)

| | | | |
|---|---|---|---|
| Number of Subjects | 32 | 27 | |
| Mean Baseline Value | 57.91 | 78.00 | |
| Change from Baseline (SE) to Week 11 | −4.23 (4.386) | 2.47 (4.774) | 0.306 |
| Number of Subjects | 35 | 32 | |
| Mean Baseline Value | 59.70 | 81.85 | |
| Change from Baseline (SE) to Final | −19.00 (4.381) | 14.56 (4.581) | <0.001 |

Urinary Deoxypyridinoline (nmol/mg Creat)

| | | | |
|---|---|---|---|
| Number of Subjects | 32 | 27 | |
| Mean Baseline Value | 0.0500 | 0.0525 | |
| Change from Baseline (SE) to Week 11 | −0.0116 (0.00354) | 0.0026 (0.00385) | 0.009 |
| Number of Subjects | 35 | 31 | |
| Mean Baseline Value | 0.0476 | 0.0502 | |
| Change from Baseline (SE) to Final | −0.0084 (0.00375) | 0.0034 (0.00398) | 0.033 |

Urinary Pyridinoline (nmol/mmol Creat)

| | | | |
|---|---|---|---|
| Number of Subjects | 33 | 28 | |
| Mean Baseline Value | 35.92 | 36.20 | |
| Change from Baseline (SE) to Week 11 | −5.46 (2.545) | −5.86 (2.763) | 0.916 |
| Number of Subjects | 36 | 33 | |
| Mean Baseline Value | 35.47 | 34.85 | |
| Change from Baseline (SE) to Final | −4.06 (2.276) | 2.73 (2.377) | 0.043 |

^{a}One-way ANOVA with treatment as the factor.

TABLE D
Mean Change from Baseline to Week 11 or Final Visit in Biochemical
Bone Activity Marker Variables in Example 6 (TIW Treated Subjects)

| | | |
|---|---|---|
| | | ANOVA |
| Oral Paricalcitol | Placebo | p-value^{a} |

Serum Bone-Specific Alkaline Phosphatase (mcg/L)

| | | | |
|---|---|---|---|
| Number of Subjects | 29 | 32 | |
| Mean Baseline Value | 16.277 | 17.247 | |
| Change from Baseline (SE) to Week 11 | −5.078 (0.9420) | −1.448 (0.8968) | 0.007 |
| Number of Subjects | 32 | 35 | |
| Mean Baseline Value | 17.077 | 17.543 | |
| Change from Baseline (SE) to Final | −8.179 (1.2010) | −1.781 (1.1484) | <0.001 |

Serum Osteocalcin (ng/mL)

| | | | |
|---|---|---|---|
| Number of Subjects | 29 | 33 | |
| Mean Baseline Value | 55.31 | 56.12 | |
| Change from Baseline (SE) to Week 11 | −6.96 (3.259) | 3.98 (3.055) | 0.017 |
| Number of Subjects | 32 | 35 | |
| Mean Baseline Value | 56.09 | 55.44 | |
| Change from Baseline (SE) to Final | −18.92 (3.973) | 11.35 (3.799) | <0.001 |

Urinary Deoxypyridinoline (nmol/mg Creat)

| | | | |
|---|---|---|---|
| Number of Subjects | 28 | 28 | |
| Mean Baseline Value | 0.0698 | 0.0479 | |
| Change from Baseline (SE) to Week 11 | −0.0144 (0.00813) | 0.0005 (0.00813) | 0.199 |
| Number of Subjects | 31 | 33 | |
| Mean Baseline Value | 0.0682 | 0.0464 | |
| Change from Baseline (SE) to Final | −0.0182 (0.00620) | −0.0034 (0.00601) | 0.091 |

Urinary Pyridinoline (nmol/mmol Creat)

| | | | |
|---|---|---|---|
| Number of Subjects | 28 | 32 | |
| Mean Baseline Value | 38.75 | 29.81 | |
| Change from Baseline (SE) to Week 11 | −4.87 (3.725) | −0.65 (3.484) | 0.411 |
| Number of Subjects | 31 | 35 | |
| Mean Baseline Value | 38.60 | 29.17 | |
| Change from Baseline (SE) to Final | −7.11 (2.828) | 1.95 (2.662) | 0.023 |

^{a}One-way ANOVA with treatment as the factor.

TABLE E
Mean Change from Baseline to Week 11 or Final Visit in Biochemical
Bone Activity Marker Variables in Example 7 (Daily Treated Subjects)

| | | |
|---|---|---|
| | | ANOVA |
| Oral Paricalcitol | Placebo | p-value^{a} |

Serum Bone-Specific Alkaline Phosphatase (mcg/L)

| | | | |
|---|---|---|---|
| Number of Subjects | 26 | 33 | |
| Mean Baseline Value | 17.938 | 17.029 | |
| Change from Baseline (SE) to Week 11 | −5.383 (1.0482) | −2.302 (0.9304) | 0.032 |
| Number of Subjects | 33 | 37 | |
| Mean Baseline Value | 17.945 | 17.074 | |
| Change from Baseline (SE) to Final | −7.575 (1.3976) | −0.336 (1.3199) | <0.001 |

Serum Osteocalcin (ng/mL)

| | | | |
|---|---|---|---|
| Number of Subjects | 26 | 33 | |
| Mean Baseline Value | 76.07 | 76.28 | |
| Change from Baseline (SE) to Week 11 | −0.22 (4.968) | −2.35 (4.410) | 0.749 |
| Number of Subjects | 33 | 37 | |
| Mean Baseline Value | 72.29 | 76.12 | |
| Change from Baseline (SE) to Final | −27.07 (5.544) | 6.87 (5.236) | <0.001 |

Urinary Deoxypyridinoline (nmol/mg Creat)

| | | | |
|---|---|---|---|
| Number of Subjects | 26 | 33 | |
| Mean Baseline Value | 0.0834 | 0.0659 | |
| Change from Baseline (SE) to Week 11 | −0.0216 (0.01018) | 0.0039 (0.00903) | 0.066 |
| Number of Subjects | 30 | 36 | |
| Mean Baseline Value | 0.0800 | 0.0648 | |
| Change from Baseline (SE) to Final | 0.0100 (0.01392) | 0.0092 (0.01271) | 0.968 |

Urinary Pyridinoline (nmol/mmol Creat)

| | | | |
|---|---|---|---|
| Number of Subjects | 26 | 33 | |
| Mean Baseline Value | 39.61 | 37.46 | |
| Change from Baseline (SE) to Week 11 | −5.27 (3.884) | 0.52 (3.447) | 0.269 |
| Number of Subjects | 32 | 36 | |
| Mean Baseline Value | 40.10 | 37.27 | |
| Change from Baseline (SE) to Final | 0.26 (4.452) | 6.48 (4.197) | 0.313 |

^{a}One-way ANOVA with treatment as the factor.

TABLE F
Mean Change from Baseline to Week 11 and Final Visit in Biochemical Bone
Activity Marker Variables in the Double-Blind, Placebo-Controlled,
Phase 3 Studies Combined - Examples 5-7 (All Treated Subjects)

| | | |
|---|---|---|
| | | ANOVA |
| Oral Paricalcitol | Placebo | p-value^{a} |

Serum Bone-Specific Alkaline Phosphatase (mcg/L)

| | | | |
|---|---|---|---|
| Number of Subjects | 86 | 95 | |
| Mean Baseline Value | 16.669 | 18.499 | |
| Change from Baseline (SE) to Week 11 | −5.024 (0.6279) | −1.749 (0.5974) | <0.001 |
| Number of Subjects | 101 | 107 | |
| Mean Baseline Value | 17.090 | 18.843 | |
| Change from Baseline (SE) to Final | −7.890 (0.7596) | −1.444 (0.7380) | <0.001 |

Serum Osteocalcin (ng/mL)

| | | | |
|---|---|---|---|
| Number of Subjects | 87 | 93 | |
| Mean Baseline Value | 62.47 | 69.63 | |
| Change from Baseline (SE) to Week 11 | −3.94 (2.431) | 1.30 (2.351) | 0.123 |
| Number of Subjects | 100 | 104 | |
| Mean Baseline Value | 62.70 | 70.92 | |
| Change from Baseline (SE) to Final | −21.64 (2.706) | 10.74 (2.654) | <0.001 |

Urinary Deoxypyridinoline (nmol/mg Creat)

| | | | |
|---|---|---|---|
| Number of Subjects | 86 | 88 | |
| Mean Baseline Value | 0.0665 | 0.0560 | |
| Change from Baseline (SE) to Week 11 | −0.0155 (0.00433) | 0.0024 (0.00429) | 0.004 |
| Number of Subjects | 96 | 100 | |
| Mean Baseline Value | 0.0644 | 0.0542 | |
| Change from Baseline (SE) to Final | −0.0058 (0.00514) | 0.0033 (0.00504) | 0.208 |

Urinary Pyridinoline (nmol/mmol Creat)

| | | | |
|---|---|---|---|
| Number of Subjects | 87 | 93 | |
| Mean Baseline Value | 37.94 | 34.45 | |
| Change from Baseline (SE) to Week 11 | −5.21 (1.938) | −1.80 (1.875) | 0.207 |
| Number of Subjects | 99 | 104 | |
| Mean Baseline Value | 37.95 | 33.78 | |
| Change from Baseline (SE) to Final | −3.61 (1.896) | 3.77 (1.850) | 0.006 |

^{a}One-way ANOVA with treatment as the factor.

Mean Change from Baseline to Week 11 and Final Visit in Biochemical Bone Activity Marker
Variables in the Double-Blind, Placebo-Controlled, Phase 3 Studies by
Treatment Regimen - Examples 5-7 (All Treated Subjects)

| TIW Treatment Regimen | | | | |
|---|---|---|---|---|
| | | ANOVA | QD Treatment [symbol] | |
| Oral Paricalcitol | Placebo | p-value^{a} | Oral Paricalcitol | Placeb [symbol] |

Serum Bone-Specific Alkaline Phosphatase (mcg/L)

| | | | | | |
|---|---|---|---|---|---|
| Number of Subjects | 60 | 62 | | 26 | 33 |
| Mean Baseline Value | 16.119 | 19.281 | | 17.938 | 17.029 |
| Change from Baseline (SE) to Week 11 | −4.869 (0.7835) | −1.455 (0.7708) | 0.002 | −5.383 (1.0482) | −2.302 (0.9 [symbol] |
| Number of Subjects | 68 | 70 | | 33 | 37 |
| Mean Baseline Value | 16.674 | 19.779 | | 17.945 | 17.074 |
| Change from Baseline (SE) to Final | −8.043 (0.9033) | −2.030 (0.8903) | <0.001 | −7.575 (1.3976) | −0.336 (1.3 [symbol] |

Serum Osteocalcin (ng/mL)

| | | | | | |
|---|---|---|---|---|---|
| Number of Subjects | 61 | 60 | | 26 | 33 |
| Mean Baseline Value | 56.68 | 65.97 | | 76.07 | 76.28 |
| Change from Baseline (SE) to Week 11 | −5.53 (2.720) | 3.30 (2.743) | 0.024 | −0.22 (4.968) | −2.35 (4.4 [symbol] |
| Number of Subjects | 67 | 67 | | 33 | 37 |
| Mean Baseline Value | 57.98 | 68.05 | | 72.29 | 76.12 |
| Change from Baseline (SE) to Final | −18.96 (2.944) | 12.88 (2.944) | <0.001 | −27.07 (5.544) | 6.87 (5.2 [symbol] |

Urinary Deoxypyridinoline (nmol/mg Creat)

| | | | | | |
|---|---|---|---|---|---|
| Number of Subjects | 60 | 55 | | 26 | 33 |
| Mean Baseline Value | 0.0592 | 0.0501 | | 0.0834 | 0.0659 |
| Change from Baseline (SE) to Week 11 | −0.0129 (0.00426) | 0.0015 (0.00445) | 0.021 | −0.0216 (0.01018) | 0.0039 (0.0 [symbol] |
| Number of Subjects | 66 | 64 | | 30 | 36 |
| Mean Baseline Value | 0.0573 | 0.0482 | | 0.0800 | 0.0648 |
| Change from Baseline (SE) to Final | −0.0130 (0.00357) | −0.0001 (0.00362) | 0.012 | 0.0100 (0.01392) | 0.0092 (0.0 [symbol] |

Urinary Pyridinoline (nmol/mmol Creat)

| | | | | | |
|---|---|---|---|---|---|
| Number of Subjects | 61 | 60 | | 26 | 33 |
| Mean Baseline Value | 37.22 | 32.79 | | 39.61 | 37.46 |
| Change from Baseline (SE) to Week 11 | −5.19 (2.213) | −3.08 (2.232) | 0.503 | −5.27 (3.884) | 0.52 (3.4 [symbol] |
| Number of Subjects | 67 | 68 | | 32 | 36 |
| Mean Baseline Value | 36.92 | 31.93 | | 40.10 | 37.27 |
| Change from Baseline (SE) to Final | −5.47 (1.789) | 2.33 (1.776) | 0.002 | 0.26 (4.452) | 6.48 (4.1· |

^{a}Based on a one-way ANOVA with treatment as the factor.

[image]

The following examples are presented in order to further illustrate the invention.

### EXAMPLE 1

### Oral Paricalcitol Formulations, Methods of Making Said Formulations and Demonstration of Bioequivalency Among Various Formulations

The following four (4) formulations were prepared as gelatin capsules.

TABLE 1

| | | | | | Formulation | | | |
|---|---|---|---|---|---|---|---|---|
| | Formulation 1 | | Formulation 2 | | 3 Unit | | Formulation 4 | |
| | Unit Formula | | Unit Formula | | Formula | | Unit Formula | |
| Ingredients | (per Capsule) | | (per Capsule) | | (per Capsule) | | (per Capsule) | |
| Fill Solution | | | | | | | | |
| Paricalcitol | 1 | mcg | 2 | mcg | 4 | mcg | 1 | mcg |
| Dehydrated Alcohol | 1.42 | mg | 1.42 | mg | 1.42 | mg | 0.71 | mg |
| BHT | 16 | mcg | 16 | mcg | 16 | mcg | 8 | mcg |
| Neobee M-5 Oil | 140.56 | mg | 140.56 | mg | 140.56 | mg | 70.28 | mg |
| Capsule Shell | | | | | | | | |

| | | | | |
|---|---|---|---|---|
| Oil/drug ratio versus | 4:1 | 2:1 | 1:1 | 2:1 |
| reference formulation | | | (reference | |
| | | | formula) | |

^{a}Used in manufacturing process. Not part of drug composition..

Preparation of Gelatin Capsules:
Preparation of 1 mcg Gelatin Capsule—Formulation 1

0.300 g paricalcitol and 4.800 g butylated hydroxytoluene (BHT) were dissolved in 426.0 g dehydrated ethanol, non-denatured. Dissolution was verified by visual inspection. The resulting solution was combined with 42.168 kg Neobee M-5 Oil, and mixed to homogeneity. Potency and homogeneity of the fill solution were verified by HPLC using an external standard. The fill solution was encapsulated to prepare soft gelatin capsules with a fill weight of 142 mg.

Preparation of 2 mcg Gelatin Capsule—Formulation 2

0.600 g paricalcitol and 4.800 g BHT were dissolved in 426.0 g dehydrated ethanol, non-denatured. Dissolution was verified by visual inspection. The resulting solution was combined with 42.168 kg Neobee M-5 Oil, and mixed to homogeneity. Potency and homogeneity of the fill solution were verified by HPLC using an external standard. The fill solution was encapsulated to prepare soft gelatin capsules with a fill weight of 142 mg.

Preparation of 4 mcg Gelatin Capsule—Formulation 3

1.200 g paricalcitol and 4.800 g BHT were dissolved in 426.0 g dehydrated ethanol, non-denatured. Dissolution was verified by visual inspection. The resulting solution was combined with 42.168 kg Neobee M-5 Oil, and mixed to homogeneity. Potency and homogeneity of the fill solution were verified by HPLC using an external standard. The fill solution was encapsulated to prepare soft gelatin capsules with a fill weight of 142 mg.

Preparation of 1 mcg Gelatin Capsule—Formulation 4

0.600 g paricalcitol and 4.800 g BHT were dissolved in 426.0 g dehydrated ethanol, non-denatured. Dissolution was verified by visual inspection. The resulting solution was combined with 42.168 kg Neobee M-5 Oil, and mixed to homogeneity. Potency and homogeneity of the fill solution were verified by HPLC using an external standard. The fill solution was encapsulated to prepare soft gelatin capsules with a fill weight of 71 mg.

Fill solutions for Formulations 1, 2, and 3 were “proportionately similar” per definition 3 of the FDA Guidance (March 2003) set forth above. Fill solutions for Formulations 2 and 4 were “proportionately similar” per definition 1 of the FDA Guidance set forth above. Capsule shell qualitative and quantitative excipient compositions were varied without regard to maintaining compositional proportionality across the formulations.

The fact that Formulation 1 was not bioequivalent to the reference formulation (Formulation 3), despite being proportionally similar to it was unexpected and could not have been known a priori.

It also could not have been known a priori that Formulation 4 would be bioequivalent to the reference formulation (Formulation 3). Formulation 4 is not proportionally similar to Formulation 3 by any definition. The proportional similarity of Formulations 2 and 3 (by definition 3) and Formulations 2 and 4 (by definition 1 of the FDA Guidance (March 2003) did not ensure bioequivalence of Formulations 3 and 4.

In general, bioequivalence between dosage forms cannot assume that if A=B and B=C that A will be bioequivalent to C. In this example, where proportionally similar formulations were not bioequivalent (e.g., Formulations 1 and 3) whether non-proportionally similar Formulations 3 and 4 would be bioequivalent could not be known without experiment.

Methodology:

Once sufficient quantities of each of the above four (4) formulations were prepared, a study was conducted to assess the bioequivalence of Formulations 1-4 (Table 1). This was a Phase 1, single-dose open label, randomized, fasting study conducted with 88 subjects according to a three-cohort, four period, crossover dose strength linking design. Each regimen was administered as a single 8 μg dose.

Subjects were randomly assigned in equal numbers to receive one of four sequences of Regimen A: eight 1 μg paricalcitol capsules; Regimen B: eight 1 μg paricalcitol capsules; Regimen C: four 2 μg paricalcitol capsules; Regimen D: two 4 μg paricalcitol capsules. Each regimen was administered with 240 mL of water after a 10-hour fast and approximately 4 hours prior to lunch. A washout interval of at least 7 days separated the doses in each of the four study periods.

For determination of paricalcitol in plasma, blood samples were collected by venipuncture into 7-mL evacuated collection tubes containing edetic acid (EDTA) prior to dosing (0 hour) and at 0.5, 1, 1.5, 2, 2.5, 3, 3.5, 4, 5, 6, 8, 12, 18, 24, 36 and 48 hours after dosing in each study period. Sufficient blood was collected to provide 2.5 mL plasma from each sample.

Plasma concentrations of paricalcitol were determined using a validated HPLC-tandem mass spectrometric method at Abbott Laboratories, Abbott Park, Ill. The lower limit of quantitation (LLOQ) of paricalcitol was 0.021 ng/mL using a 0.6 mL plasma sample. Plasma concentrations could not be estimated at any sampling time points in 1 period for 5 subjects in 3 periods for 1 subject and in all 4 periods for 2 subjects because the area of a flanking endogenous peak could not be separated from the paricalcitol peak in the LC/MS/MS assay. Additionally, for one subject, all concentration values were below the quantitation limit for Period 2, Regimen D. Data for this subject from Period 2 (Regimen D) were excluded from the statistical analysis of log-transformed pharmacokinetic parameters.

Number of Subjects:

Planned: 88; Entered: 88; Completed: 85; Evaluated for Safety: 88; Evaluated for Pharmacokinetics: 83

For the 88 subjects that participated in the study, the mean age was 37.8 years (ranging from 19 to 54 years), the mean weight was 70.2 kg (ranging from 50 to 96 kg) and the mean height was 164.4 cm (ranging from 146 to 189 cm). For the 83 subjects included in the pharmacokinetic analyses, the mean age was 37.8 years (ranging from 19 to 54 years), the mean weight was 71.0 kg (ranging from 54.4 to 96.0 kg) and the mean height was 164.9 cm (ranging from 146 to 189 cm).

A post-study audit of the clinical site uncovered two subjects who participated in another paricalcitol study concurrent to their participation in this study. This was a protocol violation of one of the exclusion criteria. Therefore, data from these subjects was not included in any of the individual or summary calculations or statistical analyses of the pharmacokinetic parameters.

Diagnosis and Main Criteria for Inclusion: Subjects were male and female volunteers between 18 and 55 years of age, inclusive. Subjects in the study were judged to be in general good health based on the results of his/her medical history, tobacco and alcohol use histories, physical examination (including vital signs), laboratory profile and 12-lead electrocardiogram (ECG). Females were postmenopausal, sterile, or if of childbearing potential, were not pregnant or breast-feeding and were practicing an acceptable method of birth control.

Criteria for Evaluation:

Pharmacokinetic: The pharmacokinetic parameter values of paricalcitol for each of the above four (4) formulations were estimated using noncompartmental methods. These included: the maximum plasma concentration (Cₘₐₓ) and time to Cₘₐₓ (Tₘₐₓ), the terminal phase elimination rate constant (β), terminal phase elimination half-life (t_{1/2}), the area under the plasma concentration-time curve (AUC) from time 0 to time of the last measurable concentration (AUC₀₋ₜ), the AUC from time 0 to infinity (AUC_{0-∞}), apparent oral clearance (CL/F) and apparent volume of distribution (Vd_{β}/F). These results are shown below in Table 2.

Safety: Safety was evaluated based on assessments of adverse events, vital signs, physical examinations, ECG and laboratory tests.

TABLE 2
Pharmacokinetic Results: Mean ± standard deviation (SD)
pharmacokinetic parameters of paricalcitol after administration
of the four formulations described above are listed below.

| | Regimen^{£} | | | |
|---|---|---|---|---|
| | A | B | C | D |
| Pharmacokinetic | 1 μg Capsules | 1 μg Capsules | 2 μg Capsules | 4 μg Capsules |
| Parameters (units) | (N = 82)‡ | (N = 82)‡ | (N = 80) | (N = 80)‡ |
| Cₘₐₓ (ng/mL) | 0.228 ± 0.080 | 0.277 ± 0.116* | 0.262 ± 0.070* | 0.284 ± 0.092* |
| Tₘₐₓ (h) | 3.2 ± 1.4 | 3.0 ± 1.5 | 2.9 ± 1.6 | 2.5 ± 1.1*† |
| AUC₀₋ₜ (ng · h/mL) | 1.95 ± 0.92 | 2.21 ± 0.90* | 2.16 ± 0.95* | 2.28 ± 1.00* |
| AUC_{0-∞} (ng · h/mL) | 2.42 ± 1.34 | 2.57 ± 1.00* | 2.65 ± 1.28* | 2.67 ± 1.11* |
| β^{§} (1/h) | 0.112 ± 0.050 | 0.120 ± 0.050 | 0.118 ± 0.053 | 0.111 ± 0.045 |
| t_{1/2}^{$#} (h) | 6.2 ± 2.8 | 5.8 ± 2.4 | 5.9 ± 2.7 | 6.2 ± 2.6 |
| CL/F^{#} (L/h) | 4.17 ± 2.50 | 3.84 ± 2.67 | 3.68 ± 1.67 | 3.56 ± 1.85 |
| Vd_{β}/F^{#} (L) | 39.16 ± 18.20 | 31.29 ± 10.32 | 33.52 ± 10.99 | 33.00 ± 12.31 |

^{£}Regimen A: Eight 1 μg paricalcitol capsules
Regimen B: Eight 1 μg paricalcitol capsules
Regimen C: Four 2 μg paricalcitol capsules.
Regimen D: Two 4 μg paricalcitol capsules).
All regimens were administered as a single 8 μg dose under fasting conditions.
*Statistically significantly different from Regimen A (ANOVA, p < 0.05).
†Statistically significantly different from Regimen B (ANOVA, p < 0.05).
^{$}Harmonic mean ± pseudo-standard deviation; evaluations of t_{1/2} were based on statistical tests for β.
‡N = 80 for β, t_{1/2,} and Vd_{β}/F for Regimen A. N = 81 for β, t_{1/2,} and Vd_{β}/F for Regimen B and N = 79 for Tₘₐₓ, β, t_{1/2,}, CL/F and Vd_{β}/F for Regimen D.
^{§}β was not estimable for 2 subjects, 1 subject, and 1 subject for Regimens A, B and D, respectively.
^{#}Parameter was not tested statistically.

Statistical Methods:

Pharmacokinetic: An analysis of variance (ANOVA) was performed for Tₘₐₓ, β and the natural logarithms of Cₘₐₓ and AUC. The model included effects for cohort, sequence, cohort by sequence interaction, subject nested within cohort and sequence combination, period, regimen and the interactions of cohort with each of period and regimen. The effect of subject was random while all other effects were fixed. For the tests on cohort, sequence, and cohort by sequence interaction, the denominator sum of squares for the F statistic was the sum of squares for subject nested within the cohort and sequence combination. For the tests on all other effects, the denominator sum of squares was the residual sum of squares. Within the ANOVA modeling framework, the four regimens were compared pairwise with a significance level of 0.05.

The relative bioavailability of each of the six pairs of regimens was assessed by a two one-sided tests procedure via 90% confidence intervals obtained from the analyses of the natural logarithms of Cₘₐₓ and AUC. These confidence intervals were obtained by exponentiating the endpoints of confidence intervals for the difference of mean logarithms obtained within the framework of the ANOVA model for each comparison.

The results are shown below in Table 3.

TABLE 3

| | | | | Relative Bioavailability | |
|---|---|---|---|---|---|
| Regimens | Pharmacokinetic | Central Value* | | Point | 90% Confidence |
| Test vs. Reference | Parameter | Test | Reference | Estimate⁺ | Interval |
| A vs. B | Cₘₐₓ | 0.215 | 0.256 | 0.839 | 0.789-0.891 |
| | AUC₀₋ₜ | 1.787 | 2.020 | 0.885 | 0.821-0.953 |
| | AUC_{0-∞} | 2.209 | 2.397 | 0.922 | 0.862-0.985 |
| A vs. C | Cₘₐₓ | 0.215 | 0.253 | 0.848 | 0.798-0.902 |
| | AUC₀₋ₜ | 1.787 | 1.990 | 0.898 | 0.834-0.968 |
| | AUC_{0-∞} | 2.209 | 2.416 | 0.915 | 0.855-0.978 |
| A vs. D | Cₘₐₓ | 0.215 | 0.271 | 0.793 | 0.745-0.843 |
| | AUC₀₋ₜ | 1.787 | 2.057 | 0.868 | 0.806-0.936 |
| | AUC_{0-∞} | 2.209 | 2.457 | 0.899 | 0.841-0.962 |
| B vs. C | Cₘₐₓ | 0.256 | 0.253 | 1.012 | 0.952-1.075 |
| | AUC₀₋ₜ | 2.020 | 1.990 | 1.015 | 0.942-1.094 |
| | AUC_{0-∞} | 2.397 | 2.416 | 0.992 | 0.928-1.061 |
| B vs. D | Cₘₐₓ | 0.256 | 0.271 | 0.945 | 0.889-1.005 |
| | AUC₀₋ₜ | 2.020 | 2.057 | 0.982 | 0.911-1.059 |
| | AUC_{0-∞} | 2.397 | 2.457 | 0.975 | 0.912-1.044 |
| C vs. D | Cₘₐₓ | 0.253 | 0.271 | 0.934 | 0.878-0.994 |
| | AUC₀₋ₜ | 1.990 | 2.057 | 0.967 | 0.897-1.043 |
| | AUC_{0-∞} | 2.416 | 2.457 | 0.983 | 0.919-1.052 |

*Antilogarithm of the least squares means for logarithms.
⁺Antilogarithm of the difference (test minus reference) of the least squares means for logarithms.

Safety: The number and percentage of subjects reporting treatment-emergent adverse events were tabulated by COSTART V term and body system with a breakdown by regimen. Laboratory test values outside the reference ranges were flagged and evaluated for clinical significance.

Safety Results: Twenty-five (25) of 88 subjects (28.4%) reported at least one treatment-emergent adverse event in any regimen. The most common treatment-emergent adverse events (reported by 4 or more subjects overall) were headache, nausea, and rash. There were no apparent differences among the regimens with respect to safety.

No deaths or serious adverse events occurred during the study. No treatment-emergent adverse events were considered probably related to study drug and most of the treatment-emergent adverse events were considered mild in severity. One subject withdrew consent after completing Regimens A and B. Two subjects discontinued prematurely due to adverse events that were considered probably not related and not related to study drug. No clinically significant changes in laboratory values, vital signs, ECG results or physical examination were identified in the study. No new or unexpected patterns of adverse event occurrences were identified with the administration of 8 μg of paricalcitol as eight 1 μg capsules (Regimen A), as eight 1 μg capsules (Regimen B), as four 2 μg capsules (Regimen C), or as two 4 μg capsules (Regimen D).

Conclusions: The results of this study demonstrate that Regimens B (8×1 μg capsules, formulation 4; oil/drug ratio=2:1), C (4×2 μg capsules, formulation 2; oil/drug ratio=2.1) and D (2×4 μg capsules formulation 3; oil/drug ration=1:1) were bioequivalent because the 90% confidence intervals for evaluating bioequivalence were contained within the 0.80 to 1.25 range. Regimen A (8×1 μg capsules, formulation 1; oil/drug ratio=4.1) was equivalent to Regimens B, C and D with respect to AUC₀₋ₜ and AUC_{0-∞}; however, equivalence could not be concluded for Cₘₐₓ since the lower limit of the 90% confidence intervals extended slightly below 0.80.

The regimens tested were generally well tolerated by the subjects. No apparent differences were seen among the regimens in their adverse event profiles. No clinically significant laboratory values, vital signs values, ECG results or physical examination were observed during the study. There was no apparent trend in any of the safety variables associated with any of the paricalcitol dosing regimens studied. There were no apparent differences among the regimens with respect to safety.

### EXAMPLE 2

### Prophetic Example Describing Certain Oral Paricalcitol Formulations and Methods of Making Said Formulations

The following 0.25, 16.0 and 32.0 mcg oral formulations of paricalcitol shown in Table 4 below can be prepared into capsules (soft or hard) or tablets using routine techniques known in the art.

TABLE 4

| | | | | | Formulation K | | | |
|---|---|---|---|---|---|---|---|---|
| | Formulation I | | Formulation J | | Unit | | Formulation L | |
| Ingredients | Unit Formula | | Unit Formula | | Formula | | Unit Formula | |
| Fill Solution | | | | | | | | |
| Paricalcitol | 1 | mcg | 0.25 | mcg | 16 | mcg | 32 | mcg |
| Dehydrated Alcohol | 0.71 | mg | 0.18 | mg | 11.36 | mg | 22.72 | mg |
| Medium Chain | 70.23 | mg | 17.56-68.47 | mg | 288.12-1123.68 | mg | 576.24-2247.36 | mg |
| Triglyceride Oil | | | | | | | | |

These formulations can be encapsulated in an amount of suitable matrix that provides a pharmaceutically acceptable oral dosage form including, but not limited to, soft gelatin, hard gelatin, hydroxylpropyl ethyl cellulose and polymethacrylates. Optionally, additional excipients can be added to these formulations. Such added excipients can be present in an amount that can be readily determined by one of ordinary skill in the art and are not limited to the non-polar solvent:drug ratio described herein. For the purpose of this example, Formulation I serves as the “selected reference formulation.”

The amount of medium chain triglyceride used in Formulations J, K, and L will be suitable for formulations according to the invention if they fall within the ranges to optimize unit dose manufacture. The resulting formulations will be bioequivalent to the selected reference formulation since the ratio of oil:drug range from compositionally proportionate to different by no more than a factor of about 4.

Preparation of 1 mcg Formulation

The 1 mcg oral formulation can be prepared as described in Example 1 in Formulation 4.

Preparation of 0.25 mcg Formulation

0.150 g paricalcitol can be dissolved in 108.0 g dehydrated ethanol, non-denatured. Dissolution could be verified by visual inspection. The resulting solution can be combined with an amount of Medium Chain Triglycerides within the range of 10.536-41.082 kg and mixed to homogeneity. Potency and homogeneity of the fill solution can be verified by HPLC using an external standard. The fill solution can be used to produce 600,000 units for unit dose administration by methods known in the art.

Preparation of 16 mcg Formulation

4.800 g paricalcitol can be dissolved in 6.816 kg dehydrated ethanol, non-denatured. Dissolution could be verified by visual inspection. The resulting solution can be combined with an amount of Medium Chain Triglycerides within the range of 172.87-674.21 kg and mixed to homogeneity. Potency and homogeneity of the fill solution can be verified by HPLC using an external standard. The fill solution can be used to produce 600,000 units for unit dose administration by methods known in the art.

Preparation of 32 mcg Formulation

9.600 g paricalcitol can be dissolved in 13.632 kg dehydrated ethanol, non-denatured. Dissolution could be verified by visual inspection. The resulting solution can be combined with an amount of Medium Chain Triglycerides within the range of 345.74-1,348.4 kg and mixed to homogeneity. Potency and homogeneity of the fill solution can be verified by HPLC using an external standard. The fill solution can be used to produce 600,000 units for unit dose administration by methods known in the art.

### EXAMPLE 3

### Safety and Bioavailability of Oral Formulations of Paricalcitol in Subjects with End-Stage Renal Disease Undergoing Hemodialysis Treatment

In this example, a study was conducted to assess the safety and bioavailability of a paricalcitol capsule formulation relative to that of a paricalcitol intravenous formulation in subjects with end-stage renal disease undergoing hemodialysis treatment.

Methodology: This was a Phase I, open-label, randomized, single-dose, two-period, crossover, nonfasting study. Subjects were randomized into two sequence groups of Regimens A and B. The two nonfasting study regimens were:

Regimen A: Paricalcitol capsule formulation (0.24 μg/kg) administered orally with 180 mL of water in strengths of 0.5, 1, 2 or 4 μg.

Regimen B: Paricalcitol intravenous formulation (0.24 μg/kg) administered as an intravenous bolus injection of 5 μg/mL. The intravenous formulation contained 2-10 micrograms/milliliter of paricalcitol, 30% (v/v) propylene glycol, 20% (v/v) ethanol and 50% (v/v) water.

Both regimens were administered at the end of a regular hemodialysis session on Study Day 1, 30 minutes after a breakfast was served. Phosphate binders, commonly used in the management of end-stage renal disease, were withheld 8 hours prior to and for 2 hours after the drug administration. Fourteen subjects participated in the study. Twelve subjects completed both regimens of the study. A washout interval of at least 7 days separated the doses of the two study periods. For the paricalcitol intravenous formulation, the blood samples (7 mL) were collected into evacuated collection tubes containing EDTA from the arm contralateral to the injection arm immediately prior to dosing (0 hours), at 5 and 30 minutes, and at 1, 2, 3, 4, 6, 8, 12, 24, and 48 hours post-dose. For the paricalcitol capsule formulation, the blood samples (7 mL) were collected into evacuated collection tubes containing EDTA immediately prior to dosing (0 hours), at 30 minutes, and at 1, 1.5, 2, 3, 4, 6, 8, 12, 24 and 48 hours post-dose. Plasma concentrations of paricalcitol were determined using a validated liquid chromatography—tandem mass spectrometric assay method at Abbott Laboratories, Abbott Park, Ill. (a proprietary method of Abbott Laboratories). The lower limit of quantification of paricalcitol was 0.02 ng/mL using a 0.6 mL plasma sample.

Number of Subjects:

Planned: 12 Entered: 14 Completed: 12 Evaluated for Safety: 14

Evaluated for Pharmacokinetics: 14 (Regimen A) and 12 (Regimen B)

Diagnosis and Main Criteria for Inclusion: Subjects (14) were male and female volunteers between 18 and 75 years of age, inclusive. Subjects had end-stage renal disease, and had undergone maintenance hemodialysis at least 2 months prior to entry into the study. In addition, subjects were on maintenance hemodialysis three times a week, and were expected to remain on hemodialysis during the course of the study. Female subjects of childbearing potential were not pregnant or breast-feeding and were practicing an acceptable method of birth control. Normalized serum calcium (Ca⁺⁺) level was ≦10.5 mg/dL and a calcium—phosphorus (Ca×P) product was ≦70.

Reference Therapy, Dose/Strength/Concentration and Mode of Administration: The oral dosing in Regimen A was accomplished with a combination of 0.5, 1, 2 and 4 μg capsule strengths. The intravenous dosing in Regimen B was accomplished with a 5 g/mL intravenous formulation.

Duration of Treatment: Each subject was given a single dose with 2½ days of confinement in each of two periods.

Criteria for Evaluation:

Pharmacokinetics: The pharmacokinetic parameter values of paricalcitol were estimated using noncompartmental methods. These included: the maximum observed concentration (Cₘₐₓ), the elimination rate constant (β), half-life (t_{1/2}), the area under the plasma concentration-time curve from time 0 to time of the last measurable concentration (t) (AUC₀₋ₜ), the AUC from time 0 to infinity (∞) (AUC_{0-∞}) and the clearance ([CL] for intravenous administration and apparent total oral clearance [CL/F] for oral administration). The time to Cₘₐₓ(Tₘₐₓ) was estimated after oral administration only. The volume of distribution for intravenous administration (V_{dβ) and apparent volume of distribution for oral administration (V}_{dβ}/F) value were calculated by dividing the clearance by β.

Safety: Safety was evaluated based on vital signs, physical examinations, laboratory tests, electrocardiogram (ECG) and adverse events assessments throughout the study.

Statistical Methods: An analysis of variance (ANOVA) was performed for β and the logarithms of Cₘₐₓ, AUC₀₋ₜ, and AUC_{0-∞}. Within the framework of the ANOVA for the logarithms of Cₘₐₓ, AUC₀₋ₜ, and AUC_{0-∞}, a 95% confidence interval for the bioavailability of the capsule formulation relative to that of the intravenous formulation was obtained.

The number and percentage of subjects reporting adverse events were tabulated by COSTART term and body system. Laboratory values outside the reference ranges were flagged in the data listings, and evaluated for clinical significance.

Conclusions:

Pharmacokinetic Results: Mean±standard deviation (SD) pharmacokinetic parameters of paricalcitol are listed in the following Table 5.

TABLE 5

Regimen

| Pharmacokinetic | A (Oral) | B (Intravenous) |
|---|---|---|
| Parameters | (N = 14) | (N = 12) |
| Cₘₐₓ (ng/mL) | 0.575 ± 0.172* | 1.680 ± 0.511 |
| Tₘₐₓ (h) | 4.0 ± 3.3 | ND |
| AUC₀₋ₜ (ng · h/mL) | 10.22 ± 2.63* | 12.69 ± 3.24 |
| AUC_{0-∞} (ng · h/mL) | 11.67 ± 3.23* | 14.51 ± 4.12 |
| β (1/h) | 0.050 ± 0.017 | 0.050 ± 0.023 |
| t_{1/2} (h)$ | 13.9 ± 5.1 | 13.9 ± 7.3 |
| CL (L/h)†φ | 1.82 ± 0.75 | 1.49 ± 0.60 |
| V_{dβ} (L)†φ | 38.0 ± 16.4 | 30.8 ± 7.5 |

*Statistically significantly different from Regimen B (p < 0.05).
$Harmonic mean ± pseudo-standard deviation; evaluations of t_{1/2} were based on statistical tests for β.
†Parameter was not tested statistically.
φCL for Regimen B and CL/F for Regimen A; V_{dβ} for Regimen B and V_{dβ}/F for Regimen A.
ND: Not Determined.

The absolute bioavailability results for paricalcitol are listed in the following Table 6.

TABLE 6

| | | Central Values* | | Absolute Bioavailability | |
|---|---|---|---|---|---|
| Regimens | Pharmacokinetic | Oral | IV | Point | 95% Confidence |
| Test vs. Reference | Parameter | Regimen A | Regimen B | Estimate⁺ | Interval |
| A vs. B | Cₘₐₓ | 0.545 | 1.686 | 0.324 | 0.246-0.426 |
| | AUC₀₋ₜ | 9.878 | 12.723 | 0.776 | 0.671-0.898 |
| | AUC_{0-∞} | 11.213 | 14.230 | 0.788 | 0.669-0.929 |

*Antilogarithm of the least squares means for logarithms.
⁺Antilogarithm of the difference (test minus reference) of the least squares means for logarithms.
IV Intravenous

Safety Results: A total of 93% (13/14) subjects reported at least one treatment-emergent adverse event, in either regimen (50%, 7/14 in Regimen A and 100%, 12/12 in Regimen B). The most frequently reported adverse event was application site reaction (83%, 10/12), associated with the intravenous administration of paricalcitol injection (Regimen B). Overall, 36% of the adverse events were considered probably related, and 3.4% possibly related to the paricalcitol. The remaining adverse events were probably not, or not related to the paricalcitol. In Regimen A, two subjects reported mild and five subjects reported moderate adverse events. No severe adverse events were reported with this regimen. Adverse events considered possibly or probably related to the paricalcitol in Regimen A were pain, nausea, phlebitis, and taste perversion, all reported by the same subject. In Regimen B, six subjects reported mild, four subjects reported moderate, and two subjects severe adverse events. The two subjects with severe adverse events experienced pain with intravenous injection of paricalcitol. The pain was alleviated by flushing the injection tubing with saline. Adverse events considered possibly or probably related to the paricalcitol in Regimen B were injection site pain, injection site reaction, pain, thrombophlebitis, vascular disorder, edema, dizziness, application site reaction, and taste perversion.

One subject was prematurely discontinued from the study due to an adverse event not related to the paricalcitol. No deaths were reported during the study. Three treatment-emergent serious adverse events were reported by three subjects during the study. Two were considered not related and one was considered probably not related to the paricalcitol. No clinically significant changes in vital signs, physical examination results, or ECG measurements were observed during the course of the study. No trends of changes in laboratory variables were observed for any regimen during the study.

Conclusions: The estimated absolute bioavailability of paricalcitol administered as a single oral dose under nonfasting conditions in subjects with end-stage renal disease who were undergoing hemodialysis was 78.8%. The harmonic mean t_{1/2} of paricalcitol in subjects with end-stage renal disease who were undergoing hemodialysis was approximately 14 hours.

The regimens administered in this study were generally safe and well tolerated. No new or unexpected patterns of adverse event occurrences were identified with the administration of paricalcitol capsule or intravenous injection. Except for application site reaction observed during the administration of the paricalcitol injection in Regimen B, no apparent differences between the paricalcitol capsule and injection with respect to safety were identified.

### EXAMPLE 4

### Safety, Pharmacokinetics and Pharmacodynamics of Single and Multiple Doses of Oral Paricalcitol formulations

In this example, a study was conducted to evaluate the safety, pharmacokinetics and pharmacodynamics of single and multiple doses of oral paricalcitol formulations in subjects with moderate to severe chronic renal impairment. As used herein, “subjects with moderate to severe chronic renal impairment” means that said subjects suffer from CKD Stage 3 and Stage 4.

Methodology: This was a Phase 1, open-label, single and multiple-dose, multi-center study. A sufficient number of subjects were screened to have approximately 15 subjects with moderate renal impairment (CKD Stage 3) (Group 1, Glomerular Filtration Rate (GFR) of 30-60 mL/min) and approximately 15 subjects with severe renal impairment (CKD Stage 4) (Group 2, GFR <30 mL/min, not requiring dialysis) enrolled in the study. The subjects in moderate renal impairment group received 4 μg paricalcitol capsule on Study Day 1, and 4 μg QD for 6 doses over Study Days 3-8. The subjects in severe renal impairment group received 3 μg paricalcitol capsule on Study Day 1 and 3 μg QD for 6 doses over Study Days 3-8. Each dose of study was taken orally with approximately 180 mL of water at 0800, 30 minutes after breakfast was served.

Blood samples for paricalcitol assay were collected by venipuncture into 7 mL evacuated EDTA-containing collection tubes within 5 minutes prior to dosing (0 hour) and at 0.5, 1, 2, 3, 4, 6, 8, 12, 24, 36, and 48 hours after dosing on Study Days 1 and 8. Sufficient blood was collected to provide approximately 2.5 mL plasma from each sample. In addition, urine was collected for 48 hours following Study Day 1 dosing. Serum and urine pharmacodynamic markers, were measured at selected time points in the study. Also, a 24 hr urine amount of urine pharmacodynamic markers were measured following paricalcitol dose on Study Days 1 and 8.

Plasma concentrations of paricalcitol were determined using a validated liquid chromatography method with tandem mass spectrometric assay method at Abbott Laboratories, Ill. The lower limit of quantitation (LLOQ) for paricalcitol was established at 0.01022 ng/mL using a 0.6 mL plasma sample.

Urinary concentrations of paricalcitol were determined using a validated HPLC method with tandem mass spectrometric assay method at Abbott GmbH & Co. KG, Ludwigshafen, Germany. The LLOQ for paricalcitol was established at 0.05 ng/mL using a 0.9 mL urine sample.

Number of Subjects (Planned and Analyzed):

Planned: 30; Entered: 29; Completed: 28; Evaluated for Safety: 29; Evaluated for

Pharmacokinetics: 28

For the 29 subjects who participated in the study, the mean age was 62.0 years (ranging from 39 to 76 years), the mean weight was 83.4 kg (ranging from 52 to 112 kg) and the mean height was 170.5 cm (ranging from 153 to 189 cm). For the 15 subjects included in the pharmacokinetic analyses in moderate renal impairment group, the mean age was 63.9 years (ranging from 49 to 76 years), the mean weight was 83.4 kg (ranging from 52 to 112 kg) and the mean height was 168.7 cm (ranging from 153 to 182 cm). For the 14 subjects included in the pharmacokinetic analyses in severe renal impairment group, the mean age was 59.9 years (ranging from 39 to 76 years), the mean weight was 83.3 kg (ranging from 57 to 103 kg) and the mean height was 172.5 cm (ranging from 155 to 189 cm).

Diagnosis and Main Criteria for Inclusion: Subjects were male and female subjects between 18 and 75 years, inclusive. Approximately half of the subjects in the study were judged to have moderate renal impairment (CKD Stage 3) (Group 1, GFR of 30-60 mL/min) and the other half with severe renal impairment (CKD Stage 4) (Group 2, GFR<30 mL/min, not requiring dialysis). Females were postmenopausal, sterile or were not pregnant or breast-feeding and were practicing at least one of the acceptable methods of birth control specified in the protocol.

Test Product Therapy, Dose/Strength/Potency and Mode of Administration:

Paricalcitol Capsule Dose Strength

TABLE 7

Paricalcitol Capsule
Dose Strength

| Dosage Form | Soft Capsule | Soft Capsule |
|---|---|---|
| Formulation | Oral | Oral |
| Strength | 1 ug | 4 ug |
| Potency (% of Label Claim) | 97% | 100% |
| Batch Size | 400,000 | 200,000 |

Fill Formulation Information for Soft Capsules Tested

| Ingredients | Capsule | Capsule |
|---|---|---|
| Fill Solution | | |
| Paricalcitol | 4 mcg | 1 mcg |
| Dehydrated Alcohol | 1.42 mg | 0.71 mg |
| BHT | 16 mcg | 8 mcg |
| Neobee M-5 Oil | 140.56 mg | 70.28 mg |

These capsules can be made as described in Example 1 for Formulations 4 and 3.

Duration of Treatment: Paricalcitol was administered on Study Day 1 and from Study Day 3 through Study Day 8.

Criteria for Evaluation:

Pharmacokinetic: Values for the pharmacokinetic parameters of paricalcitol, including the maximum observed plasma concentration (Cₘₐₓ), the time to Cₘₐₓ(Tₘₐₓ), the terminal phase elimination rate constant (β), half life (t_{1/2}), area under the plasma concentration-time curve (AUC), apparent total clearance (CL/F), and apparent volume of distribution (Vdβ/F) were determined using noncompartmental methods. Values of these parameters were determined after both single (first) dose (Study Day 1) and multiple doses (Study Day 8). In addition, minimum observed plasma concentration (Cmin), accumulation index (AI), and degree of fluctuation (DFL) after multiple doses were determined for the dose on Study Day 8 (steady state).

Pharmacodynamic: Serum pharmacodynamic markers [serum calcium, calcium-phosphorus product (Ca×P), serum phosphorus, serum bone specific alkaline phosphatase (AP), serum osteocalcin, serum C-terminal telopeptide of collagen (CTx), serum tartarate resistant acid phosphatase-type 5b (TRAP-5b), intact (iPTH) and whole PTH] were measured from samples collected immediately prior to dosing on Study Days 1, 3, 5, and 8. One additional sample was collected 48 hours following the Study Day 8 dose (on the morning of Study Day 10). Urine pharmacodynamic markers [calcium, creatinine and deoxy-pyridinoline (DPD)] were measured from samples collected immediately prior to dosing on Study Days 1 and 8, and during the intervals 0 to 4, 4 to 8, 8 to 12, and 12 to 24 hours after dosing on Study Days 1 and 8. In addition, these markers were measured using the first morning void sample on Study Days 3, 5, and 10.

Safety: Safety was evaluated based on assessments of adverse event monitoring and vital signs, physical examinations, ECGs and laboratory tests assessments.

Statistical Methods:

Pharmacokinetic: Point estimates and the corresponding 95% confidence intervals were obtained for central values of the pharmacokinetic parameters for each of the renal impairment groups. Two-sample t-test was performed to compare the pharmacokinetic parameters across the two renal impairment groups on each of Study Days 1 and 8. Repeated Measurement Analysis was performed for each group on the plasma total paricalcitol concentration trough values on Study Days 6, 7, 8, and 24 h post Study Day 8 dose to examine whether or not steady state was achieved at Study Day 8. Within the framework of the analysis, the mean trough concentration values on Study Days 6, 7 and 8 were each compared to that at 24 h post Study Day 8 dose.

An analysis of variance (ANOVA) was performed on Tₘₐₓ, β, and the logarithms of AUC and Cₘₐₓ to compare the single-dose and steady-state data for each of the renal impairment groups. The same test was also performed for each group for the difference between the natural logarithm of the AUC₀₋₂₄ on Day 8 and the natural logarithm of the AUC_{0-∞} on Study Day 1. Then the point estimate and 95% confidence interval for the ratio of the central values (the central value of the AUC₀₋₂₄ on Study Day 8 to that of the AUC_{0-∞} on Study Day 1) were obtained by exponentiating the corresponding point estimate for the difference.

Pharmacodynamic: Two-sample t-tests were performed to compare baseline pharmacodynamic marker values between the two renal impairment groups. For improving the normality of the distribution of the baseline values, natural logarithm transformations were made to all the pharmacodynamic markers except for the urine calcium and urine creatinine. The relationship between the % change from Study Day 1 to Study Day 10 and the Study Day 1 paricalcitol AUC_{0-∞} was investigated using linear regression. The Study Day 1 AUC_{0-∞} and the corresponding baseline value were independent variables. A one-sample t-test was performed for each group on the change from Study Day 1 to Study Day 8 in 24-hour amount of urine calcium and creatinine.

Safety: The number and percentage of subjects reporting treatment-emergent adverse events were tabulated by COSTART V term and body system for each renal impairment group.

Conclusions:

Pharmacokinetic Results: Non-compartmental mean±SD pharmacokinetic parameters of paricalcitol are listed in the following Table 8.

TABLE 8

| Pharmacokinetic | Moderate Renal Impairment | | Severe Renal Impairment | |
|---|---|---|---|---|
| Parameters (units) | Study Day 1 | Study Day 8 | Study Day 1 | Study Day 8 |
| N | 15 | 15 | 14* | 13 |
| f_{u (%)} | 0.08 ± 0.05 | | 0.08 ± 0.02 | |
| Cₘₐₓ (ng/mL) | 0.113 ± 0.036 | 0.155 ± 0.057^{¥} | 0.065 ± 0.012^{‡} | 0.097 ± 0.023^{¥} |
| Cₘₐₓ/Dose (ng/mL/μg) | 0.028 ± 0.009 | 0.039 ± 0.014 | 0.022 ± 0.004 | 0.032 ± 0.008 |
| Cₘᵢₙ (ng/mL) | — | 0.047 ± 0.018 | — | 0.049 ± 0.010 |
| Tₘₐₓ (h) | 4.7 ± 2.5 | 4.2 ± 1.9 | 5.9 ± 3.6 | 9.1 ± 5.5 |
| AUC₀₋₂₄ (ng · h/mL) | 1.454 ± 0.407 | 2.220 ± 0.701^{§} | 1.020 ± 0.230 | 1.754 ± 0.421^{§} |
| AUC_{0-∞} (ng · h/mL) | 2.424 ± 0.614 | — | 2.127 ± 0.733 | — |
| β (h−¹) | 0.041 ± 0.007 | 0.045 ± 0.009 | 0.035 ± 0.013 | 0.030 ± 0.007^{‡} |
| t_{1/2}^{$} (h) | 16.7612.65 | 15.5313.21 | 19.7017.19 | 22.95 ± 5.63 |
| CL/F† (L/h) | 1.766 ± 0.505 | 2.014 ± 0.774 | 1.517 ± 0.359 | 1.751 ± 0.388 |
| Vd_{β}/F (L) | 43.72 ± 14.45 | 46.76 ± 19.35 | 46.40 ± 12.45 | 61.61 ± 20.34^{¥} |
| AI^{£} | — | 1.54 ± 0.31 | — | 1.7510.39 |
| DFL^{£} (%) | — | 116.77 ± 35.24 | — | 64.86 ± 17.79 |

*N = 14 for Cₘₐₓ and Tₘₐₓ only. N = 13 for rest of the pharmacokinetic parameters
on Study Day 1 in severe renal impairment group.
^{$}Harmonic mean ± pseudo-standard deviation; evaluations of t_{1/2} were based on statistical
tests for R.
†No statistical evaluations performed to compare Study Day 1 CL/F and Study Day 8
CL/F only.
^{£}No statistical evaluations performed.
^{‡}Statistically significantly different between groups on the corresponding Study Day.
^{¥}Statistically significantly different from Study Day 1 within group.
^{§}Statistically significantly different from Study Day 1 AUC_{0-∞} within group.
Mean ± SD pre-dose trough concentrations (ng/mL) on Study Days 6, 7, 8, and 24 h post
dose on Study
Day 8 (Study Day 9) are presented below

| Study Day | Moderate Renal Impairment | Severe Renal Impairment |
|---|---|---|
| 6 | 0.050 ± 0.016 | 0.051 ± 0.011 |
| 7 | 0.053 ± 0.014 | 0.050 ± 0.015 |
| 8 | 0.053 ± 0.019 | 0.056 ± 0.012 |
| 9 | 0.057 ± 0.019 | 0.057 ± 0.014 |

| AUC_{0-∞} on Study Day 1 vs. | | |
|---|---|---|
| AUC₀₋₂₄ on Study Day 8 | Point Estimate⁺ | 95% Confidence Interval |
| Moderate Renal Impairment | 0.8981 | 0.8150-0.9896 |
| Severe Renal Impairment | 0.8380 | 0.7612-0.9227 |

⁺Antilogarithm of the difference (AUC₀₋₂₄ on Study Day 8 vs. AUC_{0-∞} on
Study Day 1) of the least squares means for logarithms.

Pharmacodynamic Results:

At baseline (pre-dose on Study Day 1), the iPTH, whole PTH, serum inorganic phosphorus and osteocalcin concentrations were significantly higher in the severe renal impairment group compared to the moderate renal impairment group (p≦0.0266). However, at baseline, the concentrations of calcium, Ca×P, AP, CTx, and TRAP-5b were not statistically significantly different between the two groups. In subjects with moderate renal impairment, compared to the mean Study Day 1 value, the mean Study Day 10 value for iPTH was statistically significantly lower (p≦0.05), the mean Study Day 10 values for serum calcium, phosphorus, Ca×P and osteocalcin were statistically significantly higher (p<0.0330), and the mean Study Day 10 values of CTx, AP and TRAP-5b were not statistically significantly different.

For whole PTH, a non-parametric test (sign test) was employed due to the skewness of the data. The whole PTH levels decreased significantly on Study Day 10 as compared to that on Study Day 1 (p=0.0413).

In subjects with severe renal impairment, compared to the mean Study Day 1 value, the mean Study Day 10 value for intact and whole PTH was statistically significantly lower (p≦0.05), the mean Study Day 10 values for serum calcium, phosphorus, Ca×P, AP and osteocalcin were statistically significantly higher (p<0.05), and the mean Study Day 10 values of CTx and TRAP-5b were not statistically significantly different.

At baseline, all urine pharmacodynamic marker concentrations were not statistically significantly different between the severe renal impairment group and the moderate renal impairment group. Table 9 below provides the summary of total amount of urine markers excreted over 24 hours after paricalcitol dose on Study Day 1 and Study Day 8 I moderate and severe renal impairment group.

TABLE 9
Total Amount of Urine Markers Excreted Over 24 hours After Dose

| | Moderate Renal | | Severe Renal | |
|---|---|---|---|---|
| | Impairment | | Impairment | |
| Parameter (Unit) | Study Day 1 | Study Day 8 | Study Day 1 | Study Day 8 |
| Creatinine (mg) | 1326.7 ± 566.1 | 1324.4 ± 402.0 | 1088.3 ± 278.6 | 946.7 ± 241.5 [symbol] |
| Calcium (mg) | 49.39 ± 56.85 | 79.24 ± 63.27 | 29.18 ± 20.97 | 50.92 ± 41.18 |
| DPD (nmol) | 52.03 ± 18.96 | 58.60 ± 16.45 | 48.34 ± 21.04 | 47.74 ± 20.47 |
| Ca/Creatinine | 0.045 ± 0.051 | 0.074 ± 0.078 | 0.028 ± 0.019 | 0.054 ± 0.043 [symbol] |

[symbol]

Statistically significantly different from Study Day 1 within group.

Pharmacokinetic-Pharmacodynamic Analysis:

The PK-PD analyses were performed only on PTH, calcium, Ca×P and AP. The effect of AUC_{0-∞} as a covariate on the % change in these markers was tested from Study Day 1 to Study Day 10. Study Day 1 AUC_{0-∞} was not a predictor of the percent (%) change in the above mentioned PD markers except for whole PTH (p=0.0274) in moderate renal impairment group. However, one subject had an unexpectedly large increase in whole PTH. After excluding this subject, the AUC_{0-∞} was no longer a predictor of % change in whole PTH values.

Safety Results: The regimens administered in the study were generally safe and well tolerated. No new or unexpected patterns of adverse event occurrences were identified with the administration of either of the two dose regimens. No apparent differences were identified between the severe renal impairment group and the moderate renal impairment group with respect to safety. A total of 45% (13/29) subjects reported at least one treatment-emergent adverse event. The most frequently reported adverse events were diarrhea, dizziness, and pharyngitis (7% each, 2/29). Overall, 10% of the adverse events were considered probably related or possibly related to study drug. The remaining adverse events were considered by the Investigators to be probably not, or not related to study drug. In the moderate renal impairment group, one subject reported mild adverse events and three subjects reported moderate adverse events. No severe adverse events were reported in this group. Adverse events considered possibly or probably related to study drug in the moderate renal impairment group were rash and pruritus. These events were reported by the same subject.

In the severe renal impairment group, four subjects reported mild adverse events and five subjects reported moderate adverse events. No severe adverse events were reported in this group. Adverse events considered possibly or probably related to study drug in the severe renal impairment group were vomiting and dizziness. Both events were reported by the same subject. One (1) subject was prematurely discontinued from the study due to an adverse event of worsening of an upper respiratory tract infection. This event was considered by the Investigator to be not related to study drug, with an alternative etiology of viral upper respiratory infection.

No deaths or serious adverse events were reported during the study.

No clinically significant trends or changes in vital signs, physical examination results, or ECG measurements were observed during the course of the study.

An increasing trend in serum calcium, phosphorus, and Ca×P levels was observed in both treatment groups, commiserate to the design of this pharmacokinetic study. The study design required supra-pharmacologic, fixed daily doses of oral paricalcitol to allow for study drug assay sensitivity. Unlike the pivotal Phase 3 clinical trials and current clinical practice, subjects were not begun on an initial dose of study drug based on weight or disease severity, with subsequent dose titration to physiological endpoints. The fact that patients with moderate and severe renal impairment have a diminished capacity to excrete calcium and phosphorus, coupled with the supra-pharmacologic, fixed doses employed in this pharmacokinetic study, led to over-suppression of PTH, with resultant increases in serum calcium and phosphorus levels. Additionally, low PTH levels lead to decreased urinary phosphorus excretion with resultant additive effects on increasing serum phosphorus levels.

In summary, in moderate and severe renal impairment groups, there was a significant decrease in PTH levels and increases in calcium, phosphorus, Ca×P levels in response to supra-pharmacologic, fixed doses of paricalcitol used to support paricalcitol assay sensitivity in a 1-week pharmacokinetic study. Results from our 24-week, pivotal Phase 3 studies in CKD Stage 3 and 4 subjects with 2° HPT have shown >90% efficacy in PTH suppression, with no statistically significant differences in episodes of clinically meaningful hypercalcemia, hyperphosphatemia or elevated Ca×P relative to placebo. In these pivotal trials, initial dosing is individualized based on disease severity, with subsequent dose adjustment in response to key physiologic endpoints, consistent with current clinical practice for this disease entity and intended usage for paricalcitol. No new or unexpected patterns of adverse event occurrences were identified with the administration of either of the two doses. No apparent differences were identified between the severe renal impairment group and the moderate renal impairment group with respect to safety.

Conclusions: The pharmacokinetics of paricalcitol in subjects with moderate (CKD Stage 3) and severe (CKD Stage 4) renal impairment were similar to those in subjects with ESRD (CKD Stage 5). The mean half-life of paricalcitol in moderate and severe renal impairment subjects was approximately 16-23 hours, similar to that observed in CKD Stage 5 subjects. Paricalcitol steady state was essentially reached by Study Day 6 in both moderate and severe renal impairment subjects. Paricalcitol pharmacokinetics in moderate and severe renal impairment subjects were essentially time linear. In moderate and severe renal impairment groups, there was a significant decrease in PTH levels and increases in calcium, phosphorus, Ca×P product in response to supra-pharmacologic, fixed doses of paricalcitol used to support paricalcitol assay sensitivity in a 1-week pharmacokinetic study. Results from our 24-week, pivotal Phase 3 studies in CKD Stage 3 and 4 subjects with 2° HPT have shown >90% efficacy in PTH suppression, with no statistically significant differences in episodes of clinically meaningful hypercalcemia, hyperphosphatemia or elevated Ca×P relative to placebo. In these pivotal trials, initial dosing is individualized based on disease severity, with subsequent dose adjustment in response to key physiologic endpoints, consistent with current clinical practice for this disease entity and intended usage for paricalcitol. It can be concluded that the AUC_{0-∞} of paricalcitol is not a significant or useful predictor of responses, when analyzed by a naïve-pool linear regression. In other words, responses to a given exposure vary greatly across individuals, which no doubt is the foundation of current clinical practice of dose individualization through titration and monitoring of PTH, calcium and phosphorus. No significant changes over time were observed with pre-dose concentrations of urinary calcium, creatinine and DPD.

Repeated Paricalcitol dosing does not affect 24-hour urinary excretion of calcium. The regimens administered in the study were generally safe and well tolerated. No new or unexpected patterns of adverse event occurrences were identified with the administration of either of the two dose regimens. No apparent differences were identified between the severe renal impairment group and the moderate renal impairment group with respect to safety.

### EXAMPLE 5

### Single and Multiple Dose Safety and Pharmacokinetic Study of Paricalcitol Oral Formulation Following Daily and Three-Times-a-Week Dosing 1N Subjects in General Good Health

In this example, a study was conducted to assess the single- and multiple-dose safety and pharmacokinetics of an oral paricalcitol formulation following 4 μg daily (QD) and 8 μg three-times-a-week (TIW) administration.

Methodology: This Phase I, multiple-dose, open-label, randomized study was conducted according to an o-period, crossover design. Subjects were randomized into two sequence groups of equal size of Regimens A and B. Subjects received the following regimens:
Regimen A: One 4 μg paricalcitol soft elastic capsule on Study Day 1 and one 4 μg paricalcitol capsule QD for 11 doses on Study Days 3 through 13.
Regimen B: Two 4 μg paricalcitol soft elastic capsules three times a week (8 μg TIW) for 6 doses on Study Days 1, 3, 5, 8, 10, and 12.

The paricalcitol dose was administered at approximately 0700, 30 minutes after breakfast was served on the dosing days of each period. Each dose was administered orally with 240 mL of water. A washout interval of at least 7 days separated the dose of period 1 and dose of period 2.

Seven (7) mL blood samples (to yield at least 3 mL of plasma) were obtained by venipuncture for paricalcitol plasma concentration into appropriately labeled EDTA-containing collection tubes. The blood sampling was as shown below in Table 10.

TABLE 10

| Regimen | Study Day | Sampling Schedule (hour) |
|---|---|---|
| Regimen A (QD) | 1 | 0, 1, 2, 3, 4, 5, 8, 12, 24, and 48 |
| Regimen A (QD) | 12 | 0, 1, 2, 3, 4, 5, 8, and 12 |
| | (second-last dose) | |
| Regimen A (QD) | 13 (last dose) | 0, 1, 2, 3, 4, 5, 8, 12, and 24 |
| Regimen B (TIW) | 1 | 0, 1, 2, 3, 4, 5, 8, 12, 24, and 48 |
| Regimen B (TIW) | 12 (last dose) | 0, 1, 2, 3, 4, 5, 8, 12, 24, and 48 |

0-hour sample = predose sample.

Blood Collection for Paricalcitol Trough Levels: For Regimen A, a blood sample was collected immediately prior to dosing on Study Days 7 and 10. For Regimen B, a blood sample was collected 48 hours after the Study Day 5 dose (Study Day 7) and immediately prior to dosing (0 hour) on Study Day 10.

Plasma concentrations of paricalcitol were determined using a validated liquid chromatography-tandem mass spectrometric assay method at Abbott Laboratories, Abbott Park, Ill. (a proprietary method of Abbott Laboratories). The lower limit of quantification (abbreviated as “LOQ”) of paricalcitol was 0.02 ng/mL using a 0.6 mL plasma sample.

Number of Subjects:

Planned: 20; Entered: 20; Completed: 18; Evaluated for Safety: 20; Evaluated for Pharmacokinetics of Regimens A and B: 19; Evaluated for PK comparing Regimen A to B: 18.

Diagnosis and Main Criteria for Inclusion: Subjects were male and female volunteers between 18 and 55 years of age, inclusive. Subjects in the study were judged to be in general good health based on the results of a medical history, physical examination, laboratory profile and electrocardiogram (ECG). Females were postmenopausal, sterile or if of childbearing potential, were not nursing and were practicing an acceptable method of birth control.

Duration of Treatment: This study was a multiple-dose study with approximately 14 days of confinement in each of the two periods.

Criteria for Evaluation:

Pharmacokinetics: The following pharmacokinetic parameter values were determined using standard non-compartmental methods: maximum observed plasma concentration (Cₘₐₓ), time to Cₘₐₓ(Tₘₐₓ), terminal phase elimination rate constant (β), terminal elimination half-life (t_{1/2}), area under the plasma concentration vs. time curve extrapolated to infinite time (AUC_{0-∞}) following the first dose and area under the plasma concentration vs. time curve over the dose interval at steady state (AUC_{0-τ}; also referred to as AUC₀₋₄₈ or AUC₀₋₂₄ as appropriate). Accumulation index (AI) and degree of fluctuation (DFL) were also evaluated at steady state.

Safety: Safety was evaluated based on adverse event, physical examination, vital signs and laboratory tests assessments.

Statistical Methods: An analysis of variance (ANOVA) was performed to compare single- and multiple-dose pharmacokinetics of paricalcitol. The model included effects for sequence, subject nested within sequence, day (i.e., first dose or last dose), and sequence by day interaction. The effect for subject was random, while all other effects were fixed. The analyzed variables include Tₘₐₓ, β, and the logarithms of AUC and Cₘₐₓ. The AUC value for the single dose was AUC_{0-∞}, while the AUC for the multiple dose was AUC₀₋₂₄. The trough concentrations were analyzed using the same ANOVA model described above to address the issue of steady state attainment for the QD regimen.

At steady state, Regimen A and Regimen B were compared with respect to AUC, Tₘₐₓ, and DFL based on 48-hour measurement following the Study Day 12 dose using a crossover ANOVA model. The logarithmic transformation was also used for AUC. The model had effects for sequence, subject nested within sequence, period and regimen. The effect for subject was random, while all other effects were fixed. For AUC, the two one-sided tests procedure was performed at significance level of 0.05 via a 90% confidence interval for the ratio of central values. Study Day 1 Tₘₐₓ, β, dose-normalized Cₘₐₓ and AUC_{0-∞} from Regimen A and Regimen B were analyzed using the same ANOVA model. In addition, Regimen A Study Day 12 and 13 dose-normalized Cₘₐₓ and AUC₀₋₂₄ were also each compared with Regimen B dose-normalized Cₘₐₓ and AUC₀₋₄₈ using the same model. This same model again was used to compare Regimen A and Regimen B with respect to AI values.

The number and percentage of subjects reporting treatment-emergent adverse events were tabulated by COSTART term and body system with a breakdown by regimen. Laboratory test values outside the reference ranges were flagged and evaluated for clinical significance.

Conclusions:

Pharmacokinetic Results: Mean±SD pharmacokinetic parameters are listed below in Table 11.

TABLE 11

| | Regimen A^{@} | | | Regimen B^{@} | |
|---|---|---|---|---|---|
| | Study Day 1 | Study Day 12 | Study Day 13 | Study Day 1 | Study Day 12 and 13 |
| N | 19 | 19 | 19 | 19 | 19 |
| Cₘₐₓ (ng/mL) | 0.100 ± 0.033 | 0.112 ± 0.031 | 0.105 ± 0.035 | 0.200 ± 0.062 | 0.217 ± 0.077 |
| Cₘₐₓ/Dose | 0.025 ± 0.008 | 0.028 ± 0.008 | 0.026 ± 0.009 | 0.025 ± 0.008 | 0.027 ± 0.010 |
| Cₘᵢₙ (ng/mL)^{&} | ND | 0.005 ± 0.010 | 0.001 ± 0.005 | ND | 0.000 ± 0.000 |
| Tₘₐₓ (h) | 4.8 ± 2.8 | 4.4 ± 2.6 | 4.5 ± 1.6 | 4.4 ± 1.4 | 4.3 ± 2.6 |
| AUC₀₋ₜ | 0.701 ± 0.339 | ND | ND | 2.047 ± 0.881 | ND |
| AUC_{0-∞} | 1.107 ± 0.342^{Φ} | ND | ND | 2.551 ± 1.079 | ND |
| AUC_{0-τ}* | ND | 1.074 ± 0.359 | 0.904 ± 0.196^{#} | ND | 2.257 ± 0.566 |
| AUC/Dose^{†} | 0.277 ± 0.086 | 0.268 ± 0.090 | 0.226 ± 0.049^{%} | 0.319 ± 0.135 | 0.282 ± 0.071 |
| t_{1/2} (h)^{$} | 6.2 ± 2.1^{Φ} | 6.8 ± 3.1^{ε} | 5.8 ± 2.5^{ε} | 7.3 ± 3.2 | 6.8 ± 2.2^{δ} |
| CL/F (L/h)^{&} | 3.95 ± 1.19^{Φ} | 4.33 ± 2.08 | 4.65 ± 1.16 | 3.65 ± 1.61 | 3.79 ± 1.07 |
| Vd_{β}/F (L)^{&} | 38.9 ± 18.7^{Φ} | 44.9 ± 23.0^{ε} | 43.7 ± 17.9^{ε} | 41.0 ± 12.7 | 42.1 ± 28.7^{δ} |
| AI | ND | 1.759 ± 1.829 | 1.483 ± 1.442 | ND | 1.000 ± 0.312 |
| DFL | ND | 2.584 ± 0.889^{‡} | 2.761 ± 0.761^{‡} | ND | 4.637 ± 1.397 |

AUC units: (ng · h/mL),
Cₘₐₓ units: (ng/mL).
^{Φ}N = 14, δ: N = 18,
^{ε}N = 17; ND: Not Determined.
*AUC₀₋₂₄ in Regimen A and AUC₀₋₄₈ in Regimen B after multiple doses.
^{#}Statistically significantly different (P < 0.05) from Regimen A Study Day 1, AUC_{0-inf.}
^{$}Harmonic mean ± pseudo-standard deviation; evaluations of t_{1/2} were based on statistical tests on β.
^{&}Parameter was not tested statistically.
^{†}Regimen A and B Study Day 1: AUC_{0-inf}/Dose; Regimen A Study Day 12 and 13: AUC₀₋₂₄/Dose; Regimen B Study Day 12: AUC₀₋₄₈/Dose.
^{@}Regimen A: 4 μg QD and Regimen B: 8 μg TIW.
^{%}Statistically significantly different (P < 0.05) from Regimen B, Study Day 12 and 13 dose-normalized AUC_{0-48.}
^{‡}: Statistically significantly different from Regimen B, Study Day 12 and 13 DFL.

Safety Results: Overall, 60% (12/20) of subjects reported at least one treatment-emergent adverse event during the entire study. Eight out of 19 (42.1%) in Regimen A and eight out of 19 (42.1%) in Regimen B reported at least one treatment-emergent adverse event during the entire study. Treatment-emergent adverse events reported by two or more subjects in either regimen were eructation, nausea and rhinitis (Regimen A, 10.5% [2/19] subjects each) and asthenia and pharyngitis (Regimen B, 10.5% [2/19] subjects and 15.8% [3/19] subjects, respectively). All remaining adverse events were reported by 5.3% of subjects for each regimen (one subject).

Of the 12 subjects who reported treatment-emergent adverse events across either regimen, 10 reported adverse events which were judged by the investigator to be possibly related to study drug and 1 subject each reported adverse events that were judged as probably not or not related to study drug.

One subject was discontinued from the study due to an adverse event (leukocytosis) that was judged as probably not related to study drug. The severity of all adverse events reported during the study was rated as mild. No severe adverse events were reported during the study. The majority of adverse events resolved without medical intervention. One possibly related event and one probably not related event (a sore throat reported by two subjects) required treatment with warm saline gargles. One probably not related event (rash) and two not related events (athlete's foot and impacted cerumen) required treatment with medication.

Conclusions:

As a result of this study, the following can be concluded: (1) The pharmacokinetics of paricalcitol were dose proportional following single dose and at steady state over the studied dose range; (2) The pharmacokinetics of paricalcitol did not change over time; (3) The steady state for paricalcitol was reached by Study Day 7, and quite possibly much earlier for the studied regimens considering the short half-life of paricalcitol; (4) No unexpected paricalcitol accumulation following multiple-dose administration was observed; (5) Paricalcitol administered orally either as 4 μg QD or 8 μg TIW resulted in similar steady-state exposure; (6) The AUC over the 0 to 48 hr interval following the 8 μg dose on Study Day 12 for TIW regimen was similar to the combined AUC over the two 0 to 24 hour intervals following the 4 μg doses on Study Day 12 and Study Day 13 for QD regimen; (7) The accumulation index (AI) was similar between the QD and TIW regimens; and (8) The DFL for the QD regimen was smaller than that for TIW regimen. Both Regimen A and Regimen B were generally well tolerated by the subjects. No clinically significant physical examination results, vital signs, or ECGs were observed during the course of the study and no clinically significant differences were seen between regimens in their adverse event profiles. There was no apparent trend in any of the safety variables associated with any of the paricalcitol dosing regimens studied. There were no apparent differences between the regimens with respect to safety.

### EXAMPLE 6

### Safety and Efficacy of Oral Formulations of Paricalcitol in Subjects with Pre-End Stage Renal Disease

In this example, a study was conducted to determine the safety and efficacy of oral formulations of paricalcitol as compared to placebo in reducing elevated serum parathyroid hormone (PTH) levels in subjects with pre-end stage renal disease (CKD Stages 3 and 4).

Methodology: This was a Phase 3, prospective, randomized, placebo-controlled, double-blind, 24-week Treatment Phase, multi-center study in CKD (Stages 3 and 4) subjects with elevated PTH levels (≧150 pg/mL). Subjects were randomized in an equal ratio (1:1) to 1 of 2 treatment groups: Paricalcitol Capsule (Group 1) and placebo (Group 2). Potential subjects underwent procedures to determine their baseline intact PTH (iPTH), calcium, and phosphorus levels for eligibility to receive treatment. Subjects who qualified for entry into the Treatment Phase used these results as baseline values against which initial dosing was selected.

The study was performed in 4 parts: a Screening Visit, a Pre-Treatment Phase, a Treatment Phase, and a Follow-Up Phase. At the Screening Visit, subjects reviewed and signed the informed consent form prior to the conduct of any study-specific screening procedures. A spot urine sample was used to calculate calcium/creatinine ratio. A blood sample was drawn for iPTH, blood urea nitrogen (BUN), albumin and serum creatinine levels. Subjects must not have been on active vitamin D therapy for at least 4 weeks and must have had an iPTH value of ε 120 pg/mL to enter the Pre-Treatment Phase. The serum creatinine, BUN, and albumin values were used to calculate the subject's estimated glomerular filtration rate (eGFR) using a formula derived from the “Modification of Diet in Renal Disease” (MDRD) study. Subjects with a calculated eGFR of 15 to 60 mL/min were eligible to undergo Pre-Treatment Phase procedures. The Pre-Treatment Phase was 1 to 4 weeks. During this phase, subjects had 2 scheduled office visits. The office visits could have occurred at any time over a 4-week period but must have been at least 1 day apart. During these visits, subjects were to meet laboratory criteria regarding serum iPTH, calcium, and phosphorus levels. If the subject was unable to meet these criteria, he or she may have been re-screened once after 4 weeks. A 24-hour urine collection for calcium, phosphorus, and creatinine clearance (Ccr) was to be done at either Pre-Treatment Visit 1 or 2. Subjects who satisfied inclusion and exclusion criteria after a minimum of 1 week in the Pre-Treatment Phase were eligible to enter the Treatment Phase. During the Treatment Phase, subjects were to self-administer study drug 3 times weekly, on Monday, Wednesday and Friday, for a total of 24 weeks. The initial dose was 2 or 4 mcg (depending on baseline iPTH levels). Procedures to be performed during the Treatment Phase included vital signs, chemistry and hematology, urinary pyridinoline, urinary deoxypyridinoline, serum bone-specific alkaline phosphatase, serum osteocalcin, urinalysis, spot urine for calcium/creatinine ratio, and recording of adverse events and concurrent medications. Serum iPTH, calcium, phosphorus, and albumin were measured every 2 weeks. Dose adjustments were to be made according to these chemistry results for iPTH, calcium, and phosphorus. Doses may have been increased in 2 mcg increments every 4 weeks. Dose reductions were to occur according to a protocol-specified algorithm. However, dosing could have been adjusted any time if, in the judgment of the Investigator, a risk to subject safety existed.

After Treatment Week 24 (or following premature termination), subjects entered the Follow-Up Phase.

Subjects were to return for study procedures at the Follow-Up Visit 2 to 7 days after their last dose of study drug, and must not have re-started any vitamin D treatment until after the Follow-Up Visit was complete. Throughout the course of the study, safety was evaluated through adverse events, laboratory assessments, and vital signs.

Number of Subjects (Planned and Analyzed):

Planned: 68 subjects (34 per treatment group)

Enrolled: 75 subjects (39 Paricalcitol, 36 Placebo)

| Analyzed: | Paricalcitol | Placebo |
|---|---|---|
| Randomized and Treated | 39 | 36 |
| Evaluated for Primary Efficacy (Intent to Treat) | 36 | 34 |
| Evaluated for Safety and Secondary Efficacy | 39 | 36 |
| (All Treated) | | |

Diagnosis and Main Criteria for Inclusion:

Male or female subjects ≧18 years of age who had been in the care of a physician ≧2 months for CKD prior to entry into the study and had not been on active vitamin D therapy for at least 4 weeks prior to the Screening Visit were eligible. Prior to entry into the Pre-Treatment Phase, subjects had to have iPTH ≧120 pg/mL and an eGFR of 15 to 60 mL/min (and not expected to begin dialysis for at least 6 months). Prior to treatment, subjects had to have an average of 2 consecutive iPTH values of ≧150 pg/mL, taken at least 1 day apart (all values must have been ≧120 pg/mL), 2 consecutive serum calcium levels of ≧8.0 to ≦10.0 mg/dL, and 2 consecutive serum phosphorus levels of ≦5.2 mg/dL. Female subjects of childbearing potential had to have a negative pregnancy test prior to treatment, had to use a protocol specified birth control method throughout the study, and could not be nursing. Subjects who had been taking a phosphate binder were to have been on a stable regimen at least 4 weeks prior to the Screening Visit. Subjects were excluded for the following reasons:
history of an allergic reaction or significant sensitivity to drugs similar to the study drug.
acute renal failure within 12 weeks of the study.
chronic gastrointestinal disease, which, in the Investigator's opinion, may have caused significant gastrointestinal malabsorption.
a spot urine result demonstrating a urine calcium-to-urine creatinine ratio of >0.2 or history of renal stones.
use of aluminum-containing phosphate binders within the last 12 weeks prior to screening or required such medication >3 weeks during the course of the study.
current malignancy or clinically significant liver disease.
an active granulomatous disease (e.g., tuberculosis, sarcoidosis).
history of drug or alcohol abuse within 6 months prior to the Screening Visit.
evidence of poor compliance with diet or medication that, in the Investigator's opinion, may have interfered with adherence to the protocol.
receipt of any investigational drug or participation in any device trial within 30 days prior to study drug administration.
use of maintenance calcitonin, bisphosphonates, or drugs that may have affected calcium or bone metabolism, other than females on stable estrogen and/or progestin therapy.
use of glucocorticoids for a period of >14 days within the last 6 months.
considered by the Investigator to be an unsuitable candidate to receive study drug or to put at risk by study procedures for any reason.
known to be HIV positive.
   Dose/Strength/Concentration and Mode of Administration:

Test product: Paricalcitol 2 mcg soft elastic capsules

Dose: The initial dose was 2 or 4 mcg (depending on baseline iPTH levels [≦500 pg/mL=2 mcg, >500 pg/mL=4 mcg])

Mode of administration: oral

Duration of Treatment: 24 weeks

Reference Therapy, Dose and Mode of Administration:

Placebo, identical in appearance to Paricalcitol capsules.

Mode of administration: oral

Criteria for Evaluation:

Efficacy: The primary efficacy endpoint was the achievement of 2 consecutive >30% decreases from baseline iPTH levels.

The secondary efficacy analyses include change and percent change from baseline analyses in iPTH and change from baseline analyses in biochemical bone activity markers.

Safety: Safety was assessed through an evaluation of clinically meaningful hypercalcemia (2 consecutive calcium results >10.5 mg/dL). Additionally, safety was assessed by the incidence of adverse events, the change from baseline in chemistry, hematology and urinalysis laboratory variables, the change from baseline in subject vital signs, and progressive changes in renal function observed via changes in eGFR.

Statistical Methods: All statistical hypothesis tests performed were two-tailed and p-values ≦0.05 were considered statistically significant.

Efficacy:

The Intent-To-Treat population (Full Analysis Set) was defined as all randomized subjects with a baseline iPTH and at least 2 on-treatment iPTH measurements. This population was used in the primary efficacy analysis.

The primary efficacy analysis was a comparison between the paricalcitol and placebo treatment groups of the proportion of subjects achieving 2 consecutive decreases from baseline in iPTH of at least 30%. This comparison was performed using a Fisher's exact test. All randomized subjects who received at least 1 dose of study drug were used in secondary efficacy analyses. Secondary efficacy analyses were performed comparing changes/percent change from baseline between the paricalcitol and placebo treatment groups using a one-way analysis of variance (ANOVA) with treatment group as the factor for the following variables: iPTH and biochemical bone activity markers.

Safety:

All randomized subjects who received at least 1 dose of study drug were used in safety analyses. The primary safety analysis was a comparison between the paricalcitol and placebo treatment groups of the proportion of subjects achieving clinically meaningful hypercalcemia (2 consecutive calcium measurements >10.5 mg/dL). This comparison was performed using a Fisher's exact test. Secondary safety analyses were performed comparing changes/percent changes from baseline between the paricalcitol and placebo treatment groups using a one-way ANOVA with treatment group as the factor for the following variables: hematology, complete chemistry, and urinalysis variables; 24-hour urine collections, eGFR, urinary calcium/creatinine ratio, cardiovascular markers, and vital signs. Secondary safety analyses also consisted of an analysis of “treatment-emergent” adverse events (i.e., adverse events with an onset date on or after the date the first dose of study drug was taken). Adverse events were summarized by body system and COSTART term according to the COSTART V adverse event-coding dictionary. Comparisons of the percentage of subjects experiencing an adverse event between the paricalcitol and placebo treatment groups were performed using a Fisher's exact test.

Conclusions:

Efficacy Results:

A statistically significantly (p<0.001) greater proportion of subjects treated with paricalcitol (initially dosed according to baseline iPTH values) had 2 consecutive >30% decreases from baseline in iPTH compared with subjects who received placebo (33/36, 92% versus 4/34, 12%). Additionally, in an exploratory analysis to evaluate the robustness of the primary efficacy analysis, a statistically significantly (p<0.001) greater proportion of paricalcitol subjects had 4 consecutive ≧30% decreases from baseline in iPTH compared with placebo subjects (26/36, 72% versus 0/34, 0%). There was a statistically significant difference between the paricalcitol and placebo treatment groups in mean change from baseline to Final Visit in iPTH using ANOVA with treatment as the factor. paricalcitol capsule-treated subjects had a mean decrease (−58.1 pg/mL, representing a 19.2% decrease) in iPTH at the Final Visit compared with a mean increase (50.4 pg/mL, representing a 16.9% increase) among placebo-treated subjects. Similarly, paricalcitol-treated subjects had a statistically significant mean decrease (−95.7 pg/mL, representing a 33.0% decrease) in iPTH at the Last On-Treatment Visit compared with a mean increase (32.5 pg/mL, representing a 11.2% increase) among placebo-treated subjects. The larger mean decrease and mean percent decrease using the Last On-Treatment Visit may be more representative of a treatment effect. Statistically significant differences were observed between the paricalcitol and placebo treatment groups at all scheduled visits of the Treatment Phase for both change and percent change from baseline in iPTH.

In paricalcitol-treated subjects, decreases in iPTH were observed as early as Week 3 (the first time iPTH was measured after the first dose) and continued throughout the Treatment Phase. A 30% mean reduction in iPTH occurred by Week 9 and the maximum decrease (−46.0%) from baseline in iPTH was observed at Week 19. Using a one-way ANOVA, statistically significant differences were observed between the paricalcitol and placebo treatment groups in mean changes from baseline to Final Visit for all of the biochemical bone activity marker variables. Paricalcitrol-treated subjects had mean decreases in urinary deoxypyridinoline, urinary pyridinoline, serum osteocalcin, and serum bone-specific alkaline phosphatase while placebo subjects experienced mean increases in urinary deoxypyridinoline, urinary pyridinoline, and serum osteocalcin and a small mean decrease in serum bone-specific alkaline phosphatase. The one-way ANOVA for urinary pyridinoline yielded a statistically significant difference in change from baseline between the paricalcitol and placebo treatment groups (p=0.043) while the Wilcoxon rank-sum test yielded a non-significant difference for changes from baseline between the 2 treatment groups (p=0.138). The results of the Wilcoxon rank-sum tests for the other bone activity markers were consistent with the results using the one-way ANOVA. The favorable result observed in the paricalcitol group suggests correction of high-turnover bone disease associated with 2° HPT.

Safety Results:

No statistically significant differences were observed between the treatment groups for the overall incidence of adverse events or for the incidence of any specific adverse event. Treatment-emergent adverse events were experienced by 79% of paricalcitol subjects and 64% of placebo subjects. The majority of the adverse events reported in either treatment group were mild or moderate in severity (88% paricalcitol and 88% placebo) and considered by the Investigator not related to study drug administration (85% paricalcitol and 91% placebo). The most commonly reported adverse events in the paricalcitol group were pain, pharyngitis, viral infection (10% each), constipation, depression, headache, hypertension, infection, rhinitis, and vertigo (8% each). The most commonly reported adverse events in the placebo group were uremia (11%), pharyngitis, accidental injury, CHF, and peripheral edema (8% each). One (1) paricalcitol subject died due to cardiopulmonary arrest that was considered not related to study drug. Overall, 18 subjects (9 paricalcitol and 9 placebo) reported serious adverse events, including the 1 death, during the Treatment and Follow-Up Phases of the study. Only 1 serious adverse event (elevated liver enzymes reported for 1 paricalcitol subject) was considered by the Investigator to have a causal relationship to study drug (possibly related). Three (3) subjects (1 paricalcitol and 2 placebo) were listed as having terminated prematurely from the study due to adverse events. The only event leading to premature termination considered by the Investigator to have a causal relationship to study drug was elevated liver enzymes, which was reported for the paricalcitol subject. This event also was reported as a serious adverse event. No statistically significant differences were observed between the treatment groups in the proportion of subjects who developed clinically meaningful hypercalcemia, defined as at least 2 consecutive calcium values >10.5 mg/dL (1/38 paricalcitol subjects, 0/35 placebo subjects). No statistically significant differences were observed between the treatment groups in mean change from baseline to Final Visit or Last On-Treatment Visit in calcium, Ca×P, and phosphorus. Both treatment groups experienced small mean increases from baseline in calcium, Ca×P, and phosphorus. Except for Weeks 11, 19, and 21, an effect of paricalcitol on calcium was not detected, since no statistically significant difference between the paricalcitol and placebo groups was observed at any other scheduled visit of the Treatment Phase. No statistically significant differences were observed between the treatment groups for mean changes from baseline to any of the scheduled visits of the Treatment Phase for phosphorus or Ca×P. Statistically significant differences were observed between the treatment groups in nonfasting triglycerides and alkaline phosphatase. A mean increase from baseline in nonfasting triglycerides was observed in the paricalcitol treatment group, with a mean decrease from baseline in the placebo group; however, the difference between the treatment groups in mean change from baseline in cholesterol was not statistically significant. Given the nonfasting state during which laboratory values were measured, the difference between the treatment groups in triglycerides was not considered clinically meaningful. A statistically significant mean decrease from baseline in alkaline phosphatase was observed in the paricalcitol treatment group compared with a mean increase from baseline in the placebo group. A decrease in alkaline phosphatase parallels the decrease in bone-specific alkaline phosphatase supporting improvement in the bone abnormalities associated with 2° HPT. No statistically significant differences were observed between the treatment groups in mean change and mean percent change from baseline to Final Visit in eGFR and creatinine for all subjects who completed 24 weeks of treatment. Additionally, no statistically significant difference was observed between the treatment groups in mean change from baseline to Final Visit in 24-hour urine collection variables (calcium, phosphorus, Ccr) or urinary calcium/creatinine ratio. Therefore, a paricalcitol treatment effect was not detected for urinary calcium and phosphorus excretion as well as kidney function parameters (eGFR, Ccr, and serum creatinine). Evaluations of other laboratory analyses, vital signs and physical examinations revealed no clinically meaningful changes as a result of paricalcitol treatment.

Conclusions:

Paricalcitol capsule is safe and well tolerated for the treatment and prevention of 2° HPT in CKD (Stages 3 and 4) subjects. Paricalcitol capsule is effective for the treatment and prevention of 2° HPT in CKD (Stages 3 and 4) subjects. When paricalcitol capsule was initially dosed according to severity of the 2° HPT, a statistically significantly (p<0.001) greater proportion of subjects had 2 consecutive >30% decreases from baseline in iPTH compared with subjects who received placebo (33/36, 92% versus 4/34, 12%). Statistically significant differences were observed between the paricalcitol and placebo treatment groups at all scheduled visits of the Treatment Phase for both change and percent change from baseline in iPTH. In paricalcitol-treated subjects, decreases in iPTH were observed as early as Week 3 (the first time iPTH was measured after the first dose). Clinically meaningful suppression of iPTH (a 30% decrease from baseline in iPTH) was achieved within 9 weeks of treatment and was observed throughout the Treatment Phase. Serum alkaline phosphatase and biochemical bone markers, which are used commonly to monitor bone remodeling activity in patients with metabolic bone disease, were reduced significantly in paricalcitol capsule treated subjects compared to placebo-treated subjects. The favorable result observed in the paricalcitol group suggests correction of high-turnover bone disease associated with 2° HPT. No statistically significant differences were observed between the treatment groups in the proportion of subjects who developed clinically meaningful hypercalcemia (2 consecutive calcium values >10.5 mg/dL), or in mean changes from baseline to any of the scheduled visits of the Treatment Phase or to the Final Visit for phosphorus or Ca×P. No statistically significant differences were observed between the treatment groups in mean change and percent changes from baseline to Final Visit in eGFR or creatinine. Additionally, no statistically significant differences were observed between the treatment groups in mean change from baseline to Final Visit in 24-hour urine collection variables (calcium, phosphorus, Ccr) or urinary calcium/creatinine ratio. Therefore, a paricalcitol treatment effect was not detected for urinary calcium and phosphorus excretion as well as kidney function parameters (eGFR, Ccr, and serum creatinine).

### EXAMPLE 7

### Additional Studies of the Safety and Efficacy of Oral Formulations of Paricalcitol in Subjects with CKD (Stages 3 and 4)

In this example, a further study was conducted to determine the safety and efficacy of oral formulations of paricalcitol as compared to placebo in reducing elevated serum parathyroid hormone (PTH) levels in subjects with CKD (stages 3 and 4).

Methodology: This was a Phase 3, prospective, randomized, placebo-controlled, double-blind, 24-week Treatment Phase, multi-center study in CKD (Stages 3 and 4) subjects with elevated PTH levels (≧150 pg/mL). Subjects were randomized in an equal ratio (1:1) to 1 of 2 treatment groups: paricalcitol capsule (Group 1) and placebo (Group 2). Potential subjects underwent procedures to determine their baseline intact PTH (iPTH), calcium, and phosphorus levels for eligibility to receive treatment. Subjects who qualified for entry into the Treatment Phase used these results as baseline values against which initial dosing was selected.

The study was performed in 4 parts: a Screening Visit, a Pre-Treatment Phase, a Treatment Phase, and a Follow-Up Phase. At the Screening Visit, subjects reviewed and signed the informed consent form prior to the conduct of any study-specific screening procedures. A spot urine sample was used to calculate calcium/creatinine ratio. A blood sample was drawn for iPTH, blood urea nitrogen (BUN), albumin and serum creatinine levels. Subjects must not have been on active vitamin D therapy for at least 4 weeks and must have had an iPTH value of ≧120 pg/mL to enter the Pre-Treatment Phase. The serum creatinine, BUN, and albumin values were used to calculate the subject's estimated glomerular filtration rate (eGFR) using a formula derived from the “Modification of Diet in Renal Disease” (MDRD) study. Subjects with a calculated eGFR of 15 to 60 mL/min were eligible to undergo Pre-Treatment Phase procedures.

The Pre-Treatment Phase was 1 to 4 weeks. During this phase, subjects had 2 scheduled office visits. The office visits could have occurred at any time over a 4-week period but must have been at least 1 day apart. During these visits, subjects were to meet laboratory criteria regarding serum iPTH, calcium, and phosphorus levels. If the subject was unable to meet these criteria, he or she may have been re-screened once after 4 weeks. A 24-hour urine collection for calcium, phosphorus, and creatinine clearance (Ccr) was to be done at either Pre-Treatment Visit 1 or 2. Subjects who satisfied inclusion and exclusion criteria after a minimum of 1 week in the Pre-Treatment Phase were eligible to enter the Treatment Phase.

During the Treatment Phase, subjects were to self-administer study drug 3 times weekly, on Monday, Wednesday and Friday, for a total of 24 weeks. The initial dose was 2 or 4 mcg (depending on baseline iPTH levels). Procedures to be performed during the Treatment Phase included vital signs, chemistry and hematology, urinary pyridinoline, urinary deoxypyridinoline, serum bone-specific alkaline phosphatase, serum osteocalcin, urinalysis, spot urine for calcium/creatinine ratio, and recording of adverse events and concurrent medications. Serum iPTH, calcium, phosphorus, and albumin were measured every 2 weeks. Dose adjustments were to be made according to these chemistry results for iPTH, calcium, and phosphorus. Doses may have been increased in 2 mcg increments every 4 weeks.

Dose reductions were to occur according to a protocol-specified algorithm. However, dosing could have been adjusted any time if, in the judgment of the Investigator, a risk to subject safety existed.

After Treatment Week 24 (or following premature termination), subjects entered the Follow-Up Phase.

Subjects were to return for study procedures at the Follow-Up Visit 2 to 7 days after their last dose of study drug, and must not have re-started any vitamin D treatment until after the Follow-Up Visit was complete.

Throughout the course of the study, safety was evaluated through adverse events, laboratory assessments, and vital signs.

Number of Subjects (Planned and Analyzed):

Planned: 68 subjects (34 per treatment group)

Enrolled: 70 subjects (33 Oral paricalcitol, 37 Placebo)

| Analyzed: | Paricalcitol | Placebo |
|---|---|---|
| Randomized and Treated | 33 | 37 |
| Evaluated for Primary | 32 | 36 |
| Efficacy (Intent-to-Treat) | | |
| Evaluated for Safety and | 33 | 37 |
| Secondary Efficacy (All Treated) | | |

Diagnosis and Main Criteria for Inclusion:

Male or female subjects ≧18 years of age who had been in the care of a physician ≧2 months for CKD prior to entry into the study and had not been on active vitamin D therapy for at least 4 weeks prior to the Screening Visit were eligible. Prior to entry into the Pre-Treatment Phase, subjects had to have iPTH ≧120 pg/mL and an eGFR of 15 to 60 mL/min (and not expected to begin dialysis for at least 6 months).

Prior to treatment, subjects had to have an average of 2 consecutive iPTH values of ≧150 pg/mL, taken at least 1 day apart (all values must have been ≧120 pg/mL), 2 consecutive serum calcium levels of ≧8.0 to ≦10.0 mg/dL, and 2 consecutive serum phosphorus levels of ≦5.2 mg/dL. Female subjects of childbearing potential had to have a negative pregnancy test prior to treatment, had to use a protocol specified birth control method throughout the study, and could not be nursing. Subjects who had been taking a phosphate binder were to have been on a stable regimen at least 4 weeks prior to the Screening Visit.

Subjects were excluded for the following reasons:
history of an allergic reaction or significant sensitivity to drugs similar to the study drug.
acute renal failure within 12 weeks of the study.
chronic gastrointestinal disease, which, in the Investigator's opinion, may have caused significant gastrointestinal malabsorption.
a spot urine result demonstrating a urine calcium-to-urine creatinine ratio of >0.2 or history of renal stones.
use of aluminum-containing phosphate binders within the last 12 weeks prior to screening or required such medication >3 weeks during the course of the study.
current malignancy or clinically significant liver disease.
an active granulomatous disease (e.g., tuberculosis, sarcoidosis).
history of drug or alcohol abuse within 6 months prior to the Screening Visit.
evidence of poor compliance with diet or medication that, in the Investigator's opinion, may have interfered with adherence to the protocol.
receipt of any investigational drug or participation in any device trial within 30 days prior to study drug administration.
use of maintenance calcitonin, bisphosphonates, or drugs that may have affected calcium or bone metabolism, other than females on stable estrogen and/or progestin therapy.
use of glucocorticoids for a period of >14 days within the last 6 months.
considered by the Investigator to be an unsuitable candidate to receive study drug or to put at risk by study procedures for any reason.
known to be HIV positive.
   Test Product, Dose/Strength/Concentration and Mode of Administration:

Test product: Paricalcitol 2 mcg soft elastic capsules

Dose: The initial dose was 2 or 4 mcg (depending on baseline iPTH levels [≦500 pg/mL=2 mcg, >500 pg/mL=4 mcg])

Mode of administration: oral

Duration of Treatment: 24 weeks

Reference Therapy, Dose and Mode of Administration:

Placebo, identical in appearance to paricalcitol capsules.

Mode of administration: oral

Criteria for Evaluation:

Efficacy: The primary efficacy endpoint was the achievement of 2 consecutive ≧30% decreases from baseline iPTH levels. The secondary efficacy analyses include change and percent change from baseline analyses in iPTH and change from baseline analyses in biochemical bone markers.

Safety: Safety was assessed through an evaluation of clinically meaningful hypercalcemia (2 consecutive calcium results >10.5 mg/dL). Additionally, safety was assessed by the incidence of adverse events, the change from baseline in chemistry, hematology and urinalysis laboratory variables, the change from baseline in subject vital signs, and progressive changes in renal function observed via changes in eGFR.

Statistical Methods: All statistical hypothesis tests performed were two-tailed and p-values ≦0.05 were considered statistically significant.

Efficacy:

The Intent-To-Treat population (Full Analysis Set) was defined as all randomized subjects with a baseline iPTH and at least 2 on-treatment iPTH measurements. This population was used in the primary efficacy analysis.

The primary efficacy analysis was a comparison between the paricalcitol and placebo treatment groups of the proportion of subjects achieving 2 consecutive decreases from baseline in iPTH of at least 30%.

This comparison was performed using a Fisher's exact test.

All randomized subjects who received at least 1 dose of study drug were used in secondary efficacy analyses.

Secondary efficacy analyses were performed comparing changes/percent change from baseline between the paricalcitol and placebo treatment groups using a one-way ANOVA with treatment group as the factor for the following variables: iPTH and biochemical bone markers.

Safety:

All randomized subjects who received at least 1 dose of study drug were used in safety analyses. The primary safety analysis was a comparison between the paricalcitol and placebo treatment groups of the proportion of subjects achieving clinically meaningful hypercalcemia (2 consecutive calcium measurements >10.5 mg/dL). This comparison was performed using a Fisher's exact test. Secondary safety analyses were performed comparing changes/percent changes from baseline between the paricalcitol and placebo treatment groups using a one-way ANOVA with treatment group as the factor for the following variables: hematology, complete chemistry, and urinalysis variables; 24-hour urine collections, eGFR, urinary calcium/creatinine ratio, cardiovascular markers, and vital signs. Secondary safety analyses also consisted of an analysis of “treatment-emergent” adverse events (i.e., adverse events with an onset date on or after the date the first dose of study drug was taken). Adverse events were summarized by body system and COSTART term according to the COSTART V adverse event-coding dictionary. Comparisons of the percentage of subjects experiencing an adverse event between the Oral paricalcitol and placebo treatment groups were performed using a Fisher's exact test.

Conclusions:

Efficacy Results:

A statistically significantly (p<0.001) greater proportion of subjects treated with oral paricalcitol (initially dosed according to baseline iPTH values) had 2 consecutive ≧30% decreases from baseline in iPTH compared with subjects who received placebo (29/32, 91% versus 6/36, 17%). Additionally, in an exploratory analysis to evaluate the robustness of the primary efficacy analysis, a statistically significantly (p<0.001) greater proportion of paricalcitol subjects had 4 consecutive ≧30% decreases from baseline in iPTH compared with placebo subjects (26/32, 81% versus 0/36, 0%).

There was a statistically significant difference between the oral paricalcitol and placebo treatment groups in mean change from baseline to Final Visit in iPTH using ANOVA with treatment as the factor. Paricalcitol-treated subjects had a mean decrease (−80.7 pg/mL, representing a 30.3% decrease) in iPTH at the Final Visit compared with a mean increase (12.2 pg/mL, representing a 9.4% increase) among placebo-treated subjects. Similarly, oral paricalcitol-treated subjects had a statistically significant mean decrease (−83.1 pg/mL, representing a 33.4% decrease) in iPTH at the Last On-Treatment Visit compared with a mean increase (10.1 pg/mL, representing a 2.9% increase) among placebo-treated subjects. Statistically significant differences were observed between the oral paricalcitol and placebo treatment groups at all scheduled visits of the Treatment Phase for both change and percent change from baseline in iPTH. In oral paricalcitol-treated subjects, decreases in iPTH were observed as early as Week 3 (the first time iPTH was measured after the first dose) and continued throughout the Treatment Phase. A 30% mean reduction in iPTH occurred by Week 9 and the maximum decrease (−48.2%) from baseline in iPTH was observed at Week 17. Statistically significant differences were observed between the oral paricalcitol and placebo treatment groups in mean changes from baseline to Final Visit for the serum biochemical bone activity markers of serum osteocalcin and serum bone-specific alkaline phosphatase. Oral paricalcitol-treated subjects had mean decreases in serum osteocalcin and serum bone-specific alkaline phosphatase while placebo subjects experienced a mean increase in serum osteocalcin and a small mean decrease in serum bone-specific alkaline phosphatase. Serum bone-specific alkaline phosphorus and osteocalcin are currently considered more sensitive and specific bone markers to evaluate the degree of bone remodeling in the setting of CKD than urine bone markers. The favorable result observed in the oral paricalcitol group suggests correction of high-turnover bone disease associated with 2° HPT.

Safety Results:

No statistically significant differences were observed between the treatment groups for the overall incidence of adverse events or for the incidence of any specific adverse event. Treatment-emergent adverse events were experienced by 76% of paricalcitol subjects and 78% of placebo subjects. The majority of the adverse events reported in both treatment groups were mild or moderate in severity (96% oral paricalcitol and 97% placebo) and considered by the Investigator to be not related to study drug administration (81% oral paricalcitol and 80% placebo). The most commonly reported adverse events in the oral paricalcitol group were hypotension, uremia, dizziness (12% each), diarrhea and edema (9% each). The most commonly reported adverse events in the placebo group were pharyngitis and gout (11%) oral paricalcitol capsule. One (1) placebo subject died due to a cardiac arrest that was considered not related to study drug. Overall, 8 subjects (6 oral paricalcitol and 2 placebo) reported serious adverse events, including the 1 death, during the Treatment and Follow-Up Phases of the study. None of the serious adverse events was considered by the Investigator to have a causal relationship to study drug. Two (2) subjects (1 paricalcitol and 1 placebo) were listed as having terminated prematurely from the study due to adverse events. None of the events leading to termination was considered by the Investigator to have a causal relationship to study drug. No statistically significant differences were observed between the treatment groups in the proportion of subjects who developed clinically meaningful hypercalcemia, defined as at least 2 consecutive calcium values >10.5 mg/dL (1/33 oral paricalcitol subjects, 0/36 placebo subjects). No statistically significant differences were observed between the treatment groups in mean change from baseline to Final Visit in calcium, phosphorus, and Ca×P. Both treatment groups experienced small mean increases from baseline in calcium, phosphorus, and Ca×P. For serum calcium, mean change from baseline to Last On-Treatment Visit was statistically significantly different between the treatment groups; a small mean increase in calcium was observed in the paricalcitol group (0.19 mg/dL) and a small mean decrease was observed in the placebo group (−0.14 mg/dL), suggesting a minimal effect of treatment on serum calcium. The mean decrease in calcium observed in the placebo group is consistent with the pathogenesis of 2° HPT and reflects the disease state in this subject population. Although statistically significant, the minimal difference between the paricalcitol and placebo group in calcium is not considered clinically significant. Mean changes from baseline to Last On-Treatment Visit in phosphorus or Ca×P were not statistically significantly different between the 2 treatment groups. Statistically significant differences between the treatment groups were observed at every scheduled visit of the Treatment Phase for mean change from baseline in calcium values, with small mean increases in calcium values observed in the paricalcitol group and small mean decreases in calcium values observed in the placebo group. Mean calcium values ranged from 9.36 to 9.62 mg/dL in the paricalcitol group and 9.32 to 9.49 mg/dL in the placebo group. No statistically significant differences were observed between the treatment groups for mean changes from baseline to any of the scheduled visits of the Treatment Phase for phosphorus or Ca×P.

A statistically significant difference was observed between the treatment groups in mean change from baseline to Final Visit in alkaline phosphatase. A mean decrease from baseline in alkaline phosphatase was observed in the oral paricalcitol treatment group while a mean increase from baseline in this variable was observed in the placebo group. A decrease in alkaline phosphatase parallels the decrease in bone-specific alkaline phosphatase supporting improvement in bone abnormalities associated with 2° HPT. No statistically significant difference was observed between the treatment groups in mean change from baseline to Final Visit in 24-hour urine collection variables (calcium, phosphorus, Ccr) or urinary calcium/creatinine ratio. Therefore, an oral paricalcitol treatment effect was not detected on urinary calcium and phosphorus excretion. The natural course of kidney disease is characterized by the progressive loss of renal function over time. In the ANOVA of eGFR for subjects who completed 24 weeks of treatment, no statistically significant difference was observed for mean change from baseline to Final Visit; however, a statistically significant difference between the treatment groups was observed for mean percent change from baseline (p=0.047). The oral paricalcitol treatment group had larger mean percent decreases in eGFR (−16.61%) compared with the percent decreases observed in the placebo group (−4.64%). Results were similar for the ANOVA and ANCOVA for mean change from baseline to Final Visit in eGFR. After adjusting for baseline differences, no statistically significant difference was observed between the treatment groups for mean percent change in eGFR from baseline to Final Visit (p=0.057). In the ANOVA and ANCOVA of creatinine for subjects who completed 24 weeks of treatment, no statistically significant differences were observed between the treatment groups for mean change or mean percent change from baseline to Final Visit. The Wilcoxon rank-sum test indicated a statistically significant difference between the treatment groups for mean change and mean percent change from baseline to Final Visit in creatinine.

More paricalcitol-treated subjects were taking high ceiling diuretics and ACE inhibitors and/or angiotensin II receptor blockers at baseline compared to placebo-treated subjects. Hypovolemia due to diuretic use may potentiate hypotensive effect and/or renal effect in subjects receiving ACE inhibitors and/or angiotensin II receptor blockers. Four (4) oral paricalcitol-treated subjects and 1 placebo-treated subject experienced an adverse event of hypotension during the study. Evaluations of other laboratory analyses, vital signs and physical examinations revealed no clinically meaningful changes as a result of oral paricalcitol treatment.

Conclusions:

Paricalcitol capsule is safe and well tolerated for the treatment and prevention of 2° HPT in CKD (Stages 3 and 4) subjects.

Paricalcitol capsule is effective for the treatment and prevention of 2° HPT in CKD (Stages 3 and 4) subjects.

When the paricalcitol capsule was initially dosed according to severity of the 2° HPT, a statistically significantly (p<0.001) greater proportion of subjects had 2 consecutive ≧30% decreases from baseline in iPTH compared with subjects who received placebo (29/32, 91% versus 6/36, 17%). Statistically significant differences were observed between the paricalcitol and placebo treatment groups at all scheduled visits of the Treatment Phase for both change and percent change from baseline in iPTH. In the oral paricalcitol-treated subjects, decreases in iPTH were observed as early as Week 3 (the first time iPTH was measured after the first dose). Clinically meaningful suppression of iPTH (a 30% decrease from baseline in iPTH) was achieved within 9 weeks of treatment and was also observed from Week 13 throughout the remainder of the Treatment Phase. Serum alkaline phosphatase and biochemical bone activity markers (serum osteocalcin, and serum bone-specific alkaline phosphatase), which are used commonly to monitor bone remodeling activity in patients with metabolic bone disease, were reduced significantly in oral paricalcitol-treated subjects compared to placebo-treated subjects. The favorable result observed in the oral paricalcitol group suggests correction of high-turnover bone disease associated with 2° HPT.

No statistically significant differences were observed between the treatment groups in the proportion of subjects who developed clinically meaningful hypercalcemia (2 consecutive calcium values >10.5 mg/dL). Mean changes from baseline to any of the scheduled visits of the Treatment Phase, the Final Visit, or the Last On-Treatment Visit for phosphorus or Ca×P were not statistically significant.

No statistically significant differences were observed between the treatment groups in mean change from baseline to Final Visit in 24-hour urine collection variables (calcium, phosphorus, Ccr) or urinary calcium/creatinine ratio. Therefore, an oral paricalcitol treatment effect was not detected on urinary calcium and phosphorus excretion.

### EXAMPLE 8

### Additional Studies of the Safety and Efficacy of Oral Formulations of Paricalcitol in Subjects with CKD (Stages 3 and 4)

In this example, a further study was conducted to determine the safety and efficacy of oral formulations of paricalcitol as compared to placebo in reducing elevated serum parathyroid hormone (PTH) levels in subjects with CKD (stages 3 and 4).

Methodology: This was a Phase 3, prospective, randomized, placebo-controlled, double-blind, 24-week Treatment Phase, multi-center study in CKD (Stages 3 and 4) subjects with elevated PTH levels (≧150 pg/mL). Subjects were randomized in an equal ratio (1:1) to 1 of 2 treatment groups: Paricalcitol capsule (Group 1) and placebo (Group 2). Potential subjects underwent procedures to determine their baseline intact PTH (iPTH), calcium, and phosphorus levels for eligibility to receive treatment. Subjects who qualified for entry into the Treatment Phase used these results as baseline values against which initial dosing was selected.

The study was performed in 4 parts: a Screening Visit, a Pre-Treatment Phase, a Treatment Phase, and a Follow-Up Phase. At the Screening Visit, subjects reviewed and signed the informed consent form prior to the conduct of any study-specific screening procedures. A spot urine sample was used to calculate calcium/creatinine ratio. A blood sample was drawn for iPTH, blood urea nitrogen (BUN), albumin and serum creatinine levels. Subjects must not have been on active vitamin D therapy for at least 4 weeks and must have had an iPTH value of ≧120 pg/mL to enter the Pre-Treatment Phase. The serum creatinine, BUN, and albumin values were used to calculate the subject's estimated glomerular filtration rate (eGFR) using a formula derived from the “Modification of Diet in Renal Disease” (MDRD) study.

Subjects with a calculated eGFR of 15 to 60 mL/min were eligible to undergo Pre-Treatment Phase procedures.

The Pre-Treatment Phase was 1 to 4 weeks. During this phase, subjects had 2 scheduled office visits. The office visits could have occurred at any time over a 4-week period but must have been at least 1 day apart. During these visits, subjects were to meet laboratory criteria regarding serum iPTH, calcium, and phosphorus levels. If the subject was unable to meet these criteria, he or she may have been re-screened once after 4 weeks. A 24-hour urine collection for calcium, phosphorus, and creatinine clearance (Ccr) was to be done at either Pre-Treatment Visit 1 or 2. Subjects who satisfied inclusion and exclusion criteria after a minimum of 1 week in the Pre-Treatment Phase were eligible to enter the Treatment Phase.

During the Treatment Phase, subjects were to self-administer study drug once daily for a total of 24 weeks. The initial dose was 1 or 2 mcg (depending on baseline iPTH levels). Procedures to be performed during the Treatment Phase included vital signs, chemistry and hematology, urinary pyridinoline, urinary deoxypyridinoline, serum bone-specific alkaline phosphatase, serum osteocalcin, urinalysis, spot urine for calcium/creatinine ratio, and recording of adverse events and concurrent medications. Serum iPTH, calcium, phosphorus, and albumin were measured every 2 weeks. Dose adjustments were to be made according to these chemistry results for iPTH, calcium, and phosphorus. Doses may have been increased in 1 mcg increments every 4 weeks. Dose reductions were to occur according to a protocol-specified algorithm. However, dosing could have been adjusted any time if, in the judgment of the Investigator, a risk to subject safety existed.

After Treatment Week 24 (or following premature termination), subjects entered the Follow-Up Phase.

Subjects were to return for study procedures at the Follow-Up Visit 2 to 7 days after their last dose of study drug, and must not have re-started any vitamin D treatment until after the Follow-Up Visit was complete.

Throughout the course of the study, safety was evaluated through adverse events, laboratory assessments, and vital signs.

Number of Subjects (Planned and Analyzed):

Planned: 68 subjects (34 per treatment group)

Enrolled: 75 subjects (35 Paricalcitol, 40 Placebo)

| Analyzed: | Paricalcitol | Placebo |
|---|---|---|
| Randomized and Treated | 35 | 40 |
| Evaluated for Primary | 33 | 38 |
| Efficacy (Intent-to-Treat) | | |
| Evaluated for Safety and | 35 | 40 |
| Secondary Efficacy (All Treated) | | |

Diagnosis and Main Criteria for Inclusion:

Male or female subjects ≧18 years of age who had been in the care of a physician ≧2 months for CKD prior to entry into the study and had not been on active vitamin D therapy for at least 4 weeks prior to the Screening Visit were eligible. Prior to entry into the Pre-Treatment Phase, subjects had to have iPTH≧120 pg/mL and an eGFR of 15 to 60 mL/min (and not expected to begin dialysis for at least 6 months).

Prior to treatment, subjects had to have an average of 2 consecutive iPTH values of ≧150 pg/mL, taken at least 1 day apart (all values must have been ≧120 pg/mL), 2 consecutive serum calcium levels of ≧8.0 to <10.0 mg/dL, and 2 consecutive serum phosphorus levels of ≦5.2 mg/dL. Female subjects of childbearing potential had to have a negative pregnancy test prior to treatment, had to use a protocols specified birth control method throughout the study, and could not be nursing. Subjects who had been taking a phosphate binder were to have been on a stable regimen at least 4 weeks prior to the Screening Visit.

Subjects were excluded for the following reasons:
history of an allergic reaction or significant sensitivity to drugs similar to the study drug.
acute renal failure within 12 weeks of the study.
chronic gastrointestinal disease, which, in the Investigator's opinion, may have caused significant gastrointestinal malabsorption.
a spot urine result demonstrating a urine calcium-to-urine creatinine ratio of >0.2 or history of renal stones.
use of aluminum-containing phosphate binders within the last 12 weeks prior to screening or required such medication >3 weeks during the course of the study.
current malignancy or clinically significant liver disease.
an active granulomatous disease (e.g., tuberculosis, sarcoidosis).
history of drug or alcohol abuse within 6 months prior to the Screening Visit.
evidence of poor compliance with diet or medication that, in the Investigator's opinion, may have interfered with adherence to the protocol.
receipt of any investigational drug or participation in any device trial within 30 days prior to study drug administration.
use of maintenance calcitonin, bisphosphonates, or drugs that may have affected calcium or bone metabolism, other than females on stable estrogen and/or progestin therapy.
use of glucocorticoids for a period of >14 days within the last 6 months.
considered by the Investigator to be an unsuitable candidate to receive study drug or to put at risk by study procedures for any reason.
known to be HIV positive.
   Test Product, Dose/Strength/Concentration and Mode of Administration:

Test product: Paricalcitol 1 mcg soft elastic capsules

Dose: The initial dose was 1 mcg (depending on baseline iPTH levels [≦500 pg/mL=1 mcg, >500 pg/mL=2 mcg])

Mode of administration: oral

Duration of Treatment: 24 weeks

Reference Therapy, Dose and Mode of Administration:

Placebo, identical in appearance to Paricalcitol capsules.

Mode of administration: oral

Criteria for Evaluation:

Efficacy: The primary efficacy endpoint was the achievement of 2 consecutive >30% decreases from baseline iPTH levels.

The secondary efficacy analyses include change and percent change from baseline analyses in iPTH and change from baseline analyses in biochemical bone activity markers.

Safety: Safety was assessed through an evaluation of clinically meaningful hypercalcemia (2 consecutive calcium results >10.5 mg/dL). Additionally, safety was assessed by the incidence of adverse events, the change from baseline in chemistry, hematology and urinalysis laboratory variables, the change from baseline in subject vital signs, and progressive changes in renal function observed via changes in eGFR.

Statistical Methods: All statistical hypothesis tests performed were two-tailed and p-values ≦0.05 were considered statistically significant.

Efficacy:

The Intent-To-Treat population (Full Analysis Set) was defined as all randomized subjects with a baseline iPTH and at least 2 on-treatment iPTH measurements. This population was used in the primary efficacy analysis.

The primary efficacy analysis was a comparison between the Paricalcitol and placebo treatment groups of the proportion of subjects achieving 2 consecutive decreases from baseline in iPTH of at least 30%.

This comparison was performed using a Fisher's exact test.

All randomized subjects who received at least 1 dose of study drug were used in secondary efficacy analyses.

Secondary efficacy analyses were performed comparing changes/percent change from baseline between the Paricalcitol and placebo treatment groups using a one-way ANOVA with treatment group as the factor for the following variables: iPTH and biochemical bone activity markers.

Safety:

All randomized subjects who received at least 1 dose of study drug were used in safety analyses.

The primary safety analysis was a comparison between the Paricalcitol and placebo treatment groups of the proportion of subjects achieving clinically meaningful hypercalcemia (2 consecutive calcium measurements >10.5 mg/dL). This comparison was performed using a Fisher's exact test.

Secondary safety analyses were performed comparing changes/percent changes from baseline between the Paricalcitol and placebo treatment groups using a one-way ANOVA with treatment group as the factor for the following variables: hematology, complete chemistry, and urinalysis variables; 24-hour urine collections, eGFR, urinary calcium/creatinine ratio, cardiovascular markers, and vital signs.

Secondary safety analyses also consisted of an analysis of “treatment-emergent” adverse events (i.e., adverse events with an onset date on or after the date the first dose of study drug was taken). Adverse events were summarized by body system and COSTART term according to the COSTART V adverse event-coding dictionary. Comparisons of the percentage of subjects experiencing an adverse event between the Paricalcitol and placebo treatment groups were performed using a Fisher's exact test.

Conclusions:

Efficacy Results:

A statistically significantly (p<0.001) greater proportion of subjects treated with Paricalcitol (initially dosed according to baseline iPTH values) had 2 consecutive ≧30% decreases from baseline in iPTH compared with subjects who received placebo (30/33, 91% versus 4/38, 11%). Additionally, in an exploratory analysis to evaluate the robustness of the primary efficacy analysis, a statistically significantly (p<0.001) greater proportion of Paricalcitol subjects had 4 consecutive ≧30% decreases from baseline in iPTH compared with placebo subjects (23/33, 70% versus 0/38, 0%). There was a statistically significant difference between the Paricalcitol and placebo treatment groups in mean change from baseline to Final Visit in iPTH using ANOVA with treatment as the factor.

Paricalcitol-treated subjects had a mean decrease (−46.9 pg/mL, representing a 15.2% decrease) in iPTH at the Final Visit compared with a mean increase (52.6 pg/mL, representing a 19.1% increase) among placebo-treated subjects. Similarly, Paricalcitol-treated subjects had a statistically significant mean decrease (−130.8 pg/mL, representing a 50.0% decrease) in iPTH at the Last On-Treatment Visit compared with a mean increase (61.1 pg/mL, representing a 21.4% increase) among placebo-treated subjects. The larger mean decrease and mean percent decrease using the Last On-Treatment Visit may be more representative of a treatment effect.

Statistically significant differences were observed between the Paricalcitol and placebo treatment groups at all scheduled visits of the Treatment Phase for both change and percent change from baseline in iPTH.

In paricalcitol-treated subjects, decreases in iPTH were observed as early as Week 3 (the first time iPTH was measured after the first dose) and continued throughout the

Treatment Phase. A 30% mean reduction in iPTH occurred by Week 7 and the maximum decrease (−52.4%) from baseline in iPTH was observed at Week 23.

Statistically significant differences were observed between the paricalcitol and placebo treatment groups in mean changes from baseline to Final Visit for the serum biochemical bone activity markers of serum osteocalcin and serum bone-specific alkaline phosphatase. Paricalcitol-treated subjects had mean decreases in serum osteocalcin and serum bone-specific alkaline phosphatase, while placebo subjects experienced a mean increase in serum osteocalcin and a small mean decrease in serum bone-specific alkaline phosphatase. Serum bone-specific alkaline phosphatase and osteocalcin are currently considered more sensitive and specific bone markers to evaluate the degree of bone remodeling in the setting of CKD than urine bone markers. The favorable result observed in the paricalcitol group suggests correction of high turnover bone disease associated with 2° HPT.

Safety Results:

No statistically significant differences were observed between the treatment groups for the overall incidence of adverse events or for the incidence of any specific adverse event. Treatment-emergent adverse events were experienced by 91% of paricalcitol subjects and 85% of placebo subjects. The majority of the adverse events reported in both treatment groups were mild or moderate in severity (93% paricalcitol and 95% placebo) and considered by the Investigator to be not related to study drug administration (67% paricalcitol and 59% placebo). The most commonly reported adverse events in the paricalcitol group were accidental injury (17%), pharyngitis (14%), diarrhea, edema, rash, vomiting (11% each), abdominal pain, allergic reaction, cough increased, and nausea (9% each). The most commonly reported adverse events in the placebo group were pharyngitis, pain (13% each), viral infection, uremia, myalgia (10% each), accidental injury, diarrhea, edema, vomiting, and gastritis (8% each). One (1) paricalcitol subject died due to hepatic encephalopathy that was considered not related to study drug. Overall, 15 subjects (7 paricalcitol and 8 placebo) reported serious adverse events, including the 1 death, during the Treatment and Follow-Up Phases of the study. None of the serious adverse events was considered by the Investigator to have a causal relationship to study drug. Six (6) subjects (4 paricalcitol and 2 placebo) were listed as having terminated prematurely from the study due to adverse events. The only event leading to premature termination considered by the Investigator to have a causal relationship (probably related) to study drug was allergic reaction, which was reported by 1 paricalcitol subject.

No subjects in either treatment group developed clinically meaningful hypercalcemia, defined as at least 2 consecutive calcium values >10.5 mg/dL (0/35 Paricalcitol subjects, 0/40 placebo subjects).

No statistically significant differences were observed between the paricalcitol and placebo treatment groups in mean change from baseline to Final Visit in calcium and Ca×P. Both treatment groups experienced mean decreases from baseline in calcium. The paricalcitol group experienced a mean decrease from baseline in Ca×P, while the placebo group experienced a mean increase from baseline in Ca×P. A statistically significant difference was observed between the paricalcitol and placebo treatment groups in mean change from baseline to Final Visit in phosphorus. A mean decrease in phosphorus (−0.13 mg/dL) was observed in the paricalcitol group compared to a mean increase in phosphorus (0.31 mg/dL) observed in the placebo group.

For serum calcium, mean change from baseline to Last On-Treatment Visit was statistically significantly different between the treatment groups; a small increase in calcium was observed in the paricalcitol group (0.21 mg/dL) and a small mean decrease in calcium was observed in the placebo group (−0.12 mg/dL), suggesting a minimal effect of treatment on serum calcium. The mean decrease in calcium observed in the placebo group is consistent with the pathogenesis of 2° HPT and reflects the disease state in this subject population. Although statistically significant, the minimal difference between the paricalcitol and placebo group in calcium is not considered clinically significant. Mean changes from baseline to Last On-Treatment Visit in phosphorus or Ca×P were not statistically significantly different between the 2 treatment groups. Statistically significant differences between the paricalcitol and placebo treatment groups were observed at Weeks 7, 11, 13, 15, 17, 19, 21, and 23 during the Treatment Phase for mean change from baseline in calcium values, with small mean increases in calcium values observed in the paricalcitol group and small mean decreases in calcium values observed in the placebo group. Mean calcium values ranged from 9.24 to 9.54 mg/dL in the paricalcitol group and 9.05 to 9.28 mg/dL in the placebo group. Other than the statistically significant differences noted between the paricalcitol and placebo groups at Weeks 13 and 15 of the Treatment Phase for Ca×P, no other statistically significant differences between the paricalcitol and placebo groups were observed during the Treatment Phase for phosphorus or Ca×P.

A statistically significant difference was observed between the treatment groups in mean change from baseline to Final Visit in alkaline phosphatase. A mean decrease from baseline in alkaline phosphatase was observed in the paricalcitol treatment group while a mean increase from baseline in this variable was observed in the placebo group. A decrease in alkaline phosphatase parallels the decrease in bone specific alkaline phosphatase supporting improvement in bone abnormalities associated with 2° HPT. No statistically significant differences were observed between the treatment groups in mean change or mean percent change from baseline to Final Visit in eGFR and serum creatinine for all subjects who completed 24 weeks of treatment. Additionally, no statistically significant difference was observed between the treatment groups in mean change from baseline to Final Visit in 24-hour urine collection variables (calcium, phosphorus, Ccr) or urinary calcium/creatinine ratio. Therefore, a paricalcitol treatment effect was not detected for urinary calcium and phosphorus excretion as well as kidney function parameters (eGFR, Ccr, serum creatinine).

Evaluations of other laboratory analyses, vital signs and physical examinations revealed no clinically meaningful changes as a result of paricalcitol treatment.

Conclusions:

Paricalcitol capsule is safe and well tolerated for the treatment and prevention of 2° HPT in CKD (Stages 3 and 4) subjects.

Paricalcitol capsule is effective for the treatment and prevention of 2° HPT in CKD (Stages 3 and 4) subjects.

When paricalcitol capsule was initially dosed according to severity of the 2° HPT, a statistically significantly (p<0.001) greater proportion of subjects had 2 consecutive ≧30% decreases from baseline in iPTH compared with subjects who received placebo (30/33, 91% versus 4/38, 11%). Statistically significant differences were observed between the paricalcitol and placebo treatment groups at all scheduled visits of the Treatment Phase for both change and percent change from baseline in iPTH. In Paricalcitol-treated subjects, decreases in iPTH were observed as early as Week 3 (the first time iPTH was measured after the first dose). Clinically meaningful suppression of iPTH (a 30% decrease from baseline in iPTH) was achieved within 7 weeks of treatment and was observed throughout the remainder of the Treatment Phase.

Serum alkaline phosphatase and biochemical bone activity markers (serum osteocalcin and serum bone-specific alkaline phosphatase), which are used commonly to monitor bone remodeling activity in patients with metabolic bone disease, were reduced significantly in paricalcitol-treated subjects compared to placebo-treated subjects. The favorable result observed in the paricalcitol group suggests correction of high-turnover bone disease associated with 2° HPT. No subjects in either treatment group developed clinically meaningful hypercalcemia (2 consecutive calcium values >10.5 mg/dL). No statistically significant differences were observed between the treatment groups in mean change and mean percent change from baseline to Final Visit in eGFR or serum creatinine. Additionally, no statistically significant differences were observed between the treatment groups in mean change from baseline to Final Visit in 24-hour urine collection variables (calcium, phosphorus, Ccr) or urinary calcium/creatinine ratio. Thus, no deterioration in kidney function parameters (eGFR, Ccr, and serum creatinine) was observed among subjects treated with Paricalcitol compared with subjects treated with placebo.

### EXAMPLE 9

### Safety and Bioavailability of Oral Formulations of Paricalcitol in Subjects with End-Stage CKD Undergoing Continuous Peritoneal Treatment

In this example, a study was conducted to assess the safety and bioavailability of a paricalcitol capsule formulation relative to that of a paricalcitol intravenous formulation in subjects with end-stage CKD undergoing continuous dialysis peritoneal (abbreviated as “CPD”) treatment.

Methodology: This was a Phase I, open-label, randomized, single-dose, two-period, crossover, nonfasting study. Subjects were randomized into two sequence groups of

Formulations A and B. The two nonfasting study administrations were:

Formulation A: Paricalcitol capsule formulation (0.24 μg/kg) administered orally with 180 mL of water (test). The strengths of the capsule formulations were 0.5, 1, 2 or 4 μg.

Formulation B: Paricalcitol intravenous formulation (0.24 μg/kg) administered as an intravenous bolus injection in a strength of 5 μg/mL (reference). The intravenous formulation contained 2-10 micrograms/milliliter of paricalcitol, 30% (v/v) propylene glycol, 20% (v/v) ethanol and 50% (v/v) water.

Both formulations were administered immediately after CPD exchange in the morning, 30 minutes after breakfast was served. Phosphate binders, commonly used in the management of end-stage renal disease, were withheld 8 hours prior to and 2 hours after the drug administration. A washout interval of at least 7 days separated the doses of the two study periods.

For the paricalcitol intravenous formulation, the blood samples were collected into evacuated EDTA containing collection tubes, from the arm contralateral to the injection arm, immediately prior to dosing (0 hour) and at 5 and 30 minutes and at 1, 2, 3, 4, 6, 8, 12, 24 and 48 hours post-dose. The blood samples for the paricalcitol capsule formulation were collected immediately prior to dosing (0 hour) and at 30 minutes and at 1, 1.5, 2, 3, 4, 6, 8, 12, 24 and 48 hours post-dose.

Plasma concentrations of paricalcitol were determined using a validated HPLC-tandem mass spectrometric assay method at Abbott Laboratories, Abbott Park, Ill. The lower limit of quantitation of paricalcitol was 0.02 ng/mL using 0.6 mL of plasma.

Diagnosis and Main Criteria for Inclusion: Subjects were male and female volunteers between 18 and 75 years of age, inclusive. Subjects had end-stage renal disease and had undergone CPD for at least 8 weeks prior to entry into the study. Female subjects of childbearing potential were neither pregnant nor breast-feeding and used reliable forms of birth control. A serum calcium (Ca) level was ≦10.5 mg/dL and a calcium-phosphorous product (Ca×P) level was ≦70.

Number of Subjects:

Planned: 8 to 12 Entered: 8 Completed: 8 Evaluated for Safety: 8

Evaluated for Pharmacokinetics: 8

Reference Therapy, Dose/Strength/Concentration and Mode of Administration:

The oral administration for Formulation A was accomplished with a combination of 0.5, 1, 2 or 4 μg capsule strengths. The intravenous administration for Formulation B was accomplished with a 5 μg/mL intravenous formulation.

Duration of Treatment: Single dose on Study Day 1. Two and one-half days of confinement in each of two periods.

Criteria for Evaluation:

Pharmacokinetic: The pharmacokinetic parameter values of paricalcitol were estimated using noncompartmental methods. These included: Cₘₐₓ, t_{1/2}, AUC₀₋ₜ, AUC_{0-∞}, CL, CL/F, Tₘₐₓ, Vd_{β} and Vd_{β}/F, as described in the last example.

Safety: Safety was evaluated based upon vital signs, physical examinations, laboratory tests, electrocardiograms (ECGs) and adverse events assessment throughout the study.

Statistical Methods: An analysis of variance (ANOVA) was performed for β and the logarithms of Cₘₐₓ, AUC₀₋ₜ and AUC_{0-∞}. Within the framework of the ANOVA for the logarithms of Cₘₐₓ and AUC₀₋ₜ and AUC_{0-∞}, a 95% confidence interval for the bioavailability of the capsule formulation relative to that of the intravenous formulation was obtained. The number and percentage of subjects reporting adverse events were tabulated by COSTART term and body system. Laboratory values outside the reference ranges were flagged and evaluated for clinical significance.

Summary/Conclusions:

Pharmacokinetic Results: Mean±SD pharmacokinetic parameters of paricalcitol are listed in the following Table 14.

TABLE 14

| | Formulation A | Formulation B |
|---|---|---|
| | Paricalcitol | Paricalcitol |
| | Capsule | Intravenous |
| Pharmacokinetic | Formulation | Formulation |
| Parameters | (N = 8) | (N = 8) |
| Cₘₐₓ (ng/mL) | 0.413 ± 0.064 | 1.832 ± 0.315 |
| Tₘₐₓ (h) | 6.0 ± 3.3 | ND |
| AUC₀₋ₜ (ng · h/mL) | 9.66 ± 2.51 | 13.01 ± 4.31 |
| AUC_{0-∞} (ng · h/mL) | 13.41 ± 5.48 | 16.01 ± 5.98 |
| β (1/h) | 0.039 ± 0.02 | 0.045 ± 0.026 |
| t_{1/2} (h)†$ | 17.7 ± 9.6 | 15.4 ± 10.5 |
| CL (L/h)†φ | 1.76 ± 0.77 | 1.54 ± 0.95 |
| Vd_{β} (L)†φ | 48.7 ± 15.6 | 34.9 ± 9.5 |

Formulation A: Paricalcitol capsule formulation, 0.24 μg/kg (test).
Formulation B: Paricalcitol intravenous formulation, 0.24 μg/kg, (reference).
$Harmonic mean ± pseudo-standard deviation; evaluations of t_{1/2} were based on statistical tests for β.
†Parameter was not tested statistically.
φCL for Formulation B and CL/F for Formulation A; Vd_{β} for Formulation B and Vd_{β}/F for Formulation A.
ND: Not determined.

The absolute bioavailability results of paricalcitol are listed in the following Table 15.

TABLE 15

| | | Central Value* | | Absolute Bioavailability | |
|---|---|---|---|---|---|
| Formulations | Pharmacokinetic | Oral | Intravenous | Point | 95% Confidence |
| Test vs. Reference | Parameter | A | B | Estimate⁺ | Interval |
| A vs. B | Cₘₐₓ | 0.411 | 1.799 | 0.228 | 0.185-0.282 |
| | AUC₀₋ₜ | 9.007 | 11.453 | 0.786 | 0.667-0.927 |
| | AUC_{0-∞} | 11.771 | 13.671 | 0.861 | 0.665-1.115 |

*Antilogarithm of the least squares means for logarithms.
⁺Antilogarithm of the difference (test minus reference) of the least squares means for logarithms.

Safety Results: For oral dosing of paricalcitol (Formulation A), six subjects (75%) reported at least one adverse event. For intravenous dosing of paricalcitol (Formulation B), seven subjects (87.5%) reported at least one adverse event. The most frequently reported adverse events for subjects receiving Formulation A were back pain, pain and ecchymosis (two subjects per adverse event; 25%) and no adverse events were considered possibly or probably related to the paricalcitol in this group. The most frequently reported adverse event in subjects receiving Formulation B was application site reaction, associated with intravenous administration of paricalcitol injection (seven subjects; 87.5%). Adverse events having probable or possible relationship to the paricalcitol for intravenous dosing were pain, hypercalcemia, neuralgia, application site reaction and taste perversion. One serious adverse event (peritonitis) occurred for intravenous dosing, but it was considered not related to the paricalcitol.

No deaths or premature discontinuations occurred during the study. One subject experienced mild elevations of calcium that were considered as an adverse event and possibly related to the paricalcitol. No other changes in laboratory measurements were clinically significant. No physical examination results, ECG changes, or changes in vital signs were clinically significant.

Conclusions: In subjects with end-stage renal disease who were undergoing CPD, the absolute bioavailability of paricalcitol administered as a single oral dose of capsule formulation was estimated to be 86.1%. The harmonic mean of the terminal elimination half-life of paricalcitol was approximately 15 hours following intravenous administration and approximately 18 hours following oral administration.

The formulations tested were generally safe and well tolerated by the subjects. No new or unexpected patterns of adverse event occurrences were identified with the administration of paricalcitol capsule or intravenous formulations. Except for application site reaction observed during the administration of the paricalcitol injection, no apparent differences were identified between the paricalcitol capsule and injection with respect to safety.

### EXAMPLE 10

### Safety of Oral Paricalcitol Formulations in Phase 3 Trials in Chronic Kidney Disease (CKD) Stages 3-4 Subjects with Secondary Hyperparathyroidism

As discussed previously, Vitamin D deficiency is common in early stage CKD and is a major factor in the development of 2° HPT. However, few nephrologists prescribe calcitriol due to concerns of its calcemic, phosphatemic and calciuric effects and potential adverse effect on kidney function. In this example, a study was conducted that evaluated the safety of paricalcitol capsule in CKD stage 3-4 subjects with 2° HPT in 3 double-blind, placebo-controlled multicenter studies. A total of 220 subjects with iPTH ≧150 pg/mL, serum Ca 8-10 mg/dL and P≦5.2 mg/dL were randomized 1:1 and treated with paricalcitol capsule (N=107) or placebo (N=113) three times per week or daily for 24 weeks. Initial doses were based on iPTH levels at baseline (iPTH <500 pg/mL: 2 mcg TIW or 1 mcg QD; iPTH ≧500 pg/mL: 4 mcg TIW or 2 mcg QD). Subsequent dose adjustments (TIW: 2 mcg, QD: 1 mcg) were based on biweekly Ca, P and iPTH results, dose increases occurred every 4 weeks.

There were no statistically significant differences between the treatment groups in mean change from baseline to Final Visit in 24-hr urinary calcium and phosphorus excretion. Both treatment groups experienced mean decreases from baseline in eGFR (based on Modification of Diet in Renal Disease (MDRD) formula referred to in Levey A., et al., “A more accurate method to estimate glomerular filtration rate from serum creatinine: A new prediction equation,” Ann. of Med., 130(6):467-70 (1999), herein incorporated by reference.

The difference between paricalcitol and placebo treatment groups in mean change or percent change from baseline in eGFR was not statistically significant for all subjects who completed 24 weeks of treatment. The results are shown below in Table 16.

TABLE 16

| | Paricalcitol | Placebo | p-value^{a} |
|---|---|---|---|
| Urine Calcium (mg/24 hours) | | | |
| Mean Baseline Value | 39.6 | 37.46 | — |
| Mean Final Value | 42.0 | 37.1 | NA |
| Mean Change from | 2.4 (3.04) | −0.38 (2.926) | 0.521 |
| Baseline (SE) | | | |
| Urine Phosphorous | | | |
| (mg/24 hours) | | | |
| Mean Baseline Value | 672.5 | 691.6 | — |
| Mean Final Value | 670.4 | 725.8 | NA |
| Mean Change from | −2.1 (39.72) | 34.2 (37.50) | 0.508 |
| Baseline (SE) | | | |
| eGFR (mL/min/1.73 m², | | | |
| MDRD formula) | | | |
| Mean Baseline Value | 23.9 | 23.4 | — |
| Mean Final Value | 21.4 | 21.9 | NA |
| Mean Change from Baseline | −2.5 (0.526) | −1.5 (0.494) | 0.187 |
| Mean Percent Change from | −10.4 (2.268) | −7.0 (2.130) | 0.269 |
| Baseline (SE) | | | |

^{a}One-way ANOVA with treatment as the factor.

There was no statistically significant difference between the treatment groups in the incidence of hypercalcemia defined as 2 consecutive Ca >10.5 mg/dL (p=0.237). Two (2/106, 2%) paricalcitol subjects and none of the placebo subjects experienced hypercalcemia. The difference between the treatment groups in the incidence of elevated P (>5.5 mg/dL) and elevated Ca×P (>55 mg²/dL²) was not statistically different Treatment-emergent adverse events reporting were similar between paricalcitol and placebo subjects (82% vs. 76%). Pharyngitis was the most commonly reported adverse event in both treatment groups (10% paricalcitol and 11% placebo).

In conclusion, paricalcitol capsule is safe and well tolerated for the treatment of 20 HPT in CKD stage 3-4 subjects. No deterioration in kidney function parameters were detected among subjects treated with paricalcitol compared with subjects treated with placebo. Paricalcitol treatment did not increase the risk of hypercalcemia, elevated P or Ca×P.

### EXAMPLE 11

### Use of Oral Formulations of Paricalcitol to Effectively Control Secondary Hyperparathyroidism in Patients with Stage 3-4 CKD

Secondary hyperparathyroidism develops early in the course of CKD and progresses over time. A major factor implicated in its development and progression is diminished calcitriol synthesis. Relative or absolute vitamin D deficiency is common in early stage CKD. Although calcitriol can suppress PTH levels in CKD pre-dialysis patients, its associated side effects of hypercalcemia, hyperphosphatemia and the potential risk of deterioration in kidney function limit its clinical use.

In this example, three double-blind, placebo-controlled, multicenter studies were conducted to evaluate the safety and efficacy of paricalcitol capsule in CKD stage 3-4 subjects. Major inclusion included eGFR (MDRD formula) 15-60 mL/min/1.73 m², iPTH ≧150 pg/mL. A total of 220 subjects across the 3 studies were randomized and received paricalcitol capsule or placebo three times weekly (TIW, N=145) or daily (QD, N=75) for 24 weeks. Mean baseline eGFR was 23.90 mL/min/1.73 m² in the paricalcitol group and 23.44 mL/min/1.73 m² in the placebo group. The initial dose was 2 mcg (TIW) or 1 mcg (QD) for baseline iPTH ≦500 pg/mL and 4 mcg (TIW) or 2 mcg (QD) for baseline iPTH >500 pg/mL. Doses were titrated based on serum Ca, P and iPTH results that were measured every 2 weeks, dose increases occurred every 4 weeks.

Overall 92/101 (91%) of paricalcitol-treated subjects had 2 consecutive 30% decreases in iPTH compared to 14/108 (13%) of placebo subjects (p 0.001). Paricalcitol group had a 30% mean iPTH reduction by Week 9 and the reduction was sustained throughout the treatment. Changes in serum Ca, P and Ca×P were minimal in both treatment groups. No deterioration in kidney function parameters was detected among paricalcitol-treated subjects compared with placebo subjects. The results are shown below in Table 17.

TABLE 17

| End Points | Paricalcitol | Placebo | p-value^{a} |
|---|---|---|---|
| | N = 101 | N = 108 | |
| 2 consecutive 30% iPTH | 92 (91%) | 14 (13%) | <0.001 |
| reduction from baseline | | | |
| | N = 106 | N = 111 | |
| Hypercalcemia | 2 (2%) | 0 (0%) | 0.237 |
| (2 consecutive Ca > 10.5 mg/dL) | | | |
| Hyperphosphatemia | 11 (10%) | 13 (12%) | 0.830 |
| (2 consecutive P > 5.5 mg/dL) | | | |
| Elevated Ca × P | 13 (12%) | 7 (6%) | 0.161 |
| (2 consecutive Ca × P > 55 mg²/dL²) | | | |

^{a}One-way ANOVA with treatment as the factor.

In conclusion, paricalcitol capsule provides effective and sustained iPTH reduction in CKD stage 3-4 subjects with no significant difference in the incidence of hypercalcemia, hyperphosphatemia and elevated Ca×P as compared to placebo. Paricalcitol therapy does not negatively affect kidney function in CKD stage 3-4.

### EXAMPLE 12

### Equal Effectiveness of Oral Formulations of Paricalcitol Dosed Daily or Three Times a Week in Reducing iPTH Levels in CKD Stage 3-4 Subjects

Vitamin D compounds (Vitamin D receptor activators [VDRA]) dosed every other day, three times a week is a standard method for the treatment of 2° HPT in CKD stage 5. This dosing method produces higher blood concentration and enhances PTH suppression, while minimizing the effect on calcium and phosphorus load. In CKD pre-dialysis patients, daily dosing offers a viable option for improved compliance. Currently, no data exist that directly compare the effect of QD and TIW dosing in CKD stage 3-4 patients.

In this example, paricalcitol capsules were evaluated in 3 double-blind, placebo-controlled multicenter studies in CKD stage 3-4 subjects with 2° HPT; 2 of these studies were conducted with TIW dosing and 1 was done with QD dosing. Subjects with eGFR (MDRD formula) 15-60 ml/min/1.73 m², iPTH ≧150 pg/mL, serum Ca 8.0-10.0 mg/dL and PO₄≦5.2 mg/dL were randomized 1:1 and treated with paricalcitol capsule or placebo for 24 weeks. A total of 145 subjects used TIW regimen (paricalcitol: 72; placebo: 73) and 75 subjects (paricalcitol: 35; placebo: 40) used QD regimen. After the initial doses, doses were titrated based on serum Ca, P and iPTH results that were measured every 2 weeks; dose increases occurred once every 4 weeks and decreases every 2 weeks. Dosing administrations are shown below in Table 18.

TABLE 18

| | TIW regimen | QD regimen |
|---|---|---|
| Initial Dose: | | |
| Baseline iPTH ≦ 500 pg/mL | 2 mcg | 1 mcg |
| Baseline iPTH > 500 pg/mL | 4 mcg | 2 mcg |
| Dose Adjustment: | 2 mcg | 1 mcg |

Overall, the proportion of paricalcitol-treated subjects who achieved 2 consecutive 30% decreases in iPTH was identical with TIW and QD regimen. (TIW: 62/68, 91%; QD: 30/33, 91%). The group receiving paricalcitol had a 30% mean iPTH reduction by Week 9 in TIW regimen and by Week 7 in QD regimen, and the reductions were sustained throughout the treatment. Changes in serum Ca, P and Ca×P were similar in both dosing regimen. The results are shown below in Table 19.

TABLE 19

| | TIW regimen (N = 72) | | QD regimen (N = QD) | | |
|---|---|---|---|---|---|
| End points | Paricalcitol | Placebo | Paricalcitol | Placebo | p-value^{a} |
| Mean average weekly dose | 9.5 ± (3.60) | 17.3 ± (5.32) | 9.6 ± (4.30) | 19 ± (5.97) | — |
| Median | 8.9 | | 9.3 | | |
| Range | 2.0-21.0 | | 3.1-22.3 | | |
| 2 consecutive 30% iPTH | 91% | 14% | 91% | 11% | TBP |
| reduction from baseline | | | | | |
| Hypercalcemia | 2 (3%) | 0 (0%) | 0 (%) | 0 (0%) | TBP |
| (2 consecutive Ca > 10.5 mg/dL) | | | | | |
| Hyperphosphatemia | 6 (9%) | 8 (11%) | 5 (14%) | 5 (13%) | 0.641 |
| (2 consecutive P > 5.5 mg/dL) | | | | | |
| Elevated Ca × P | 8 (12%) | 5 (7%) | 5 (14%) | 2 (5%) | 0.583 |
| (2 consecutive Ca × P > 55 mg²/dL²) | | | | | |
| Days in Treatment | | | | | |
| Mean (SD) | 148 (42.7) | | 146 (41.7) | | |
| Median | 166 | | 167 | | |

^{a}p-value derived from Breslow-Day Test.

TABLE 20
Changes from Baseline in Efficacy Variables by Treatment Regimen

| | TIW Regimen | | | QD Regimen | | |
|---|---|---|---|---|---|---|
| | | | ANOVA | | | ANOVA |
| | Paricalcitol | Placebo | p-value^{a} | Paricalcitol | Placebo | p-value^{a} |
| iPTH (pg/mL) | N = 69 | N = 71 | | N = 35 | N = 39 | |
| Mean Baseline Value | 269 | 294 | — | 259 | 253 | — |
| Mean Last On-Treatment | 179 | 315 | — | 128 | 315 | — |
| Value | | | | | | |
| Mean Change from Baseline | −90 (10.7) | 21 (10.6) | <0.001 | −131 (16.7) | 61 (15.8) | <0.001 |
| (SE) | | | | | | |
| Mean Percent Change from | −33% (3.6) | 7% (3.6) | <0.001 | −50% (5.0) | 21% (4.7) | <0.001 |
| Baseline (SE) | | | | | | |
| Bone Spec. Alk Phos (mcg/L) | N = 68 | N = 70 | | N = 33 | N = 37 | |
| Mean Baseline Value | 16.7 | 19.8 | — | 17.9 | 17.1 | — |
| Mean Change from Baseline | −8.0 (0.90) | −2.0 (0.89) | <0.001 | −7.6 (1.40) | −0.3 (1.32) | <0.001 |
| Serum Osteocalcin (ng/mL) | N = 67 | N = 67 | | N = 33 | N = 37 | |
| Mean Baseline Value | 58.0 | 68.1 | — | 72.3 | 76.1 | — |
| Mean Change from Baseline | −19.0 (2.94) | 12.9 (2.94) | <0.001 | −27.1 (5.54) | 6.9 (5.24) | <0.001 |

^{a}One-way ANOVA with treatment as the factor.

Safety Results

Regardless of the treatment regimen, there were no statistically significant differences in the rate of clinically meaningful hypercalcemia, elevated phosphorus or elevated Ca×P in paricalcitol patients compared to placebo. No differences in the effect of treatment regimen were detected. Results are presented in Table 21.

TABLE 21
Elevations of Calcium, Phosphorus and Ca×P

| | TIW Regimen | | | QD regimen | | | |
|---|---|---|---|---|---|---|---|
| | Paricalcitol | Placebo | | Paricalcitol | Placebo | | Homogeneity |
| End points | (N = 69) | (N = 71) | p-value | (N = 35) | (N = 39) | p-value | p-value^{a} |
| 2 consecutive Ca > | 2 (3%) | 0 (0%) | 0.241 | 0 (%) | 0 (0%) | NA | Not |
| 10.5 mg/dL | | | | | | | performed^{b} |
| 2 consecutive P > | 6 (9%) | 8 (11%) | 0.780 | 5 (14%) | 5 (13%) | 1.000 | 0.641 |
| 5.5 mg/dL | | | | | | | |
| 2 consecutive CaxP > | 8 (12%) | 5 (7%) | 0.396 | 5 (14%) | 2 (5%) | 0.245 | 0.583 |
| 55 mg²/dL² | | | | | | | |

^{a}Test of homogeneity of odds ratios from Brelow-Day Test
^{b}Breslow-Day Test was not computed on calcium because the data were too sparse to calculate.

In conclusion, paricalcitol capsule, dosed with QD or TIW regimen is equally safe and effective for the treatment of 2° HPT in subjects with CKD stage 3-4.

### EXAMPLE 13

### Comparison of Intravenous and Oral Formulations of Various Vitamin D Receptor Activators and Selective Vitamin D Receptor Activators

Table 22 provides a summary of the incidences of hypercalcemia and hypercalcemia resulting from intravenous and oral administration of Zemplar®, intravenous and oral administration of Hectorol®, intravenous and oral administration of One-Alpha® and intravenous and oral administration of calcitriol (Calcijex® IV and Rocaltrol® oral). This information demonstrates that similar molecules exhibit different clinical effects if administered by different formulations and that oral and intravenous formulations of paricalcitol exhibit similar clinical effects. This information was compiled from the following documents: Rocaltrol® Summary Basis of Approval (SBA), Supplemental NDA, Calcitriol® (Oral) NDA 18-0444, Submission Nov. 18, 1997; Hectorol SBA, Doxercalciferol Injection, NDA 21-027, Submission Dec. 20, 1999; Maung HM, Elangovan L, Frazao JM, “Efficacy and side effects of intermittent intravenous and oral doxercalciferol in dialysis patients with secondary hyperparathyroidism: A sequential comparison”, Am. J. Kidney Dis., 37:532-543 (2001); Urena P, Bernard-Poenaru O, Cohen-Solal M., “Plasma bone-specific alkaline phosphatase changes in hemodialysis patients treated by alfacalcidol,” Clin Nephrol., 57:261-273, 2002; Rapport J, Mostoslavski M, Ben-David A., “Successful treatment of secondary hyperparathyroidism in hemodialysis patients with oral pulse 1-alpha-hydrox-cholecalciferol therapy,” Nephrol. Dial. Transplant, 17 (Suppl 10): 28-36, 2002; Sprague SM, Llach F, Amdahl M., “Paricalcitol versus calcitriol in the treatment of secondary hyperparathyroidism,” Kidney Int., 63: 1483-1490, 2003.

TABLE 22

| | | | | % of Patients | Incidence of | Hyper- | Incidence | Hyperphospha- |
|---|---|---|---|---|---|---|---|---|
| | | | Efficacy | Achieving | Hyper- | calcemia | of | temia |
| | Mean iPTH | | Endpoint | Efficacy | calcemia | Defined As: | hyperphosphatemia | Defined A |
| | (pg/mL) | | iPTH | Endpoint | (mg/dL) | (mg/dL) | (mg/dL) | (mg/dL) |
| Zemplar ® IV | Baseline | 783 | At least one | 88 | 5% | Ca > 11.5 | N/A | N/A |
| | Final | 404 | decrease ≧30% | | | | | |
| | Change | −379 | | | | | | |
| Zemplar ® Oral | Baseline | 617 | Two consecutive | 91 | 6% | Two | Not Known | Two |
| | Final | 366 | decreases ≧30% | | | consecutive | | consecutive |
| | Change | −251 | | | | Ca > 11.2 | | P > 6.9 |
| Hectorol ® IV | Baseline | 748 ± 49.5 | At least one | 78 | 34.3% | Ca > 10.5 | 54.3% | P > 6.9 |
| | Final | 429.4 ± 27.4 | decrease ≧50% | | | | | |
| | Change | −318.6 | | | | | | |
| Hectorol ® Oral | Baseline | 950 ± 67.2 | At least one | 89 | 44.3% | Ca > 10.5 | 65.7% | P > 6.9 |
| | Final | 407.6 ± 25.3 | decrease ≧50% | | | | | |
| | Change | −542.4 | | | | | | |
| One-Alpha ® IV | Baseline | 826 ± 300 | 150-300 pg/mL | 20 | 20% | Ca > 11.0 | 50% | P > 6.2 |
| | Final | 436 ± 371 | | | | | | |
| | Change | −390 | | | | | | |
| One-Alpha ® Oral | Baseline | 515 ± 50 | <100 pg/mL | 58 | 55% | Ca > 11.0 | N.R. | P > 6.0 |
| | Final | 164 ± 39 | | | | | | |
| | Change | −351 | | | | | | |
| Calcijex ® IV | Baseline | 675 ± 35.0 | At least one | 62 | 10% | Ca > 11.5 | 50.8% (1P) 38% (↑ | Ca × P ≧ 75 |
| | Final | | decrease ≧50% | | | | Ca × P) | |
| | Change | | | | | | | |
| Rocaltrol ® Oral | Baseline | N/A | Any reduction | Not | 64% | Ca > 10.8 | N/A | N/A |
| | Final | | | significant | | | | |
| | Change | | | | | | | |

Capsules in CKD Stage 3 and Stage 4 Subjects Following Every-Day Dosing

Paricalcitol capsules are under development for the prevention and treatment of secondary hyperparathyroidism in chronic kidney disease (CKD). The aim of this open-label, single and multiple dose, multi-center study was to evaluate the safety, and pharmacokinetics (PK) of paricalcitol in CKD Stage 3 (single 4 μg dose on Day 1 and 4 μg QD from Days 3-8) and CKD Stage 4 (single 3 μg dose on Day 1 and 3 μg QD from Days 3-8) subjects. Plasma samples for paricalcitol levels were measured for 48 hrs after day 1 and day 8 doses using an LC-MS/MS assay with a lower limit of quantification of 0.01 ng/mL. The PK parameters of paricalcitol are listed in the following table.

TABLE 23

| PK | CKD Stage 3 (n = 15) | | CKD Stage 4 (n = 13) | |
|---|---|---|---|---|
| Parameter | Day 1 (3 μg) | Day 8 (3 μg QD) | Day 1 (4 μg) | Day 8 (4 μg QD) |
| Cₘₐₓ (ng/mL) | 0.113 ± 0.036 | 0.155 ± 0.057 | 0.065 ± 0.012 | 0.097 ± 0.023 |
| AUC_{0-inf}(ng · h/mL) | 2.424 ± 0.614 | ND | 2.127 ± 0.733 | ND |
| AUC₀₋₂₄ (ng · h/mL) | ND | 2.220 ± 0.701* | ND | 1.754 ± 0.421* |
| t_{1/2} (h) | 16.76 ± 2.65 | 15.53 ± 3.21 | 19.70 ± 7.19 | 22.95 ± 5.63 |
| CL/F (L/h) | 1.766 ± 0.505 | 2.014 ± 0.774 | 1.517 ± 0.359 | 1.751 ± 0.388 |

ND: Not determined;
*P < 0.05 from Day 1 AUC_{0-inf}

In CKD Stage3 and 4 subjects, paricalcitol steady state was essentially reached by Day 6. The mean paricalcitol exposure at steady state (AUC₀₋₂₄) was slightly lower than that of Day 1 AUC_{0-inf}, but the PK of paricalcitol was essentially time linear. The mean t_{1/2} of paricalcitol was approximately 16-23 h, similar to that of CKD Stage 5 subjects. No safety concerns were observed after repeated dosing of paricalcitol in CKD Stage 3 and 4 subjects.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

What is claimed is:

1. Any member of a family of oral formulations comprising:
a amount of paricalcitol dissolved in an amount of a non-polar solvent, wherein each family member comprises a ratio of non-polar solvent to paricalcitol and said ratio does not vary by more than a factor of up to about 4 to a ratio of non-polar solvent to paricalcitol in a selected oral reference formulation that is a member of the family and each family member, when dosed at the same total weight of paricalcitol, is bioequivalent to the selected reference formulation and to one another.

2. Any member of a family of oral dosage forms according to claim 1, wherein AUC_{0-∞} of the family member is within 80% to 125% of AUC_{0-∞} of another family member.

3. Any member of a family of oral dosage forms according to claim 1, wherein AUC₀₋ₜ of the family member is within 80% to 125% of AUC₀₋ₜ of another family member.

4. Any member of a family of oral dosage forms according to claim 1, wherein Cₘₐₓ is within 80% to 125% of Cₘₐₓ of another family member.

5. Any member of a family of oral dosage forms according to claim 1, wherein said ratio does not vary by more than a factor of about 3.5 to a ratio of a non-polar solvent to paricalcitol.

6. Any member of a family of oral dosage forms according to claim 1, wherein said ratio does not vary by more than a factor of about 3 to a ratio of a non-polar solvent to paricalcitol.

7. Any member of a family of oral dosage forms according to claim 1, wherein said ratio does not vary by more than a factor of about 2.5 to a ratio of a non-polar solvent to paricalcitol.

8. Any member of a family of oral formulations comprising:
a) about 0.25 mcg of paricalcitol dissolved in an amount of a non-polar solvent;
b) about 0.50 mcg of paricalcitol dissolved in an amount of a non-polar solvent;
c) about 0.75 mcg of paricalcitol dissolved in an amount of a non-polar solvent;
d) about 1.0 mcg of paricalcitol dissolved in an amount of a non-polar solvent;
e) about 2.0 mcg of paricalcitol dissolved in an amount of a non-polar solvent;
f) about 3.0 mcg of paricalcitol dissolved in an amount of a non-polar solvent;
g) about 4.0 mcg of paricalcitol dissolved in an amount of a non-polar solvent;
h) about 8.0 mcg of paricalcitol dissolved in an amount of a non-polar solvent;
i) about 16.0 mcg of paricalcitol dissolved in an amount of a non-polar solvent; or
j) about 32.0 mcg of paricalcitol dissolved an amount of a non-polar solvent,
wherein each family member comprises a ratio of non-polar solvent to paricalcitol and said ratio does not vary by more than a factor of about 4 to a ratio of non-polar solvent to paricalcitol in a selected oral reference formulation that is a member of the family and each family member, when dosed at the same total weight of paricalcitol, is bioequivalent to the selected reference formulation and to one another.

9. A family of oral formulations made by a method comprising the steps of:
a) providing a first oral formulation comprising paricalcitol and a non-polar solvent, wherein said first oral formulation contains a first ratio of non-polar solvent to paricalcitol;
b) preparing any number of additional oral formulations comprising paricalcitol and a non-polar solvent, wherein each said additional oral formulation comprises a second ratio of non-polar solvent to paricalcitol and further wherein the second ratio does not differ by more than a factor of about 4 to the first ratio;
wherein each of said first and additional oral formulations of said family prepared pursuant to steps a) and b), when dosed at the same total weight of paricalcitol, are bioequivalent to each other.

10. A method of making a family of oral formulations that are bioequivalent, the method comprising the steps of:
a) providing a first oral formulation comprising paricalcitol and a non-polar solvent, wherein said first oral formulation contains a first ratio of non-polar solvent to paricalcitol;
b) preparing any number of additional oral formulations comprising paricalcitol and a non-polar solvent, wherein each said additional oral formulation comprises a second ratio of non-polar solvent to paricalcitol and further wherein the second ratio does not differ by more than a factor of about 4 to the first ratio;
wherein each of said first and additional oral formulations prepared pursuant to steps a) and b) are bioequivalent to each other.

11. A method of making a family of oral formulations that are bioequivalent, the method comprising the steps of:
a) providing a first oral formulation comprising paricalcitol and a non-polar solvent, wherein said first oral formulation contains a first ratio of non-polar solvent to paricalcitol;
b) preparing a second oral formulation comprising paricalcitol and a non-polar solvent, wherein said second oral formulation comprises a second ratio of non-polar solvent to paricalcitol and further wherein the first ratio does not differ by more than a factor of about 4 to the second ratio; and
c) preparing a third oral formulation comprising paricalcitol and a non-polar solvent, wherein said third oral formulation comprises a third ratio of non-polar solvent to paricalcitol and further wherein the third ratio does not differ by more than a factor of about 4 to the first ratio,
wherein each of said first, second and third formulations prepared pursuant to steps a), b) and c), when dosed at the same total weight of paricalcitol, are bioequivalent to each other.

12. A method of suppressing parathyroid hormone in patients suffering from chronic kidney disease and in need of treatment, the method comprising the step of orally administering any member of the family of oral formulations of claim 1 to said patient.

13. A method of reducing hospitalizations in patients suffering from chronic kidney disease and in need of treatment, the method comprising the step of orally administering any member of the family of oral formulations of claim 1 to said patient.

14. A method of preventing progression of kidney disease in patients suffering from chronic kidney disease and in need of treatment, the method comprising the step of orally administering any member of the family of oral formulations of claim 1 to said patient.

15. A method of reducing deaths in patients suffering from chronic kidney disease and in need of treatment, the method comprising the step of orally administering any member of the family of oral formulations of claim 1 to said patient.

16. A method of preventing cardiovascular disease in patients in need of treatment, the method comprising the step of orally administering any member of the family of oral formulations of claim 1 to said patient.

17. The method of claims 6-10 wherein the patient is a mammal.

18. The method of claims 6-9 wherein the chronic kidney disease is pre-end stage or end-stage renal disease.

19. The method of claims 6-10 wherein any member of the family of oral formulations of claim 1 is administered daily or three times a week.
